SEQUENCE LISTING (86 sequences)

SEQ ID NO: 1 - RNA, 35 nt, synthetic construct
  source 1..35: mol_type = other RNA
tgagagagacggattccgtccgcgaattcacgctg

SEQ ID NO: 2 - RNA, 35 nt, synthetic construct
  source 1..35: mol_type = other RNA
tgtgagagacggattccgtccgcgaattcacgctg

SEQ ID NO: 3 - RNA, 35 nt, synthetic construct
  source 1..35: mol_type = other RNA
tgagtgagacggattccgtccgcgaattcacgctg

SEQ ID NO: 4 - RNA, 35 nt, synthetic construct
  source 1..35: mol_type = other RNA
tgagacagacggattccgtccgcgaattcacgctg

SEQ ID NO: 5 - RNA, 35 nt, synthetic construct
  source 1..35: mol_type = other RNA
tgagagtgacggattccgtccgcgaattcacgctg

SEQ ID NO: 6 - RNA, 35 nt, synthetic construct
  source 1..35: mol_type = other RNA
tgagagagacggattccgtcagcgaattcacgctg

SEQ ID NO: 7 - RNA, 35 nt, synthetic construct
  source 1..35: mol_type = other RNA
ggagagagacggattccgtccgcgaattcacgctt

SEQ ID NO: 8 - RNA, 35 nt, synthetic construct
  source 1..35: mol_type = other RNA
tgagagacacggattccgtgcgcgaattcacgctg

SEQ ID NO: 9 - RNA, 36 nt, synthetic construct
  source 1..36: mol_type = other RNA
tgagagagacggttcgccgtccgcgaattcacgctg

SEQ ID NO: 10 - RNA, 35 nt, synthetic construct
  source 1..35: mol_type = other RNA
tgagagagacggattccgtccgcgtttcgacgctg

SEQ ID NO: 11 - RNA, 35 nt, synthetic construct
  source 1..35: mol_type = other RNA
tgagagagacggattccgtccgcgaattcgcgctg

SEQ ID NO: 12 - RNA, 35 nt, synthetic construct
  source 1..35: mol_type = other RNA
tgagagagacggattccgtccgcgtattcacgctg

SEQ ID NO: 13 - RNA, 49 nt, synthetic construct
  source 1..49: mol_type = other RNA
gtaatgttgagagagacggattccgtccgcgaattcacgctgacattac

SEQ ID NO: 14 - RNA, 51 nt, synthetic construct
  source 1..51: mol_type = other RNA
gtaatgtgtgagagagacggattccgtccgcgaattcacgctgcacattac

SEQ ID NO: 15 - RNA, 53 nt, synthetic construct
  source 1..53: mol_type = other RNA
gtaatgtggtgagagagacggattccgtccgcgaattcacgctgccacattac

SEQ ID NO: 16 - RNA, 55 nt, synthetic construct
  source 1..55: mol_type = other RNA
gtaatgtggctgagagagacggattccgtccgcgaattcacgctggccacattac

SEQ ID NO: 17 - RNA, 57 nt, synthetic construct
  source 1..57: mol_type = other RNA
gtaatgtggcatgagagagacggattccgtccgcgaattcacgctgtgccacattac

SEQ ID NO: 18 - RNA, 54 nt, synthetic construct
  source 1..54: mol_type = other RNA
gtaatgtggtgagagagacggattccgtccgcgaattcacgctgccgcattgcc

SEQ ID NO: 19 - RNA, 118 nt, synthetic construct
  source 1..118: mol_type = other RNA
tctagaccctgcgtcacaacgacgtgagagagacggattccgtccgcgaattcacgctgcgtcgtctgggcgacggtaaataggtgttaggcccagagcggcagggtacaactccgga

SEQ ID NO: 20 - RNA, 119 nt, synthetic construct
  source 1..119: mol_type = other RNA
tctagaccctgcgtcacagacgacgtgagagagacggattccgtccgcgaattcacgctgcgtcgtctgggcgacggtaaataggtgttaggcccagagcggcagggtacaactccgga

SEQ ID NO: 21 - RNA, 120 nt, synthetic construct
  source 1..120: mol_type = other RNA
tctagaccctgcgtcacaagacgacgtgagagagacggattccgtccgcgaattcacgctgcgtcgtctgggcgacggtaaataggtgttaggcccagagcggcagggtacaactccgga

SEQ ID NO: 22 - RNA, 121 nt, synthetic construct
  source 1..121: mol_type = other RNA
tctagaccctgcgtcacacagacgacgtgagagagacggattccgtccgcgaattcacgctgcgtcgtctgggcgacggtaaataggtgttaggcccagagcggcagggtacaactccgga

SEQ ID NO: 23 - RNA, 117 nt, synthetic construct
  source 1..117: mol_type = other RNA
tctagaccctgcgtcacaccagacgacgtgagagagacggattccgtccgcgaattcacgctgcgtcgtctgggcgacggtaaataggtgttaggcccagagcggcagggtacaact

SEQ ID NO: 24 - RNA, 123 nt, synthetic construct
  source 1..123: mol_type = other RNA
tctagaccctgcgtcacacccagacgacgtgagagagacggattccgtccgcgaattcacgctgcgtcgtctgggcgacggtaaataggtgttaggcccagagcggcagggtacaactccgga

SEQ ID NO: 25 - RNA, 126 nt, synthetic construct
  source 1..126: mol_type = other RNA
tctagaccctgcgtcacaaagaaaagacgacgtgagagagacggattccgtccgcgaattcacgctgcgtcgtctgggcgacggtaaataggtgttaggcccagagcggcagggtacaactccgga

SEQ ID NO: 26 - RNA, 121 nt, synthetic construct
  source 1..121: mol_type = other RNA
tctagaccctgcgtcacatgagagagacggattccgtccgcgaattcacgctgtgtgacgcacgtcgtctgggcgacggtaaataggtgttaggcccagagcggcaaggtctaactccgga

SEQ ID NO: 27 - RNA, 121 nt, synthetic construct
  source 1..121: mol_type = other RNA
tctagaccctgcgtcacatgagagagacggattccgtccgcgaattcacgctgtgtgacgcacgtcgtctgggcgacggtaaataggtgttaggcccagagcggcagagtctaactccgga

SEQ ID NO: 28 - DNA, 44 bp, synthetic construct
  source 1..44: mol_type = other DNA
ttcaggtaatacgactcactatagggaagagaaggacatatgat

SEQ ID NO: 29 - DNA, 86 bp, synthetic construct
  source 1..86: mol_type = other DNA
tcaagtggtcatgtactagtcaannnnnnnnnnnnnnnnnnnnnnnnnnnnnnnnnnnnnnnnatcatatgtccttctcttcccta

SEQ ID NO: 30 - DNA, 23 bp, synthetic construct
  source 1..23: mol_type = other DNA
tcaagtggtcatgtactagtcaa

SEQ ID NO: 31 - RNA, 84 nt, synthetic construct
  source 1..84: mol_type = other RNA
gggaagagaaggacatatgatcaagtgagagagacggattccgtccgcgaattcacgctgcttgactagtacatgaccacttga

SEQ ID NO: 32 - DNA, 24 bp, synthetic construct
  source 1..24: mol_type = other DNA
tttttttttttttttttttttttt

SEQ ID NO: 33 - RNA, 69 nt, synthetic construct
  source 1..69: mol_type = other RNA
gcaagtgagagagacggattccgtccgcgaattcacgctgcttgcaaaaaaaaaaaaaaaaaaaaaaaa

SEQ ID NO: 34 - RNA, 69 nt, synthetic construct
  source 1..69: mol_type = other RNA
gcaagtcagagagacggattccgtccgcgaattcacgctgcttgcaaaaaaaaaaaaaaaaaaaaaaaa

SEQ ID NO: 35 - RNA, 69 nt, synthetic construct
  source 1..69: mol_type = other RNA
gcaagtgtgagagacggattccgtccgcgaattcacgctgcttgcaaaaaaaaaaaaaaaaaaaaaaaa

SEQ ID NO: 36 - RNA, 69 nt, synthetic construct
  source 1..69: mol_type = other RNA
gcaagtgacagagacggattccgtccgcgaattcacgctgcttgcaaaaaaaaaaaaaaaaaaaaaaaa

SEQ ID NO: 37 - RNA, 69 nt, synthetic construct
  source 1..69: mol_type = other RNA
gcaagtgagtgagacggattccgtccgcgaattcacgctgcttgcaaaaaaaaaaaaaaaaaaaaaaaa

SEQ ID NO: 38 - RNA, 69 nt, synthetic construct
  source 1..69: mol_type = other RNA
gcaagtgagacagacggattccgtccgcgaattcacgctgcttgcaaaaaaaaaaaaaaaaaaaaaaaa

SEQ ID NO: 39 - RNA, 69 nt, synthetic construct
  source 1..69: mol_type = other RNA
gcaagtgagagtgacggattccgtccgcgaattcacgctgcttgcaaaaaaaaaaaaaaaaaaaaaaaa

SEQ ID NO: 40 - RNA, 69 nt, synthetic construct
  source 1..69: mol_type = other RNA
gcaagtgagagagacggattccgtcagcgaattcacgctgcttgcaaaaaaaaaaaaaaaaaaaaaaaa

SEQ ID NO: 41 - RNA, 69 nt, synthetic construct
  source 1..69: mol_type = other RNA
gcaagtgagagagacggattccgtccgcgaattcacgcggcttgcaaaaaaaaaaaaaaaaaaaaaaaa

SEQ ID NO: 42 - RNA, 69 nt, synthetic construct
  source 1..69: mol_type = other RNA
gcaagggagagagacggattccgtccgcgaattcacgcttcttgcaaaaaaaaaaaaaaaaaaaaaaaa

SEQ ID NO: 43 - RNA, 69 nt, synthetic construct
  source 1..69: mol_type = other RNA
gcaagtgagagacacggattccgtgcgcgaattcacgctgcttgcaaaaaaaaaaaaaaaaaaaaaaaa

SEQ ID NO: 44 - RNA, 69 nt, synthetic construct
  source 1..69: mol_type = other RNA
gcaagtgagagagacggattccgtccccgaattcacggtgcttgcaaaaaaaaaaaaaaaaaaaaaaaa

SEQ ID NO: 45 - RNA, 70 nt, synthetic construct
  source 1..70: mol_type = other RNA
gcaagtgagagagacggttcgccgtccgcgaattcacgctgcttgcaaaaaaaaaaaaaaaaaaaaaaaa

SEQ ID NO: 46 - RNA, 69 nt, synthetic construct
  source 1..69: mol_type = other RNA
gcaagtgagagagacggattccgtccgcgtttcgacgctgcttgcaaaaaaaaaaaaaaaaaaaaaaaa

SEQ ID NO: 47 - RNA, 69 nt, synthetic construct
  source 1..69: mol_type = other RNA
gcaagtgagagagacggattccgtccgcgaattcacgccgcttgcaaaaaaaaaaaaaaaaaaaaaaaa

SEQ ID NO: 48 - RNA, 69 nt, synthetic construct
  source 1..69: mol_type = other RNA
gcaagtgagagagacggattccgtccgcgaattctcgctgcttgcaaaaaaaaaaaaaaaaaaaaaaaa

SEQ ID NO: 49 - RNA, 69 nt, synthetic construct
  source 1..69: mol_type = other RNA
gcaagtgagagagacggattccgtccgcgtattcacgctgcttgcaaaaaaaaaaaaaaaaaaaaaaaa

SEQ ID NO: 50 - RNA, 45 nt, synthetic construct
  source 1..45: mol_type = other RNA
gcaagtgagagagacggattccgtccgcgaattcacgctgcttgc

SEQ ID NO: 51 - DNA, 118 bp, synthetic construct
  source 1..118: mol_type = other DNA
tctagaccctgcgtcacaacgacgtgagagagacggattccgtccgcgaattcacgctgcgtcgtctgggcgacggtaaataggtgttaggcccagagcggcagggtacaactccgga

SEQ ID NO: 52 - DNA, 119 bp, synthetic construct
  source 1..119: mol_type = other DNA
tctagaccctgcgtcacagacgacgtgagagagacggattccgtccgcgaattcacgctgcgtcgtctgggcgacggtaaataggtgttaggcccagagcggcagggtacaactccgga

SEQ ID NO: 53 - DNA, 120 bp, synthetic construct
  source 1..120: mol_type = other DNA
tctagaccctgcgtcacaagacgacgtgagagagacggattccgtccgcgaattcacgctgcgtcgtctgggcgacggtaaataggtgttaggcccagagcggcagggtacaactccgga

SEQ ID NO: 54 - DNA, 121 bp, synthetic construct
  source 1..121: mol_type = other DNA
tctagaccctgcgtcacacagacgacgtgagagagacggattccgtccgcgaattcacgctgcgtcgtctgggcgacggtaaataggtgttaggcccagagcggcagggtacaactccgga

SEQ ID NO: 55 - DNA, 117 bp, synthetic construct
  source 1..117: mol_type = other DNA
tctagaccctgcgtcacaccagacgacgtgagagagacggattccgtccgcgaattcacgctgcgtcgtctgggcgacggtaaataggtgttaggcccagagcggcagggtacaact

SEQ ID NO: 56 - DNA, 123 bp, synthetic construct
  source 1..123: mol_type = other DNA
tctagaccctgcgtcacacccagacgacgtgagagagacggattccgtccgcgaattcacgctgcgtcgtctgggcgacggtaaataggtgttaggcccagagcggcagggtacaactccgga

SEQ ID NO: 57 - DNA, 126 bp, synthetic construct
  source 1..126: mol_type = other DNA
tctagaccctgcgtcacaaagaaaagacgacgtgagagagacggattccgtccgcgaattcacgctgcgtcgtctgggcgacggtaaataggtgttaggcccagagcggcagggtacaactccgga

SEQ ID NO: 58 - DNA, 121 bp, synthetic construct
  source 1..121: mol_type = other DNA
tctagaccctgcgtcacatgagagagacggattccgtccgcgaattcacgctgtgtgacgcacgtcgtctgggcgacggtaaataggtgttaggcccagagcggcaaggtctaactccgga

SEQ ID NO: 59 - DNA, 121 bp, synthetic construct
  source 1..121: mol_type = other DNA
tctagaccctgcgtcacatgagagagacggattccgtccgcgaattcacgctgtgtgacgcacgtcgtctgggcgacggtaaataggtgttaggcccagagcggcagagtctaactccgga

SEQ ID NO: 60 - DNA, 49 bp, synthetic construct
  source 1..49: mol_type = other DNA
gtaatgttgagagagacggattccgtccgcgaattcacgctgacattac

SEQ ID NO: 61 - DNA, 51 bp, synthetic construct
  source 1..51: mol_type = other DNA
gtaatgtgtgagagagacggattccgtccgcgaattcacgctgcacattac

SEQ ID NO: 62 - DNA, 53 bp, synthetic construct
  source 1..53: mol_type = other DNA
gtaatgtggtgagagagacggattccgtccgcgaattcacgctgccacattac

SEQ ID NO: 63 - DNA, 55 bp, synthetic construct
  source 1..55: mol_type = other DNA
gtaatgtggctgagagagacggattccgtccgcgaattcacgctggccacattac

SEQ ID NO: 64 - DNA, 57 bp, synthetic construct
  source 1..57: mol_type = other DNA
gtaatgtggcatgagagagacggattccgtccgcgaattcacgctgtgccacattac

SEQ ID NO: 65 - DNA, 54 bp, synthetic construct
  source 1..54: mol_type = other DNA
gtaatgtggtgagagagacggattccgtccgcgaattcacgctgccgcattgcc

SEQ ID NO: 66 - RNA, 64 nt, synthetic construct
  source 1..64: mol_type = other RNA
cgtcgtctgggcgacggtaaataggtgttaggcccagagcggcagggttcttccctgcgtcaca

SEQ ID NO: 67 - RNA, 114 nt, synthetic construct
  source 1..114: mol_type = other RNA
  misc_feature 1: note = EGFP sequence is linked to the 5' end.
  misc_feature 114: note = A poly-A tail is linked to the 3' end.
ctctagaccctgcgtcacagcagcatgagagagacggattccgtccgcgaattcacgctgcgtcgtctgggcgacggtaaataggtgttaggcccagagcggcagggtacaact

SEQ ID NO: 68 - RNA, 115 nt, synthetic construct
  source 1..115: mol_type = other RNA
  misc_feature 1: note = EGFP sequence is linked to the 5' end.
  misc_feature 115: note = A poly-A tail is linked to the 3' end.
ctctagaccctgcgtcacagcagcagtgagagagacggattccgtccgcgaattcacgctgcgtcgtctgggcgacggtaaataggtgttaggcccagagcggcagggtacaact

SEQ ID NO: 69 - RNA, 116 nt, synthetic construct
  source 1..116: mol_type = other RNA
  misc_feature 1: note = EGFP sequence is linked to the 5' end.
  misc_feature 116: note = A poly-A tail is linked to the 3' end.
ctctagaccctgcgtcacagcagcagatgagagagacggattccgtccgcgaattcacgctgcgtcgtctgggcgacggtaaataggtgttaggcccagagcggcagggtacaact

SEQ ID NO: 70 - RNA, 117 nt, synthetic construct
  source 1..117: mol_type = other RNA
  misc_feature 1: note = EGFP sequence is linked to the 5' end.
  misc_feature 117: note = A poly-A tail is linked to the 3' end.
ctctagaccctgcgtcacagcagcagactgagagagacggattccgtccgcgaattcacgctgcgtcgtctgggcgacggtaaataggtgttaggcccagagcggcagggtacaact

SEQ ID NO: 71 - RNA, 118 nt, synthetic construct
  source 1..118: mol_type = other RNA
  misc_feature 1: note = EGFP sequence is linked to the 5' end.
  misc_feature 118: note = A poly-A tail is linked to the 3' end.
ctctagaccctgcgtcacagcagcagacctgagagagacggattccgtccgcgaattcacgctgcgtcgtctgggcgacggtaaataggtgttaggcccagagcggcagggtacaact

SEQ ID NO: 72 - RNA, 122 nt, synthetic construct
  source 1..122: mol_type = other RNA
  misc_feature 1: note = EGFP sequence is linked to the 5' end.
  misc_feature 122: note = A poly-A tail is linked to the 3' end.
ctctagaccctgcgtcacaaagaaaagacgacgtgagagagacggattccgtccgcgaattcacgctgcgtcgtctgggcgacggtaaataggtgttaggcccagagcggcagggtacaact

SEQ ID NO: 73 - RNA, 115 nt, synthetic construct
  source 1..115: mol_type = other RNA
  misc_feature 115: note = A poly-A tail is linked to the 3' end.
  misc_feature 1: note = EGFP sequence is linked to the 5' end.
ctctagaccctgcgtcacatgagagagacggattccgtccgcgaattcacgctgtgtgacacgtcgtctgggcgacggtaaataggtgttaggcccagagcggcaaggtctaact

SEQ ID NO: 74 - RNA, 115 nt, synthetic construct
  source 1..115: mol_type = other RNA
  misc_feature 1: note = EGFP sequence is linked to the 5' end.
  misc_feature 115: note = A poly-A tail is linked to the 3' end.
ctctagaccctgcgtcacatgagagagacggattccgtccgcgaattcacgctgtgtgacacgtcgtctgggcgacggtaaataggtgttaggcccagagcggcagagtctaact

SEQ ID NO: 75 - DNA, 4450 bp, synthetic construct
  source 1..4450: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttcgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagagagacccagacatcagtggtctctccggagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggctctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

SEQ ID NO: 76 - DNA, 5049 bp, synthetic construct
  source 1..5049: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttcgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgccgagtaacgctgtttctctaacttgtaggaatgaattcagatatttccagagaatgaaaaaaaaatcttcagtagaaggtaatgtgtgagagagacggattccgtccgcgaattcacgctgcacattacgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagcacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagagagacccagacatcagtggtctctccggagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggctctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

SEQ ID NO: 77 - RNA, 46 nt, synthetic construct
  source 1..46: mol_type = other RNA
ggtgtccgcgaattcacgctgcttgttcgcaagtgagagagacacc

SEQ ID NO: 78 - RNA, 36 nt, synthetic construct
  source 1..36: mol_type = other RNA
ccgcgaattcacgctgcttgttcgcaagtgagagag

SEQ ID NO: 79 - RNA, 50 nt, synthetic construct
  source 1..50: mol_type = other RNA
gtaatgtccgcgaattcacgctgcttgttcgcaagtgagagagacattac

SEQ ID NO: 80 - RNA, 52 nt, synthetic construct
  source 1..52: mol_type = other RNA
gtaatgtgccgcgaattcacgctgcttgttcgcaagtgagagagcacattac

SEQ ID NO: 81 - RNA, 54 nt, synthetic construct
  source 1..54: mol_type = other RNA
gtaatgtggccgcgaattcacgctgcttgttcgcaagtgagagagccacattac

SEQ ID NO: 82 - RNA, 70 nt, synthetic construct
  source 1..70: mol_type = other RNA
ggtgtccgcgaattcacgctgcttgttcgcaagtgagagagacaccaaaaaaaaaaaaaaaaaaaaaaaa

SEQ ID NO: 83 - DNA, 36 bp, synthetic construct
  source 1..36: mol_type = other DNA
ccgcgaattcacgctgcttgttcgcaagtgagagag

SEQ ID NO: 84 - DNA, 50 bp, synthetic construct
  source 1..50: mol_type = other DNA
gtaatgtccgcgaattcacgctgcttgttcgcaagtgagagagacattac

SEQ ID NO: 85 - DNA, 52 bp, synthetic construct
  source 1..52: mol_type = other DNA
gtaatgtgccgcgaattcacgctgcttgttcgcaagtgagagagcacattac

SEQ ID NO: 86 - DNA, 54 bp, synthetic construct
  source 1..54: mol_type = other DNA
gtaatgtggccgcgaattcacgctgcttgttcgcaagtgagagagccacattac

(19) United States
(12) Patent Application Publication
(10) Pub. No.: US 2026/0327628 A1
(43) Pub. Date: Oct. 22, 2026

(54) AN RNA APTAMER THAT BINDS TO ASP7967 OR AN ANALOGUE THEREOF

(71) Applicant: OKINAWA INST. OF SCIENCE & TECH. SCHOOL CORPORATION, Okinawa (JP)

(72) Inventors: Yohei YOKOBAYASHI, Okinawa (JP); Venugopal DHAMODHARAN, Okinawa (JP); Keisuke FUKUNAGA, Okinawa (JP); Nao MIYAHIRA, Okinawa (JP); Jiro FUJIYASU, Tsukuba-shi Ibaraki (JP); Shizuma ISHIKAWA, Tsukuba-shi Ibaraki (JP); Yasuaki OOSUMI, Tsukuba-shi Ibaraki (JP)

(73) Assignee: OKINAWA INST. OF SCIENCE & TECH. SCHOOL CORPORATION, Okinawa (JP)

(21) Appl. No.: 19/116,560

(22) PCT Filed: Oct. 6, 2023
(86) PCT No.: PCT/JP2023/036570

§ 371 Date: Mar. 28, 2025

(30) Foreign Application Priority Data
Oct. 7, 2022 (JP) 2022-162289

(51) Int. Cl. C12N 15/115; A61K 31/53; A61K 31/7105

(52) U.S. Cl. CPC C12N 15/115; A61K 31/53; A61K 31/7105

(57) ABSTRACT

It is an object of the present invention to provide an RNA aptamer capable of binding to ASP7967 or an analogue thereof. The RNA aptamer of the present invention is an RNA aptamer that binds to ASP7967 or an analogue thereof, comprising a sequence: -X₁-L₁-X₂-L₂-X₃-, wherein X₁ has the sequence Y₁GY₂GY₃Y₄Y₅; L₁ is a first stem loop nucleotide sequence comprising a first stem region, a first loop region and a second stem region, wherein the first stem region and the second stem region are 2 or more base pair long and are substantially complementary to each other; X₂ is A, G, C or U, L₂ is a second stem loop nucleotide sequence comprising a third stem region, a second loop region and a fourth stem region, wherein the third stem region and the fourth stem region are 2 or more base pair long and are substantially complementary to each other; and wherein the first base in the third stem region is G and the last base in the fourth stem region is C; X₃ has the sequence UY₆; and Y₁, Y₂, Y₃, Y₄, Y₅, and Y₆ are, each independently, A, G, C or U; or a sequence: -S₁-X₂-L₂-X₃-L₃-X₁-S₂-, wherein S₁ and S₂ are, each independently, A, G, C or U, and S₁ and S₂ are capable of forming a base pair or a wobble base pair with each other; L₃ is a third stem loop nucleotide sequence comprising a fifth stem region, a third loop region and a sixth stem region, wherein the fifth stem region and the sixth stem region are 1 or more base pair long and are substantially complementary to each other; and X₁, X₂, X₃, and L₂ are as defined above; or a sequence: -S₃-X₃-L₃-X₁-L₁-X₂-S₄-, wherein S₃ is C and S₄ is G; and X₁, X₂, X₃, L₁, and L₃ are as defined above.

### TECHNICAL FIELD

The present invention is related to an RNA aptamer that binds to ASP7967 or an analogue thereof.

### BACKGROUND ART

Chemical regulation of gene expression by a small molecule enables precise control over the timing and level of gene expression in mammalian cells¹. Such gene switches are highly valuable for basic research as well as for practical applications such as metabolic engineering and gene/cell therapies². While conventional gene switches based on protein transcription factors such as Tet-ON and Tet-OFF systems exhibit excellent gene regulation^{3,4}, there are a number of inherent drawbacks for many applications such as immunogenicity of the exogenous protein components, large genetic size, and the need to use an engineered promoter.

Alternatively, riboswitches have emerged as a new class of gene switches that do not rely on exogenous protein factors⁵⁻⁷. These riboswitches typically employ an RNA aptamer that specifically binds a small molecule. A structural change often accompanied by aptamer-ligand binding is exploited to regulate gene expression through various mechanisms⁸.

### SUMMARY OF INVENTION

### Technical Problem

While RNA aptamers that bind various small molecules can be selected in vitro from random RNA sequences by systematic evolution of ligands by exponential enrichment (SELEX)⁹, such aptamers do not always function in cellular environment or as a part of riboswitches. Consequently, small molecules and their cognate aptamers that have been used to design mammalian riboswitches are rather limited¹⁰. Theophylline and tetracycline, and their aptamers discovered by SELEX have been used extensively in synthetic mammalian riboswitches¹¹⁻²². Similarly, we and others have used a natural aptamer from a bacterial guanine-responsive riboswitch to make mammalian riboswitches^{14,19,20,23-32}. More recently, ciprofloxacin, hypoxanthine, (6R,S)-folinic acid, and cyclic di-GMP, and their aptamers have been used to construct mammalian riboswitches^{16,33}. It should be noted that most of these riboswitches require relatively high concentrations (~100 μM or higher) of the small molecule to achieve maximum gene regulation^{16,20,33}.

Consequently, additional small molecules and aptamers that function in mammalian cells are needed to extend the applications of riboswitches.

Therefore, an object of the present invention is to provide an RNA aptamer capable of binding to ASP7967 or an analogue thereof.

### Solution to Problem

To solve the above-mentioned problem, we have made an exploration of novel aptamer-ligand pairs compatible with applications in mammalian cells. We discovered an RNA aptamer AC17-4 that binds a previously reported small molecule ASP2905³⁴⁻³⁶ and its analog ASP7967 (FIG. 2a) with a dissociation constant (K_{D}) of ~50 nM at 37° C. The aptamer was combined with the recently reported self-cleaving ribozyme scaffold (circularly-permuted pistol, CPP)²⁷ to create riboswitches that activate gene expression in response to the small molecules in HEK293 cells. A riboswitch was then incorporated into an adeno-associated virus serotype 8 (AAV8) vector expressing human erythropoietin (hEPO) which was used to regulate hEPO expression in vivo using orally administered ASP7967.

Accordingly the present invention relates to the following:
(1) An RNA aptamer that binds to ASP7967 or an analogue thereof, the aptamer comprising a sequence:

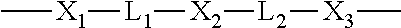

wherein
   X₁ has the sequence Y₁GY₂GY₃Y₄Y₅,
   L₁ is a first stem loop nucleotide sequence comprising a first stem region, a first loop region and a second stem region, wherein the first stem region and the second stem region are 2 or more base pair long and are substantially complementary to each other;
   X₂ is A, G, C or U,
   L₂ is a second stem loop nucleotide sequence comprising a third stem region, a second loop region and a fourth stem region, wherein the third stem region and the fourth stem region are 2 or more base pair long and are substantially complementary to each other; and wherein the first base in the third stem region is G and the last base in the fourth stem region is C;
   X₃ has the sequence UY₆; and
   Y₁, Y₂, Y₃, Y₄, Y₅, and Y₆ are, each independently, A, G, C or U; or
a sequence:

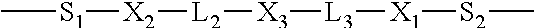

wherein
   S₁ and S₂ are, each independently, A, G, C or U, and S₁ and S₂ are capable of forming a base pair or a wobble base pair with each other;
   L₃ is a third stem loop nucleotide sequence comprising a fifth stem region, a third loop region and a sixth stem region, wherein the fifth stem region and the sixth stem region are 1 or more base pair long and are substantially complementary to each other; and
   X₁, X₂, X₃, and L₂ are as defined above; or
a sequence:

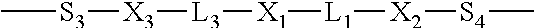

wherein
   S₃ is C and S₄ is G; and
   X₁, X₂, X₃, L₁, and L₃ are as defined above.
(2) The RNA aptamer described in the above-mentioned (1),
   wherein
   Y₂ is selected from A or U;
   Y₃ is selected from A or U; and/or
   Y₄ is selected from G or C.
(3) The RNA aptamer described in the above-mentioned (2),
   wherein
   Y₂ is A;
   Y₃ is A; and/or
   Y₄ is G.
(4) The RNA aptamer described in the above-mentioned (1),
   wherein
   Y₁ and Y₆ are capable of forming a base pair or a wobble base pair with each other.
(5) The RNA aptamer described in the above-mentioned (1),
   wherein
   Y₁ is G and Y₆ is U; or
   Y₁ is U and Y₆ is G.
(6) The RNA aptamer described in the above-mentioned (1),
   wherein
   the first stem region and the second stem region are 3 to 7 base pair long and are substantially complementary to each other.
(7) The RNA aptamer described in the above-mentioned (1),
   wherein
   the first stem region and the second stem region are 5 base pair long and are substantially complementary to each other.
(8) The RNA aptamer described in the above-mentioned (1),
   wherein
   the first stem region has the sequence GACGG and the second stem region has the sequence CCGUC.
(9) The RNA aptamer described in the above-mentioned (1),
   wherein
   the first loop region has 3 to 7 bases.
(10) The RNA aptamer described in the above-mentioned (1),
   wherein
   the first loop region has the sequence AUU or UUCG.
(11) The RNA aptamer described in the above-mentioned (1),
   wherein
   the third stem region and the fourth stem region have 1 to 5 base pairs and are substantially complementary to each other.
(12) The RNA aptamer described in the above-mentioned (1),
   wherein
   the third stem region and the fourth stem region have 3 or 4 base pairs and are substantially complementary to each other.
(13) The RNA aptamer described in the above-mentioned (1),
   wherein
   the third stem region has the sequence GCG and the fourth stem region has the sequence CGC; or
the third stem region has the sequence GCGU and the fourth stem region has the sequence ACGC.
(14) The RNA aptamer described in the above-mentioned (1),
   wherein
   the second loop region has 3 to 7 bases.
(15) The RNA aptamer described in the above-mentioned (1),
   wherein
   the second loop region has the sequence AAUUCA or UUCG.
(16) The RNA aptamer described in the above-mentioned (1), wherein the RNA aptamer comprises the sequence: -X₁-L₁-X₂-L₂-X₃—, and further comprises a fifth stem region adjacent to the 5′ terminus of X₁ and a sixth stem region adjacent to the 3′ terminus of X₃, wherein the fifth stem region and the sixth stem region have 1 to 15 base pairs and are substantially complementary to each other, and wherein the fifth stem region and the sixth stem region form a double-stranded stem.
(17) The RNA aptamer described in the above-mentioned (1),
   wherein
   the fifth stem region and the six stem region are 3 to 7 base pair long and are substantially complementary to each other.
(18) The RNA aptamer described in the above-mentioned (1),
   wherein
   the fifth stem region and the six stem region are 4 base pair long and are substantially complementary to each other.
(19) The RNA aptamer described in the above-mentioned (1),
   wherein
   the fifth stem region has the sequence CUUG and the sixth stem region has the sequence CAAG.
(20) The RNA aptamer described in the above-mentioned (1),
   wherein
   the third loop region has 3 to 7 bases.
(21) The RNA aptamer described in the above-mentioned (1),
   wherein
   the third loop region has the sequence UUCG.
(22) The RNA aptamer described in the above-mentioned (1),
   wherein
   S₁ is C and S₂ is G; or
   S₁ is G and S₂ is C.
(23) The RNA aptamer described in the above-mentioned (1), wherein the RNA aptamer comprises the sequence: -S₁-X₂-L₂-X₃-L₃-X₁-S₂—, and further comprises a seventh stem region adjacent to the 5′ terminus of S₁ and a eighth stem region adjacent to the 3′ terminus of S₂, wherein the seventh stem region and the eighth stem region have 1 to 15 base pairs and are substantially complementary to each other, and wherein the seventh stem region and the eighth stem region form a double-stranded stem.
(24) The RNA aptamer described in the above-mentioned (1), wherein the RNA aptamer comprises the sequence: -S₃-X₃-L₃-X₁-L₁-X₂-S₄—, and further comprises a ninth stem region adjacent to the 5′ terminus of S₃ and a tenth stem region adjacent to the 3′ terminus of S₄, wherein the ninth stem region and the tenth stem region have 1 to 15 base pairs and are substantially complementary to each other, and wherein the ninth stem region and the tenth stem region form a double-stranded stem.
(25) The RNA aptamer described in the above-mentioned (1),
   wherein the RNA aptamer is circularly permuted.
(26) The RNA aptamer described in the above-mentioned (1),
   wherein the analogue of ASP7967 is ASP2905.
(27) An RNA or DNA vector comprising the RNA aptamer described in the above-mentioned (1) or a DNA sequence that is capable of being transcribed into the RNA aptamer described in the above-mentioned (1).
(28) A riboswitch comprising the RNA aptamer described in the above-mentioned (1).
(29) An RNA or DNA vector comprising the riboswitch described in the above-mentioned (28) or a DNA sequence that is capable of being transcribed into the riboswitch described in the above-mentioned (28).
(30) The RNA or DNA vector described in the above-mentioned (29), further comprising a target sequence operably liked to the riboswitch or the DNA sequence, wherein the target sequence encodes a protein; or wherein the target sequence is either an siRNA, pre-miRNA, pri-miRNA, sgRNA, lncRNA, RNA aptamer, ribozyme, RNA, or rRNA; or a DNA sequence that is capable of being transcribed into an siRNA, pre-miRNA, pri-miRNA, sgRNA, lncRNA, RNA aptamer, ribozyme, tRNA, or rRNA.
(31) An isolated polynucleotide comprising:
   a riboswitch comprising an RNA aptamer capable of binding to ASP7967 or an analogue thereof, or a DNA sequence that is capable of being transcribed into the riboswitch and
   a target sequence encoding a protein
   wherein the riboswitch is operably linked to the target sequence such that expression of the protein is upregulated or downregulated in response to ASP7967 or the analogue thereof.
(32) The polynucleotide described in the above-mentioned (31), wherein the target sequence comprises a plurality of exons.
(33) The polynucleotide described in the above-mentioned (32), wherein the target sequence comprises an alternatively-spliced exon, flanked by a 5′ intron and a 3′ intron,
   wherein the alternatively-spliced exon comprises a stop codon that is in-frame with the protein when the alternatively-spliced exon is spliced into an mRNA of the protein.
(34) The polynucleotide described in the above-mentioned (31), wherein the polynucleotide further comprises 3′ UTR comprising a polyadenylation signal sequence, and wherein the riboswitch is inserted within the 3′ UTR and at the 5′ side of the polyadenylation signal sequence, and wherein the function of the polyadenylation signal sequence is regulated by the riboswitch.
(35) The polynucleotide described in the above-mentioned (34), wherein the riboswitch further comprises a self-cleaving ribozyme.
(36) The polynucleotide described in the above-mentioned (35), wherein the self-cleaving ribozyme is activated when the aptamer binds to ASP7967 or an analogue thereof or wherein the self-cleaving ribozyme is inactivated when the aptamer binds to ASP7967 or an analogue thereof.
(37) A kit for regulating expression of a protein, comprising
   ASP7967 or an analogue thereof, and
the polynucleotide described in the above-mentioned (31) or a vector comprising the polynucleotide described in the above-mentioned (31).
(38) The kit described in the above-mentioned (37),
   wherein the kit is for treating a disease.
(39) The kit described in the above-mentioned (38),
   wherein the disease is a central nervous system disease, a cognitive disorder, or a KCNH₃-related disease.
(40) The kit described in the above-mentioned (39),
   wherein the disease is ADHD, Parkinson's disease, Alzheimer's disease, or schizophrenia.
(41) A method for regulating expression of a protein in vivo, comprising
   introducing the polynucleotide described in the above-mentioned (31) or a vector comprising the polynucleotide described in the above-mentioned (31) into a cell, and
   bringing ASP7967 or an analogue thereof into contact with the polynucleotide or the vector.
(42) A method for treating or preventing a disease, comprising
   introducing the polynucleotide described in the above-mentioned (31) or a vector comprising the polynucleotide described in the above-mentioned (31) into a subject, and
   administering ASP7967 or an analogue thereof into the subject.
(43) The method described in the above-mentioned (42),
   wherein the disease is a central nervous system disease, a cognitive disorder, or a KCNH₃-related disease.
(44) The method described in the above-mentioned (43),
   wherein the disease is ADHD, Parkinson's disease, Alzheimer's disease, or schizophrenia.
(45) A method for treating a disease, comprising:
   administering ASP7967 or an analogue thereof to a subject who have received a gene therapy with a vector comprising the polynucleotide described in the above-mentioned (31).
(46) The method described in the above-mentioned (45),
   wherein the target sequence encodes a protein selected from the group consisting of:
   4-1BB ligand, 5-helix, human C-C chemokine, human L105 chemokine, human L105 chemokine designated huL105_3., monokine induced by gamma-interferon (MIG), partial CXCR4B protein, platelet basic protein (PBP), α1-antitrypsin, ACRP-30 Homologue; Complement Component Clq C, Adenoid-expressed chemokine (ADEC), aFGF; FGF-1, AGF, AGF Protein, albumin, an etoposide, angiostatin, Anthrax vaccine, Antibodies specific for collapsin, antistasin, Anti-TGF beta family antibodies, antithrombin III, APM-1; ACRP-30; Famoxin, apo-lipoprotein species, Arylsulfatase B, b57 Protein, BCMA, Beta-thromboglobulin protein (beta-TG), bFGF; FGF2, Blood coagulation factors, BMP Processing Enzyme Furin, BMP-10, BMP-12, BMP-15, BMP-17, BMP-18, BMP-2B, BMP-4, BMP-5, BMP-6, BMP-9, Bone Morphogenic Protein-2, calcitonin, Calpain-10a, Calpain-10b, Calpain-10c, Cancer Vaccine, Carboxypeptidase, C-C chemokine, MCP2, CCR5 variant, CCR7, CCR7, CD11a Mab, CD137; 4-1BB Receptor Protein, CD20 Mab, CD27, CD27L, CD30, CD30 ligand, CD33 immunotoxin, CD40, CD40L, CD52 Mab, Cerebus Protein, Chemokine Eotaxin., Chemokine hIL-8, Chemokine hMCP1, Chemokine hMCP1a, Chemokine hMCP1b, Chemokine hMCP2, Chemokine hMCP3, Chemokine hSDF1b, Chemokine MCP-4, chemokine TECK and TECK variant, Chemokine-like protein IL-8M1 Full-Length and Mature, Chemokine-like protein IL-8M10 Full-Length and Mature, Chemokine-like protein IL-8M3, Chemokine-like protein IL-8M8 Full-Length and Mature, Chemokine-like protein IL-8M9 Full-Length and Mature, Chemokine-like protein PF4-414 Full-Length and Mature, Chemokine-like protein PF4-426 Full-Length and Mature, Chemokine-like protein PF4-M2 Full-Length and Mature, Cholera vaccine, Chondromodulin-like protein, c-kit ligand; SCF; Mast cell growth factor; MGF; Fibrosarcoma-derived stem cell factor, CNTF and fragment thereof, coagulation factors in both pre and active forms, collagens, Complement C5 Mab, Connective tissue activating protein-III, CTAA16.88 Mab, CTAP-III, CTLA4-Ig, CTLA-8, CXC3, CXC3, CXCR3; CXC chemokine receptor 3, cyanovirin-N, Darbepoetin, designated exodus, designated huL105_7., DIL-40, Dnase, EDAR, EGF Receptor Mab, ENA-78, Endostatin, Eotaxin, Epithelial neutrophil activating protein-78, EPO receptor; EPOR, erythropoietin (EPO) and EPO mimics, Eutropin, Exodus protein, Factor IX, Factor VII, Factor VIII, Factor X and Factor XIII, FAS Ligand Inhibitory Protein (DcR3), FasL, FasL, FasL, FGF, FGF-12; Fibroblast growth factor homologous factor-1, FGF-15, FGF-16, FGF-18, FGF-3; INT-2, FGF-4; gelonin, HST-1; HBGF-4, FGF-5, FGF-6; Heparin binding secreted transforming factor-2, FGF-8, FGF-9; Glia activating factor, fibrinogen, flt-1, flt-3 ligand, Follicle stimulating hormone Alpha subunit, Follicle stimulating hormone Beta subunit, Follitropin, Fractalkine, fragment. myofibrillar protein Troponin I, FSH, Galactosidase, Galectin-4, G-CSF, GDF-1, Gene therapy, Glioma-derived growth factor, glucagon, glucagon-like peptides, Glucocere-brosidase, glucose oxidase, Glucosidase, Glycodelin-A; Progesterone-associated endometrial protein, GM-CSF, gonadotropin, Granulocyte chemotactic protein-2 (GCP-2), Granulocyte-macrophage colony stimulating factor, growth hormone, Growth related oncogene-alpha (GRO-alpha), Growth related oncogene-beta (GRO-beta), Growth related oncogene-gamma (GRO-gamma), hAPO-4; TROY, hCG, Hepatitus B surface Antigen, Hepatitus B Vaccine, HER2 Receptor Mab, hirudin, HIV gp120, HIV gp41, HIV Inhibitor Peptide, HIV Inhibitor Peptide, HIV Inhibitor Peptide, HIV protease inhibiting peptides, HIV-1 protease inhibitors, HPV vaccine, Human 6CKine protein, Human Act-2 protein, Human adipogenesis inhibitory factor, human B cell stimulating factor-2 receptor, Human beta-chemokine H1305 (MCP-2), Human C-C chemokine DGWCC, Human CC chemokine ELC protein, Human CC type chemokine interleukin C, Human CCC3 protein, Human CCF18 chemokine, Human CC-type chemokine protein designated SLC (secondary lymphoid chemokine), Human chemokine beta-8 short forms, Human chemokine C10, Human chemokine CC-2, Human chemokine CC-3, Human chemokine CCR-2, Human chemokine Ckbeta-7, Human chemokine ENA-78, Human chemokine eotaxin, Human chemokine GRO alpha, Human chemokine GROalpha, Human chemokine GRObeta, Human chemokine HCC-1, Human chemokine HCC-1, Human chemokine 1-309, Human chemokine IP-10, Human chemokine L105_3, Human chemokine L105_7, Human chemokine MIG, Human chemokine MIG-beta protein, Human chemokine MIP-1 alpha, Human chemokine MIP1beta, Human chemokine MIP-3alpha, Human chemokine MIP-3beta, Human chemokine PF4, Human chemokine protein 331D5, Human chemokine protein 61164, Human chemokine receptor CXCR3, Human chemokine SDF1alpha, Human chemokine SDF1beta, Human chemokine ZSIG-35, Human Chr19Kine protein, Human CKbeta-9, Human CKbeta-9, Human CX3C 111 amino acid chemokine, Human DNAX interleukin-40, Human DVic-1 C-C chemokine, Human EDIRF I protein sequence, Human EDIRF II protein sequence, Human eosinocyte CC type chemokine eotaxin, Human eosinophil-expressed chemokine (EEC), Human fast twitch skeletal muscle troponin C, Human fast twitch skeletal muscle troponin I, Human fast twitch skeletal muscle Troponin subunit C, Human fast twitch skeletal muscle Troponin subunit I Protein, Human fast twitch skeletal muscle Troponin subunit T, Human fast twitch skeletal muscle troponin T, Human foetal spleen expressed chemokine, FSEC, Human GM-CSF receptor, Human gro-alpha chemokine, Human gro-beta chemokine, Human gro-gamma chemokine, Human IL-16 protein, Human IL-1RD10 protein sequence, Human IL-1RD9, Human IL-5 receptor alpha chain, Human IL-6 receptor, Human IL-8 receptor protein hIL8RA, Human IL-8 receptor protein hIL8RB, Human IL-9 receptor protein, Human IL-9 receptor protein variant #3, Human IL-9 receptor protein variant fragment, Human IL-9 receptor protein variant fragment #3, Human interleukin 1 delta, Human Interleukin 10, Human Interleukin 10, Human interleukin 18, Human interleukin 18 derivatives, Human interleukin-1 beta precursor, Human interleukin-1 beta precursor, Human interleukin-1 receptor accessory protein, Human interleukin-1 receptor antagonist beta, Human interleukin-1 type-3 receptor, Human Interleukin-10 (precursor), Human Interleukin-10 (precursor), Human interleukin-11 receptor, Human interleukin-12 40 kD subunit, Human interleukin-12 beta-1 receptor, Human interleukin-12 beta-2 receptor, Human Interleukin-12 p35 protein, Human Interleukin-12 p40 protein, Human interleukin-12 receptor, Human interleukin-13 alpha receptor, Human interleukin-13 beta receptor, Human interleukin-15, Human interleukin-15 receptor from clone P1, Human interleukin-17 receptor, Human interleukin-18 protein (IL-18), Human interleukin-3, human interleukin-3 receptor, Human interleukin-3 variant, Human interleukin-4 receptor, Human interleukin-5, Human interleukin-6, Human interleukin-7, Human interleukin-7, Human interleukin-8 (IL-8), Human intracellular IL-1 receptor antagonist, Human IP-10 and HIV-1 gp120 hypervariable region fusion protein, Human IP-10 and human Muc-1 core epitope (VNT) fusion protein, human liver and activation regulated chemokine (LARC), Human Lkn-1 Full-Length and Mature protein, Human mammary associated chemokine (MACK) protein Full-Length and Mature, Human mature chemokine Ckbeta-7, Human mature gro-alpha, Human mature gro-gamma polypeptide used to treat sepsis, Human MCP-3 and human Muc-1 core epitope (VNT) fusion protein, Human MI10 protein, Human MILA protein, Human monocyte chemoattractant factor hMCP-1, Human monocyte chemoattractant factor hMCP-3, Human monocyte chemotactic proprotein (MCPP) sequence, Human neurotactin chemokine like domain, Human non-ELR CXC chemokine H174, Human non-ELR CXC chemokine IP10, Human non-ELR CXC chemokine Mig, Human PAI-1 mutants, Human protein with IL-16 activity, Human protein with IL-16 activity, Human secondary lymphoid chemokine (SLC), Human SISD protein, Human STCP-1, Human stromal cell-derived chemokine, SDF-1, Human T cell mixed lymphocyte reaction expressed chemokine (TMEC), Human thymus and activation regulated cytokine (TARC), Human thymus expressed, Human TNF-alpha, Human TNF-alpha, Human TNF-beta (LT-alpha), Human type CC chemokine eotaxin 3 protein sequence, Human type II interleukin-1 receptor, Human wild-type interleukin-4 (hIL-4) protein, Human ZCHEMO-8 protein, Humanized Anti-VEGF Antibodies, and fragments thereof, Humanized Anti-VEGF Antibodies, and fragments thereof, Hyaluronidase, ICE 10 kD subunit, ICE 20 kD subunit, ICE 22 kD subunit, Iduronate-2-sulfatase, Iduronidase, IL-1 alpha, IL-1 beta, IL-1 inhibitor (IL-1i), IL-1 mature, IL-10 receptor, IL-11, IL-11, IL-12 p40 subunit, IL-13, IL-14, IL-15, IL-15 receptor, IL-17, IL-17 receptor, II-17 receptor, II-17 receptor, IL-19, IL-1i fragments, IL1-receptor antagonist, IL-21 (TIF), IL-3 containing fusion protein, IL-3 mutant proteins, IL-3 variants, IL-3 variants, IL-4, IL-4 mutein, IL-4 mutein Y124G, IL-4 mutein Y124X, IL-4 muteins, II-5 receptor, IL-6, II-6 receptor, IL-7 receptor clone, IL-8 receptor, IL-9 mature protein variant (Met117 version), immunoglobulins or immunoglobulin-based molecules or fragment of either (e.g. a Small Modular ImmunoPharmaceutical™ (“SMIP”) or dAb, Fab′ fragments, F(ab′) 2, scAb, scFv or scFv fragment), including but not limited to plasminogen, Influenza Vaccine, Inhibin alpha, Inhibin beta, insulin, insulin-like growth factor, Integrin Mab, inter-alpha trypsin inhibitor, inter-alpha trypsin inhibitor, Interferon gamma-inducible protein (IP-10), interferons (such as interferon alpha species and sub-species, interferon beta species and sub-species, interferon gamma species and sub-species), interferons (such as interferon alpha species and sub-species, interferon beta species and sub-species, interferon gamma species and sub-species), Interleukin 6, Interleukin 8 (IL-8) receptor, Interleukin 8 receptor B, Interleukin-1alpha, Interleukin-2 receptor associated protein p43, interleukin-3, interleukin-4 muteins, Interleukin-8 (IL-8) protein, interleukin-9, Interleukin-9 (IL-9) mature protein (Thr117 version), interleukins (such as IL0, IL11 and IL2), interleukins (such as IL0, IL11 and IL2), Japanese enc ephalitis vaccine, Kalikrein Inhibitor, Keratinocyte growth factor, Kunitz domain protein (such as aprotinin, amyloid precursor protein and those described in WO 03/066824, with or without albumin fusions), Kunitz domain protein, protinin, amyloid precursor protein with or without albumin fusions, LACI, lactoferrin, Latent TGF-beta binding protein II, leptin, Liver expressed chemokine-1 (LVEC-1), Liver expressed chemokine-2 (LVEC-2), LT-alpha, LT-beta, Luteinization Hormone, Lyme Vaccine, Lymphotactin, Macrophage derived chemokine analogue MDC (n+1), Macrophage derived chemokine analogue MDC-eyfy, Macrophage derived chemokine analogue MDC-yl, Macrophage derived chemokine, MDC, Macrophage-derived chemokine (MDC), Maspin; Protease Inhibitor 5, MCP-1 receptor, MCP-1a, MCP-1b, MCP-3, MCP-4 receptor, M-CSF, Melanoma inhibiting protein, Membrane-bound proteins, Met117 human interleukin 9, MIP-3 alpha, MIP-3 beta, MIP-Gamma, MIRAP, Modified Rantes, monoclonal antibody, MP52, Mutant Interleukin 6 S176R, myofibrillar contractile protein Troponin I, Natriuretic Peptide, Nerve Growth Factor-beta, Nerve Growth Factor-beta2, Neuropilin-1, Neuropilin-2, Neurotactin, Neu-rotrophin-3, Neurotrophin-4, Neurotrophin-4a, Neurotrophin-4b, Neurotrophin-4c, Neurotrophin-4d, Neutrophil activating peptide-2 (NAP-2), NOGO-66 Receptor, NOGO-A, NOGO-B, NOGO-C, Novel beta-chemokine designated PTEC, N-terminal modified chemokine GroHEK/hSDF-1alpha, N-terminal modified chemokine GroHEK/hSDF-1beta, N-terminal modified chemokine met-hSDF-1 alpha, N-terminal modified chemokine met-hSDF-1 beta, OPGL, Osteogenic Protein-1; OP-1; BMP-7, Osteogenic Protein-2, OX40; ACT-4, OX40L, Oxytocin (Neurophysin I), parathyroid hormone, Patched, Patched-2, PDGF-D, Pertussis toxoid, Pituitary expressed chemokine (PGEC), Placental Growth Factor, Placental Growth Factor-2, Plasminogen Activator Inhibitor-1; PAI-1, Plasminogen Activator Inhibitor-2; PAI-2, Plasminogen Activator Inhibitor-2; PAI-2, Platelet derived growth factor, Platelet derived growth factor Bv-sis, Platelet derived growth factor precursor A, Platelet derived growth factor precursor B, Platelet Mab, platelet-derived endothelial cell growth factor (PD-ECGF), Platelet-Derived Growth Factor A chain, Platelet-Derived Growth Factor B chain, polypeptide used to treat sepsis, Preproapolipoprotein “milano” variant, Pre-proapolipoprotein “paris” variant, pre-thrombin, Primate CC chemokine “ILINCK”, Primate CXC chemokine “IBICK”, proinsulin, Prolactin, Prolactin2, prosaptide, Protease inhibitor peptides, Protein C, Protein S, pro-thrombin, prourokinase, RANTES, RANTES 8-68, RANTES 9-68, RANTES peptide, RANTES receptor, Recombinant interleukin-16, Resistin, restrictocin, Retroviral protease inhibitors, ricin, Rotavirus Vaccine, RSV Mab, saporin, sarcin, Secreted and Transmembrane polypeptides, Secreted and Transmembrane polypeptides, serum cholinesterase, serum protein, blood clotting factor, Soluble BMP Receptor Kinase Protein-3, Soluble VEGF Receptor, Stem Cell Inhibitory Factor, Straphylococcus Vaccine, Stromal Derived Factor-1 alpha, Stromal Derived Factor-1 beta, Substance P (tachykinin), T1249 peptide, T20 peptide, T4 Endonuclease, TACI, Tarc, TGF-beta 1, TGF-beta 2, Thr117 human interleukin 9, thrombin, thrombopoietin, Thrombopoietin derivative1, Thrombopoietin derivative2, Thrombopoietin derivative3, Thrombopoietin derivative4, Thrombopoietin derivative5, Thrombopoietin derivative6, Thrombopoietin derivative7, Thymus expressed chemokine (TECK), Thyroid stimulating Hormone, tick anticoagulant peptide, Tim-1 protein, TNF-alpha precursor, TNF-R, TNF-RII; TNF p75 Receptor; Death Receptor, tPA, transferrin, transforming growth factor beta, Troponin peptides, Truncated monocyte chemotactic protein 2 (6-76), Truncated monocyte chemotactic protein 2 (6-76), Truncated RANTES protein (3-68), tumour necrosis factor, Urate Oxidase, urokinase, Vasopressin (Neurophysin II), VEGF R-3; flt-4, VEGF Receptor; KDR; flk-1, VEGF-110, VEGF-121, VEGF-138, VEGF-145, VEGF-162, VEGF-165, VEGF-182, VEGF-189, VEGF-206, VEGF-D, VEGF-E; VEGF-X, von Willebrand's factor, Wild type monocyte chemotactic protein 2, Wild type monocyte chemotactic protein 2, ZTGF-beta 9, β^{(T870)-}globin, SMN1, chimeric antigen receptors, RPE65, F8, HGF, LPL, p53, apoe2, Arylsulfatase A, NAGLU, SGSH, AADC, GAD, GDNF, NRTN, LCAT, GBA, FGF-1, FGF-2, ADA, CLN2, CLN6, CLN3, IDS, Huntingtin, TRAIL, dystrophin, GALGT2, accA, IDUA, GLB1, FS344, SGCA, DYSF, ABCD1, Gigaxonin and functional fragments thereof.

Incidentally, each constitution of the above-mentioned (1) to (44) can be combined by arbitrary selecting two or more.

### Advantageous Effects of the Invention

The RNA aptamer of the present invention has a superior binding force to ASP7967 or an analogue thereof. Therefore, for example, the RNA aptamer of the present invention is very useful for designing a riboswitch which can sensitively respond to the existence of ASP7967 or an analogue thereof.

### BRIEF DESCRIPTION OF DRAWINGS

FIG. 1a: ¹H NMR spectrum of compound 3. FIG. 1b: ¹³C NMR spectrum of compound 3. FIG. 1c: ¹⁹F NMR spectrum of compound 3.

FIG. 2 Small molecules ASP2905/ASP7967 and aptamer. a, Structures of ASP2905, ASP7967, and compound 3. b, Aptamer sequence and predicted structures. R10-6 is the original aptamer discovered by SELEX. AC17-4 was extracted from the predicted structure of R10-6 that was found to be sufficient for binding ASP2905 and ASP7967. The predicted structures are based on mFold38. c, SPR sensorgrams of AC17-4-immobilized chip injected with ASP2905 and ASP7967 solutions.

FIG. 3 ITC measurement of AC17-4 and ASP2905. Measurement was performed at 37° C. The data are mean±standard deviation (S.D.) of two independent experiments.

FIG. 4 AC17-4 mutational analysis. a, Mutations and their effect on K_{D} for ASP2905. Mean values in parentheses represent K_{D} as measured by SPR. NB: no binding. b, Summary of the K_{D} values of the mutants depicted in a. The SPR sensorgrams are provided in FIG. 5.

FIG. 5 SPR sensorgrams of AC17-4 mutants interacting with ASP2905. These measurements were used to produce data shown in FIG. 4. The K_{D} values are mean of two independent experiments.

FIG. 6 Mammalian riboswitches based on AC17-4. a, Sequence and secondary structure of a pistol ribozyme. Arrowhead indicates the cleavage site. b, AC17-4-CPP aptazyme embedded in the 3′ UTR of an EGFP mRNA. In the absence of the ligand (ASP2905 or ASP7967), the active CPP ribozyme self-cleaves at the position indicated by the arrowhead (left structure). This results in detachment of the poly(A) sitail and repressed EGFP expression (OFF). In the presence of the ligand, the aptamer-ligand interaction stabilizes the aptazyme structure depicted on the right in which the anti-Rz sequence (circles with black edges) invades the P1 stem and the PK pseudoknot. This interferes with the CPP structure making the ribozyme inactive, and the mRNA is translated (ON). c, Induction of gene expression by the riboswitches depicted in b. The variants with different sizes of the anti-Rz sequence were examined. Empty: noaptazyme control. The data are averages of three replicate wells with the error bars representing S.D. Numbers above the bars indicate ON/OFF ratio. d, Dose-dependent response of a8-AC17-4-CPP in response to ASP7967. Empty: no-aptazyme control. The data are averages of three replicate wells with the error bars representing S.D. Numbers near the data points indicate ON/OFF ratio.

FIG. 7 MTT (cell proliferation) assay of HEK293 cells cultured in the presence of ASP2095 or ASP7967. No cellular toxicity up to 10 μM was observed. The data are averages of four replicate wells with the error bars representing S.D. Statistical comparison was performed by unpaired two-tailed t-test. ns: not significant.

FIG. 8 Riboswitch function of a8c-AC17-4-CPP in HEK293 cells. a, Sequence and secondary structure of a8c-AC17-4-CPP. b, The experimental conditions were the same as those shown in FIG. 6c. Empty: no-aptazyme control. The data are averages of three replicate wells with the error bars representing S.D. Number above each bar indicates ON/OFF ratio.

FIG. 9 Evaluation of a8c-AC17-4 CPP riboswitch function in an AAV vector. a, AAV vector expressing hEPO regulated by a8c-AC17-4-CPP riboswitch. b, hEPO secretion from HEK293 cells transfected with AAV vector plasmids in the absence or presence of ASP7967. Control: no-riboswitch. The data are mean value of three biological replicates with the error bars representing S.D. c, The experimental design used in animal studies. Blood was collected 24 h before and at multiple time points (2 h, 4 h, 6 h, 8 h, 24 h) after the ligand administration. d, Time-dependent secretion of hEPO in mice injected with 3×10¹⁰ vector genomes (vg) per mouse after oral administration of ASP7967. Serum hEPO protein concentrations were measured at the indicated time points. Mice administered saline and vehicle or ASP7967 (WT_vehicle or WT_ASP7967, n=5), AAV8-hEPO-control and vehicle or ASP7967 (AAV8-hEPO-control_vehicle or AAV8-hEPO-control_ASP7967, n=6), AAV8-hEPO-a8c-AC17-4-CPP and vehicle (AAV8-hEPO-a8c-AC17-4-CPP_vehicle, n=7), AAV8-hEPO-a8c-AC17-4-CPP and ASP7967 (AAV8-hEPO-a8c-AC17-4-CPP_ASP7967, n=8). The data are mean value of biological replicates (n) with error bars representing S.E.

FIG. 10 Pharmacokinetics of ASP7967 in mice after oral administration. After oral administration at 100 mg kg⁻¹ to mice (BALB/c cAJc1), plasma and liver concentrations of ASP7967 were measured using liquid chromatography-tandem mass spectrometry (LC-MS/MS). ASP7967 concentrations in both plasma and liver reached maxima within the first sampling time point of 1 h and then decreased over time. The concentration ratio of liver to plasma ranged from 2.7 to 30.6 suggesting that ASP7967 was distributed to the liver.

FIG. 11 Exon-skipping riboswitches based on AC17-4. a, Illustration of the exon-skipping riboswitch mechanism. A suicide exon containing a stop codon flanked by two intron sequences derived from intron 2 of the human β-globin gene is inserted into EGFP gene. AC17-4 aptamer is inserted downstream of the 5′-ss of the second intron. In the absence of the ligand, the suicide exon is incorporated into the mature mRNA. In the presence of the ligand, the 5′-ss is masked by the aptamer structure and results in exon skipping. This allows the desired protein to be expressed. b, EGFP expression regulated by the exon-skipping riboswitches with varying P1 stability. c, EGFP expression regulated by exon-skipping/aptazyme dual riboswitches. b-c, Empty: noaptazyme control. The data are averages of three replicate wells with the error bars representing S.D. Number above each bar indicates ON/OFF ratio.

FIG. 12 Sequences and secondary structures of CPP-4a9-P3-9d and CPP-4a9-P3-9e aptazymes.

FIG. 13 The map and sequence of the plasmid pEGFP-BsaI-Amp. Riboswitch sequence shown in Table 5 replaces the sequence shown in underlined bold in the corresponding riboswitch plasmid. CMV promoter is shown in box. egfp gene is shown in shading. bla gene (Amp^{R}) is shown in gray. The plasmid map based on Benchling is available from the link below: https://benchling.com/s/seq-rsh62LI9bm2tWJWB2Ecm?m=slm-aMKjz51vOZsXH6yS C4ih

FIG. 14 The map and sequence of the plasmid pEGFP-ex169-AC17-4-a8. Riboswitch sequence shown in Table 6 replaces the sequence shown in underlined bold part in the corresponding riboswitch plasmid. In the dual exon-skipping/aptazyme switches (a9+g2g7/CPP-4a9-P3-9d and a9+g2g7/CPP-4a9-P3-9e), the corresponding aptazyme sequence shown in Table 5 replaces the sequence shown in bold/box in addition to the ex169-AC17-4-a9+g2g7 exon-skipping module. CMV promoter is shown in box. egfp exons is shown in shading/black. Introns is shown in shading/gray. Alternative exon is shown in uppercase letter/shading. AC17-4 aptamer is shown in uppercase letter/bold. Anti-5′ss is shown in lowercase letter/bold. bla gene (Amp^{R}) is shown in gray. The plasmid map based on Benchling is available from the link below: https://benchling.com/s/seq-kxeLdS2ELr9C4pnrZ8lr?m=slm-M8eUKmXbeUX SKPJpKyQe

FIG. 15 Aptamer sequence and predicted structure of circularly-permuted AC17-4 (cpAC17-4).

FIG. 16 SPR sensorgram of cpAC17-4-immobilized chip injected with ASP2905 solution. cpAC17-4 binds ASP2905 with a K_{D} of 30 nM.

FIG. 17 EGFP expression regulated by the exon-skipping riboswitches. AC17-4 aptamer in the exon-skipping riboswitches (ex169-AC17-4-a7, a8, a9) was replaced with cpAC17-4. cpAC17-4 functions as a part of riboswitch in HEK293 cells.

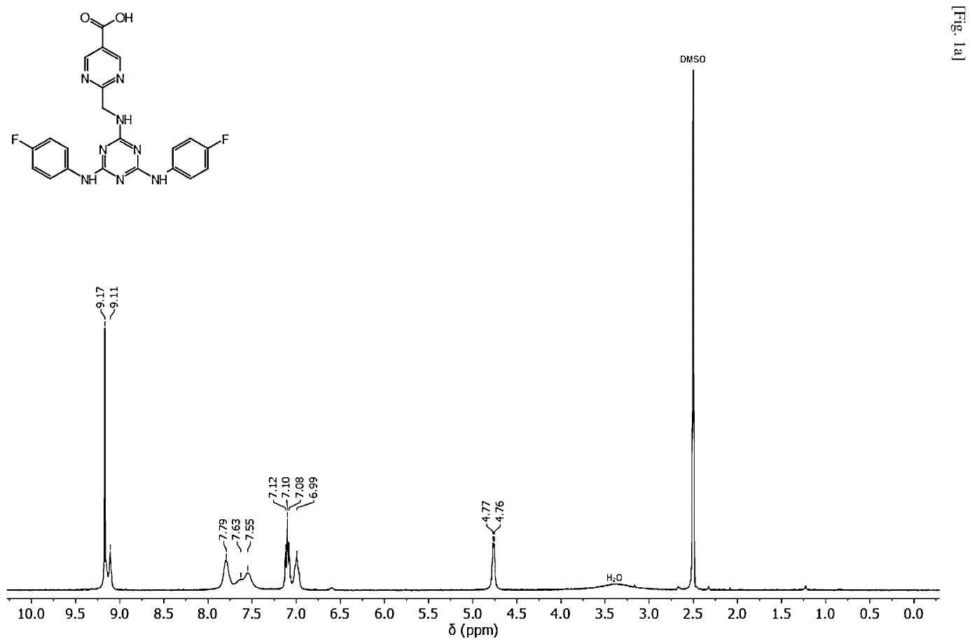

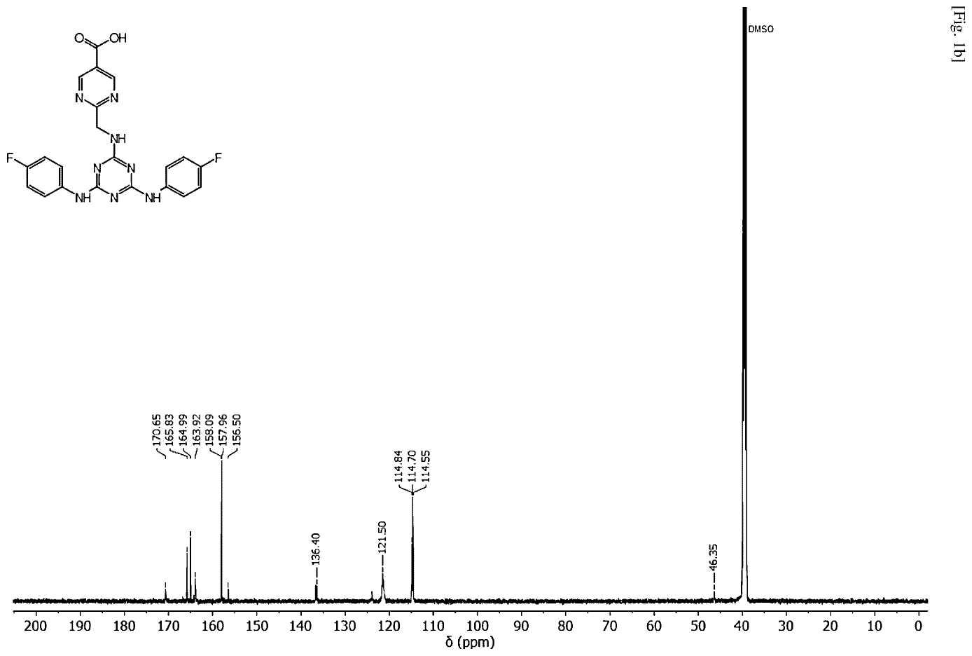

### DESCRIPTION OF THE EMBODIMENTS

To solve the above-mentioned problem, we have conducted in vitro selection of RNA aptamers against a small molecule ASP7967 whose structure is closely related to ASP2905, a known inhibitor of potassium voltage-gated channel sub-family H member 3 (KCNH3). One of the aptamers selected (AC17-4) was found to be functional in HEK293 cells, and it was used to design aptazyme-based riboswitches that can activate gene expression (>10-fold) in the presence of ASP2905 or ASP7967 at as low as 5 μM in the culture medium. An aptayzme-based riboswitch was successfully used to regulate human erythropoietin (hEPO) expression in mice injected with an adenoassociated virus (AAV8) vector using orally administered ASP7967. Furthermore, by combining aptazyme-based and exon-skipping riboswitch mechanisms, an ON/OFF ratio approaching 300 was achieved with a low basal expression level in cultured cells.

In the specification, the term “aptamer” refers to an oligonucleotide or peptide molecule that has high specificity and affinity for a specific substance. The term “aptamer” includes DNA aptamer, RNA aptamer, XNA aptamer and peptide aptamer. Aptamers can be of any length, e.g., from about 1 nucleotide to about 100 nucleotides, from about 5 nucleotides to about 50 nucleotides, or from about 10 nucleotides to about 25 nucleotides. An aptamer consisting of RNA is referred to as “RNA aptamer.”

In some embodiments of the present invention, the aptamer can be used in the form of a split aptamer. That is, the scope of the aptamer of the present invention encompasses split aptamers designed based on the parent aptamer. A split aptamer is an aptamer consisting of two fragments derived from the parent aptamer, obtained by cleaving the parent aptamer sequence, for example, at a loop sequence, which can bind the target substance by forming substantially the same structure as the parent aptamer when used. As readily understood by those skilled in the art, the split aptamers can be used for riboswitches and other applications, like the parent aptamer.

In other embodiments of the present invention, the RNA aptamer of the present invention is a circularly permutated aptamer. The term “circular permutation of an aptamer” or “circularly permutated aptamer” refers to an aptamer which has a changed order of nucleic acids in its nucleic acid sequence, as compared to the parent RNA sequence, that results in an RNA structure with different connectivity, but overall similar three-dimensional (3D) shape. A circular permutation of an aptamer is analogous to the mathematical notion of a cyclic permutation, in the sense that the sequence of the first portion of the parent aptamer (adjacent to the 5′-terminus) is related to the sequence of the second portion of the resulting circularly permutated aptamer (near its 3′-terminus). A circular permutation of an aptamer as compared to its parent aptamer is obtained through genetic or artificial engineering of the RNA sequence, whereby the 5′- and 3′-terminus of the parent RNA are “connected,” and the RNA sequence is interrupted at another site, to create a novel 5′- and 3′-terminus of said aptamer. Optionally, an additional sequence may be inserted between the original 5′- and 3′-termini. This additional sequence may form a stem loop structure. Alternatively, in cases where the region close to the original 5′- and 3′-termini form a stem structure, the length of the stem structure may be altered. The circularly permutated aptamers of the invention are the result of a connected 5′- and 3′-terminus of the parent aptamer sequence, and a cleavage or interrupted sequence at an accessible or exposed site (preferentially at a loop) of said aptamer, whereby the folding of the circularly permutated aptamer is retained or similar as compared to the folding of the parent aptamer. Said connection of the 5′- and 3′-terminus in said circularly permutated aptamer may be the result of a phosphodiester bond linkage, or of introducing an RNA linker, or of a deletion of a RNA sequence stretch near the original 5′- and 3′-terminus of the parent aptamer, followed by a phosphodiester bond between the remaining nucleic acids.

In the specification, the term “stem loop” represents a secondary structure of nucleotides with a “loop” consisting of unpaired nucleic acids and a “stem” formed by base pairs. A “stem” can be formed when the sequences of two regions of the same nucleotide strand are at least partially complementary, when they are substantially complementary, when they form wobble base pairs, etc. A “loop” represents a region of unpaired (i.e., non-complementary) nucleotides that connect the respective nucleotide strands of a stem and can cap the stem.

In the specification, the term “ribozyme” refers to a catalytic nucleic acid molecule that is RNA and that specifically recognizes and cleaves target nucleic acid sequences. The target may be the ribozyme itself or another nucleic acid molecule.

In the specification, the phrase “operably linked to” refers to the linking of multiple nucleic acids into a single nucleic acid such that the function of one nucleic acid is acted upon by another.

In the specification, regarding the descriptions of the embodiments, reference can be made to each other unless otherwise stated.

### <Aptamer>

The RNA aptamer of the present invention is, as mentioned above, an RNA aptamer that binds to ASP7967 or an analogue thereof, comprising a sequence:

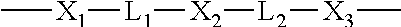

wherein
   X₁ has the sequence Y₁GY₂GY₃Y₄Y₅,
   L is a first stem loop nucleotide sequence comprising a first stem region, a first loop region and a second stem region (in this order), wherein the first stem region and the second stem region are 2 or more base pair long and are substantially complementary to each other;
   X₂ is A, G, C or U,
   L₂ is a second stem loop nucleotide sequence comprising a third stem region, a second loop region and a fourth stem region (in this order), wherein the third stem region and the fourth stem region are 2 or more base pair long and are substantially complementary to each other; and wherein the first base (5′-most base) in the third stem region is G and the last base (3′-most base) in the fourth stem region is C;
   X₃ has the sequence UY₆; and
   Y₁, Y₂, Y₃, Y₄, Y₅, and Y₆ are, each independently, A, G, C or U; or
a sequence:

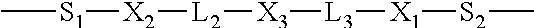

wherein
   S₁ and S₂ are, each independently, A, G, C or U, and S₁ and S₂ are capable of forming a base pair or a wobble base pair with each other;
   L₃ is a third stem loop nucleotide sequence comprising a fifth stem region, a third loop region and a sixth stem region, wherein the fifth stem region and the sixth stem region are 1 or more base pair long and are substantially complementary to each other; and
   X₁, X₂, X₃, and L₂ are as defined above; or
a sequence:

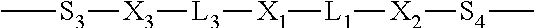

wherein
   S₃ is C and S₄ is G; and
   X₁, X₂, X₃, L₁, and L₃ are as defined above.

The above sequences are described from the left to the right, from 5′ end to 3′ end.

In some embodiments, the RNA aptamer of the present invention may be circularly permuted. In other embodiments, the present invention also provides a circularly permuted RNA aptamer derived from the RNA aptamer of the present invention. In the circularly permuted RNA aptamer, the length of the first, second and/or third stem loop region may be longer than in the unpermuted (parent) aptamer by 1, 2, 3, 4, or 5 base pairs, or may be shorter than in the unpermuted (parent) aptamer by 1, 2, 3, or 4 base pairs. The cleavage site may be in the first loop region, the second loop region or the third loop region.

In other embodiments, the RNA aptamer of the present invention may be a split aptamer.

In the present invention, Y₂ is preferably A or U, more preferably A. Y₃ is preferably A or U, more preferably A. Y₄ is preferably G or C, more preferably G.

In the present invention, preferably Y₁ and Y₆ are capable of forming a base pair or a wobble base pair with each other, more preferably Y₁ is G and Y₆ is U; or Y₁ is U and Y₆ is G.

In the present invention, preferably the first stem region and the second stem region are 3 to 7 base pair long and are substantially complementary to each other, more preferably the first stem region and the second stem region are 5 base pair long and are substantially complementary to each other especially preferably, the first stem region has the sequence GACGG and the second stem region has the sequence CCGUC.

In the present invention, preferably the first loop region has 3 to 7 bases, more preferably the first loop region has the sequence AUU or UUCG.

In the present invention, preferably the third stem region and the fourth stem region have 1 to 5 base pairs and are substantially complementary to each other, more preferably the third stem region and the fourth stem region have 3 or 4 base pairs and are substantially complementary to each other, especially preferably the third stem region has the sequence GCG and the fourth stem region has the sequence CGC; or the third stem region has the sequence GCGU and the fourth stem region has the sequence ACGC.

In the present invention, preferably the second loop region has 3 to 7 bases, more preferably the second loop region has the sequence AAUUCA or UUCG.

In the present invention, preferably the fifth stem region and the six stem region are 3 to 7 base pair long and are substantially complementary to each other, more preferably the fifth stem region and the six stem region are 4 base pair long and are substantially complementary to each other especially preferably, the fifth stem region has the sequence CUUG and the sixth stem region has the sequence CAAG.

In the present invention, preferably the third loop region has 3 to 7 bases, more preferably the third loop region has the sequence UUCG.

In the present invention, preferably S₁ is C and S₂ is G; or S₁ is G and S₂ is C.

The RNA aptamer of the present invention has the above sequence (-X₁-L₁-X₂-L₂-X₃-; -S₁-X₂-L₂-X₃-L₃-X₁-S₂-; -S₃-X₃-L₃-X₁-L₁-X₂-S₄-) and can bind to ASP7967 or an analogue thereof, preferably to ASP7967 or ASP2905. Here, ASP2905 is a compound represented by the following formula. ASP2905 is a potent and selective inhibitor of potassium channel Kv12.2 encoded by the Kcnh3/BEC1 gene. ASP2905 can cross the blood-brain barrier and has antipsychotic activities.

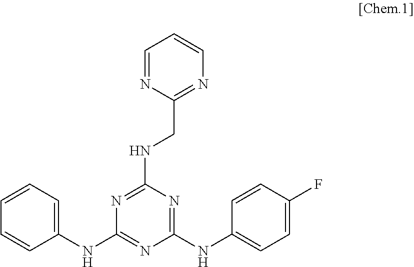 [SMILES: Fc1ccc(Nc2nc(NCc3ncccn3)nc(Nc3ccccc3)n2)cc1]

ASP7967 (ASP2905 analog) is a compound represented by the following formula.

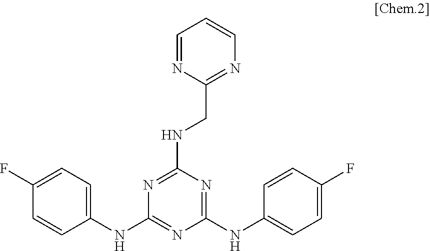 [SMILES: Fc1ccc(Nc2nc(NCc3ncccn3)nc(Nc3ccc(F)cc3)n2)cc1]

In the above sequence (-X₁-L₁-X₂-L₂-X₃-; -S₁-X₂-L₂-X₃-L₃-X₁-S₂-; -S₃-X₃-L₃-X₁-L₁-X₂-S₄-), the first stem region and the second stem region are 2 or more base pair long and are substantially complementary to each other; and the third stem region and the fourth stem region are 2 or more base pair long and are substantially complementary to each other; and the fifth stem region and the sixth stem region are 2 or more base pair long and are substantially complementary to each other. These complementary base pairs can form stem structures which lead to the ASP7967 aptamer-like structure with a binding activity to ASP7967 or an analogue thereof. While a stem structure can be formed by substantially complimentary base pairs (also including wobble base pairs (e.g., G=U base pairs) in addition to Watson-Crick base pairs), the number of base pairs for the above stem structure is not particularly limited. The number of base pairs is 2 or more base pair long, preferably 3 or more base pair long, more preferably 4 or more base pair long. The upper limit of the number of base pairs is not particularly limited, for example, it is 7 or less base pair long, preferably 6 or less base pair long, more preferably 5 or less base pair long.

In the present invention, the phrase “substantially complementary” includes not only cases that one sequence is completely complement to another sequence but also cases that there are one to several mismatches (including a bulge) which does not interfere with the stem structure formation. In the stem structure, even when base pairs are not formed in a part thereof, the above binding activity to ASP7967 or an analogue thereof is maintained as long as the aptamer structure is constituted as a whole. In the present invention, wobble base pair (e.g., G=U base pairs) is also included in “complementary base pair”.

In other aspects, the aptamer of the disclosure may include AC17-4 core sequence (SEQ ID NO: 1) as shown below.

(SEQ ID NO: 1)
5′-UGAGAGAGACGGAUUCCGUCCGCGAAUUCACGCUG-3′

In other aspects, the aptamer of the disclosure may include variants of SEQ ID NO: 1 as shown below.

(SEQ ID NO: 2)
5′-UGUGAGAGACGGAUUCCGUCCGCGAAUUCACGCUG-3′
(SEQ ID NO: 3)
5′-UGAGUGAGACGGAUUCCGUCCGCGAAUUCACGCUG-3′
(SEQ ID NO: 4)
5′-UGAGACAGACGGAUUCCGUCCGCGAAUUCACGCUG-3′
(SEQ ID NO: 5)
5′-UGAGAGUGACGGAUUCCGUCCGCGAAUUCACGCUG-3′
(SEQ ID NO: 6)
5′-UGAGAGAGACGGAUUCCGUCAGCGAAUUCACGCUG-3′
(SEQ ID NO: 7)
5′-GGAGAGAGACGGAUUCCGUCCGCGAAUUCACGCUU-3′
(SEQ ID NO: 8)
5′-UGAGAGACACGGAUUCCGUGCGCGAAUUCACGCUG-3′
(SEQ ID NO: 9)
5′-UGAGAGAGACGGUUCGCCGUCCGCGAAUUCACGCUG-3′
(SEQ ID NO: 10)
5′-UGAGAGAGACGGAUUCCGUCCGCGUUUCGACGCUG-3′
(SEQ ID NO: 11)
5′-UGAGAGAGACGGAUUCCGUCCGCGAAUUCGCGCUG-3′
(SEQ ID NO: 12)
5′-UGAGAGAGACGGAUUCCGUCCGCGUAUUCACGCUG-3′

In other aspects, the aptamer of the disclosure may include various aptamers designed based on AC17-4 aptamer (SEQ ID NO: 50) by any techniques well known in the art. For example, such an aptamer includes circularly-permuted AC17-4 (cpAC17-4) as shown below.

(SEQ ID NO: 77)
5′-GGUGUCCGCGAAUUCACGCUGCUUGUUCGCAAGUGAGAGAGA
CACC-3′

In other aspects, the aptamer of the disclosure may include cpAC17-4 core sequence (SEQ ID NO: 78) as shown below.

(SEQ ID NO: 78)
5′-CCGCGAAUUCACGCUGCUUGUUCGCAAGUGAGAGAG-3′

In other aspects, the aptamer of the disclosure may include the base sequence represented by any one of SEQ ID NOs: 1 to 12, 77 and 78; or the homologous base sequence having at least 60%, 70%, 80%, 90% or 95% identity to the base sequence represented by any one of SEQ ID NOs: 1 to 12, 77 and 78. The homologous base sequence may be the base sequence comprising substitution(s), deletion(s), and/or insertion(s) of 1 to 5 base(s) in the sequence represented by any one of SEQ ID NOs: 1 to 12, 77 and 78. The number of substitution(s), deletion(s), and insertion(s) of the base sequence is preferably 1 to 4 nucleotide(s), more preferably 1 to 3 nucleotide(s), more preferably 1 or 2 nucleotide(s).

In the present invention, the RNA aptamer having the sequence (-X₁-L₁-X₂-L₂-X₃-) may further comprise a fifth stem region adjacent to the 5′ terminus of X₁ and a sixth stem region adjacent to the 3′ terminus of X₃, wherein the fifth stem region and the sixth stem region have 1 to 15 base pairs and are substantially complementary to each other, and wherein the fifth stem region and the sixth stem region form a double-stranded stem. This double-stranded stem is added to stabilize the aptamer structure substantially formed by the above sequence (-X₁-L₁-X₂-L₂-X₃-). It is thought that the double-stranded stem has little effect on the binding force and binding specificity of the aptamer. Here, while the double-stranded stem structure can be formed by substantially complimentary base pairs (also including wobble base pairs (e.g., G=U base pairs) in addition to Watson-Crick base pairs), the number of base pairs is not particularly limited. The number of base pairs the fifth stem region and the sixth stem region have is preferably 2 to 10 base pairs, more preferably 6 to 9 base pairs, especially preferably 4 to 6 base pairs.

For example, the fifth stem region have the sequence as shown below.

5′-GCAAG-3′

For example, the sixth stem region have the sequence as shown below.

5′-CUUGC-3′

In the present invention, the RNA aptamer having the sequence (-S₁-X₂-L₂-X₃-L₃-X₁-S₂-) may further comprise a seventh stem region adjacent to the 5′ terminus of S₁ and a eighth stem region adjacent to the 3′ terminus of S₂, wherein the seventh stem region and the eighth stem region have 1 to 15 base pairs and are substantially complementary to each other, and wherein the seventh stem region and the eighth stem region form a double-stranded stem. This double-stranded stem is added to stabilize the aptamer structure substantially formed by the above sequence (-S₁-X₂-L₂-X₃-L₃-X₁-S₂-). The number of base pairs the seventh stem region and the eighth stem region have is preferably 2 to 10 base pairs, more preferably 6 to 9 base pairs, especially preferably 4 to 6 base pairs.

For example, the seventh stem region have the sequence as shown below.

5′-GGUGU-3′

For example, the eighth stem region have the sequence as shown below.

5′-ACACC-3′

In the present invention, the RNA aptamer having the sequence (-S₃-X₃-L₃-X₁-L₁-X₂-S₄-) may further comprise a ninth stem region adjacent to the 5′ terminus of S₃ and a tenth stem region adjacent to the 3′ terminus of S₄, wherein the ninth stem region and the tenth stem region have 1 to 15 base pairs and are substantially complementary to each other, and wherein the ninth stem region and the tenth stem region form a double-stranded stem. This double-stranded stem is added to stabilize the aptamer structure substantially formed by the above sequence (-S₃-X₃-L₃-X₁-L₁-X₂-S₄-). The number of base pairs the ninth stem region and the tenth stem region have is preferably 2 to 10 base pairs, more preferably 6 to 9 base pairs, especially preferably 4 to 6 base pairs.

The RNA aptamer of the present invention is, for example, a single-stranded RNA having a function of binding to ASP7967 or an analogue thereof. In the present specification, the base sequence is described from the left to the right, from 5′ end to 3′ end.

In the present invention, the binding force of the RNA aptamer to a substance is, for example, represented by a dissociation constant (Kd) of the RNA aptamer with the substance. The dissociation constant by SPR of the RNA of the present invention for ASP7967 is, for example, no more than 1.0 μM, preferably no more than 0.2 μM, and more preferably no more than 50 nM. The dissociation constant by SPR of the RNA of the present invention for ASP2905 is, for example, no more than 1.0 μM, preferably no more than 0.2 μM, and more preferably no more than 50 nM.

In the present invention, the RNA aptamer can specifically bind to ASP7967 or an analogue thereof. Here, the term “specific” or “specifically” in the present specification refers to a selective binding of an RNA aptamer of the present invention to ASP7967 or an analogue thereof. The binding specificity of an RNA aptamer can be examined by comparing the binding of the RNA aptamer to ASP7967 or an analogue thereof (the binding force to ASP7967 or an analogue thereof) to the binding of the RNA aptamer to an irrelevant substance (the binding force to an irrelevant substance), under a predetermined condition.

The RNA aptamer of the present invention may be linked to other polynucleotide.

### <Riboswitch>

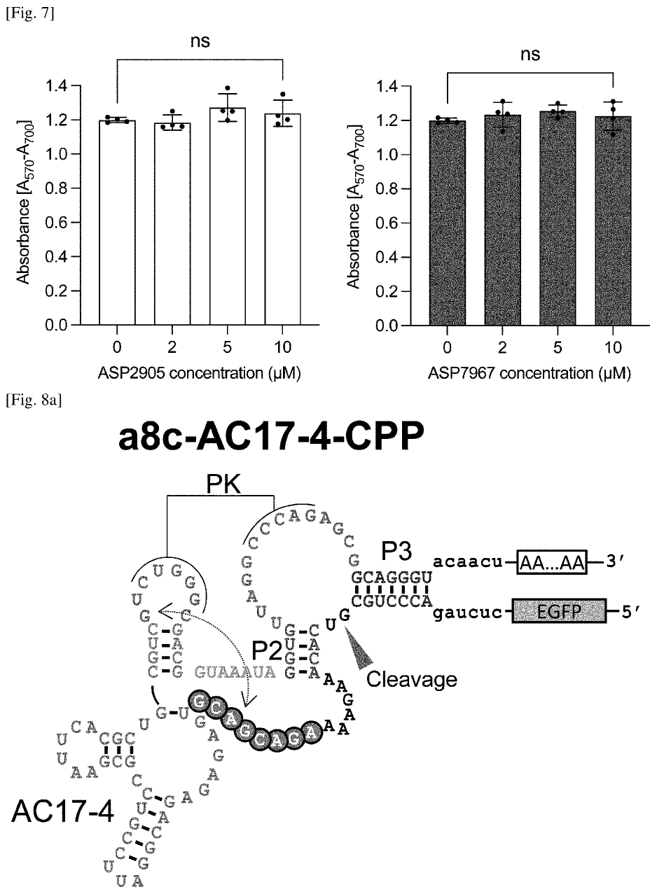

Riboswitch generally refers to a functional unit (region or segment) of an RNA polynucleotide for regulating activity of a target sequence such as sequence encoding a protein, non-protein coding RNA (such as siRNA, pre-miRNA), and the like on the same RNA polynucleotide. Riboswitch typically comprises an aptamer as a sensor region that detects the presence of a ligand such as a small molecule and an effector region that is involved in the basic function. Non-limiting basic functions of riboswitch include the formation of hairpin structures that terminate transcription, blocking translation by repressing ribosome binding sites, self-cleavage, and regulation of selective splicing. Riboswitch undergoes a structural change through the structural change of the aptamer caused by binding to the ligand, resulting in enhancing or preventing activity of a target sequence on the same RNA polynucleotide. Here, ASP7967 or its analog (ASP2095) are not cytotoxic (see FIG. 7), thus these molecules are excellent ligands for regulating the riboswitch function, especially in vivo.

The riboswitch of the present invention comprises the RNA aptamer of the present invention in a part thereof. The riboswitch of the present invention can detect ASP7967 or an analogue thereof through the aptamer and regulate activity of a target sequence on the same RNA polynucleotide.

In many embodiments, the riboswitch of the present invention may be operably linked to a target sequence such that the structural change of the aptamer in response to the binding to the ASP7967 or an analogue thereof results in enhancing or preventing activity of a target sequence. Further, the riboswitch of the present invention may be indirectly linked to a target sequence with any base sequences between them as long as the riboswitch can regulate the activity of the target sequence. Also, the riboswitch of the present invention may be positioned between a part of the target sequence and another part of the target sequences as long as the riboswitch can regulate the activity of the target sequence. In the present invention, the phrase “operably linked to” includes these cases. Further, in the present invention, a riboswitch sequence and a target sequence may share a part of sequence.

In the present invention, the target sequence may be a sequence encoding a protein or a non-protein coding RNA such as siRNA, pre-miRNA, pri-miRNA, sgRNA, lncRNA, RNA aptamer, ribozyme, tRNA, or rRNA. Here, the protein encoded by the target sequence may be any protein.

The target sequence may include and/or be operably linked to functional elements for allowing the target sequence to be transcribed and translated and/or expressed under appropriate conditions. The ordinarily skilled artisan appreciates that transcription, translation or expression control sequences can be appropriately selected based on their ability.

In the present invention, any riboswitch found in nature can be used as a platform for preparing the riboswitch of the present invention. Any riboswitch found in nature can be engineered to comprise the aptamer of the present invention instead of the original aptamer. Such a riboswitch may be further re-engineered as long as the riboswitch activity is maintained as a whole. Examples of the riboswitch found in nature as mentioned above include, but are not limited to, a thiamine pyrophosphate (TPP) riboswitch, an adenosine cobalamin (AdoCbl) riboswitch, an S-adenosyl methionine (SAM) riboswitch, an SAH riboswitch, a flavin mononucleotide (FMN) riboswitch, a tetrahydrofolate riboswitch, a lysine riboswitch, a glycine riboswitch, a purine riboswitch, a GlmS riboswitch, and a pre-queosine₁ (PreQ1) riboswitch.

### <Polynucleotide>

The polynucleotide of the present invention comprises a riboswitch comprising an RNA aptamer capable of binding to ASP7967 or an analogue thereof, or a DNA sequence that is capable of being transcribed into the riboswitch; and a target sequence encoding a protein, wherein the riboswitch is operably linked to the target sequence such that expression of the protein is upregulated or downregulated in response to ASP7967 or the analogue thereof.

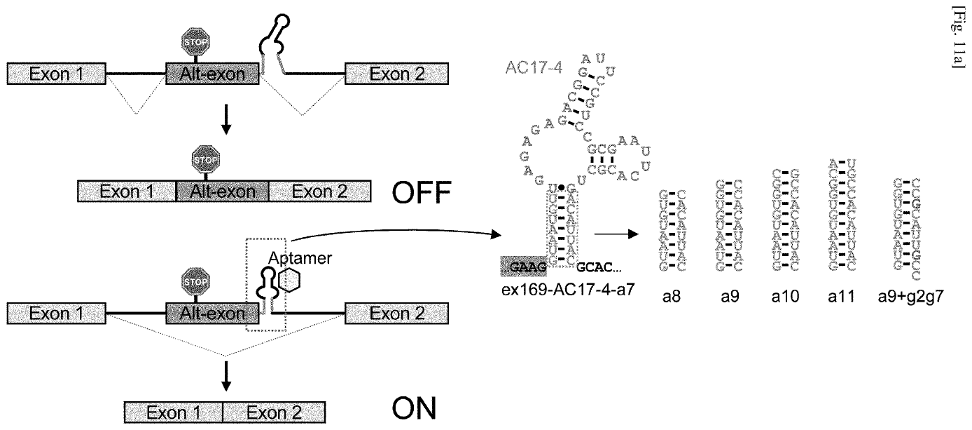

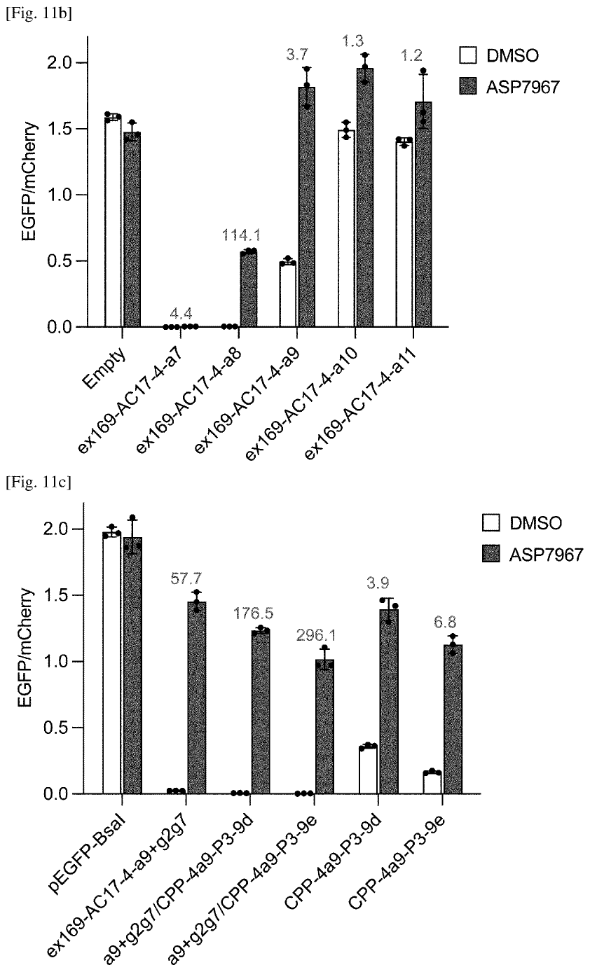

In some embodiment, the target sequence encoding a protein comprises a plurality of exons. The target sequence comprises, for example, an alternatively-spliced exon, flanked by a 5′ intron and a 3′ intron, wherein the alternatively-spliced exon comprises a stop codon that is in-frame with the protein when the alternatively-spliced exon is spliced into an mRNA of the protein. In this embodiment, for example, the riboswitch is positioned within the 3′ intron of the alternatively-spliced exon. The riboswitch comprises the 5′ splice site (“5′ ss”) sequence of the 3′ intron (i.e., the intronic splice site sequence that is immediately 3′ of the alternative exon) and sequence complimentary to the 5′ ss sequence of the 3′ intron as a effector region. When the aptamer binds the ligand, the effector region forms a stem and thus prevents splicing to the splice donor site at the 3′ end of the alternative exon, resulting in the expression of the protein of interest. Under certain conditions (for example, when the aptamer is not bound to its ligand), the effector region is in a context that provides access to the splice donor site at the 3′ end of the alternative exon leading to inclusion of the alternative exon in the mRNA of the protein, resulting in the inhibited expression of the protein of interest (see, e.g., FIG. 11a). In this case, the total length of the stem the effector region forms is preferably between 6 to 12 base pairs, more preferably between 6 to 10 base pairs, especially preferably between 7 to 9 base pairs.

In the embodiment, examples of the base sequence of the riboswitch of the present invention are shown below.

(SEQ ID NO: 13)
5′-GUAAUGUUGAGAGAGACGGAUUCCGUCCGCGAAUUCACGCUGA
CAUUAC-3′
(SEQ ID NO: 14)
5′- GUAAUGUGUGAGAGAGACGGAUUCCGUCCGCGAAUUCACGCU
GCACAUUAC-3′
(SEQ ID NO: 15)
5′-GUAAUGUGGUGAGAGAGACGGAUUCCGUCCGCGAAUUCACGCU
GCCACAUUAC-3′
(SEQ ID NO: 16)
5′-GUAAUGUGGCUGAGAGAGACGGAUUCCGUCCGCGAAUUCACGC
UGGCCACAUUAC-3′
(SEQ ID NO: 17)
5′-GUAAUGUGGCAUGAGAGAGACGGAUUCCGUCCGCGAAUUCACG
CUGUGCCACAUUAC-3′
(SEQ ID NO: 18)
5′-GUAAUGUGGUGAGAGAGACGGAUUCCGUCCGCGAAUUCACGCU
GCCGCAUUGCC-3′
(SEQ ID NO: 79)
5′-GUAAUGUCCGCGAAUUCACGCUGCUUGUUCGCAAGUGAGAGAG
ACAUUAC-3′
(SEQ ID NO: 80)
5′-GUAAUGUGCCGCGAAUUCACGCUGCUUGUUCGCAAGUGAGAGA
GCACAUUAC-3′
(SEQ ID NO: 81)
5′-GUAAUGUGGCCGCGAAUUCACGCUGCUUGUUCGCAAGUGAGAG
AGCCACAUUAC-3′

In some embodiment, the polynucleotide of the present invention comprises 3′ UTR comprising a polyadenylation signal sequence, and the riboswitch of the present invention is inserted within the 3′ UTR and at the 5′ side of the polyadenylation signal sequence. Here, the function of the polyadenylation signal sequence (including the function of the poly-(A) tail) is regulated by the riboswitch. In this embodiment, for example, the riboswitch comprises a self-cleaving ribozyme. Here a riboswitch sequence and a self-cleaving ribozyme may share a part of sequence.

In one embodiment, in the riboswitch, the self-cleaving ribozyme structure is stabilized by a stem structure formed by the terminal side sequences of the aptamer when the aptamer binds to ASP7967 or an analogue thereof (in this case, for example, the aptamer and the self-cleaving ribozyme share the stem structure), then the self-cleaving ribozyme is activated. The activated self-cleaving ribozyme cleaves itself inserted between the target sequence and the 3′ UTR, thereby the function of the polyadenylation signal sequence is inhibited.

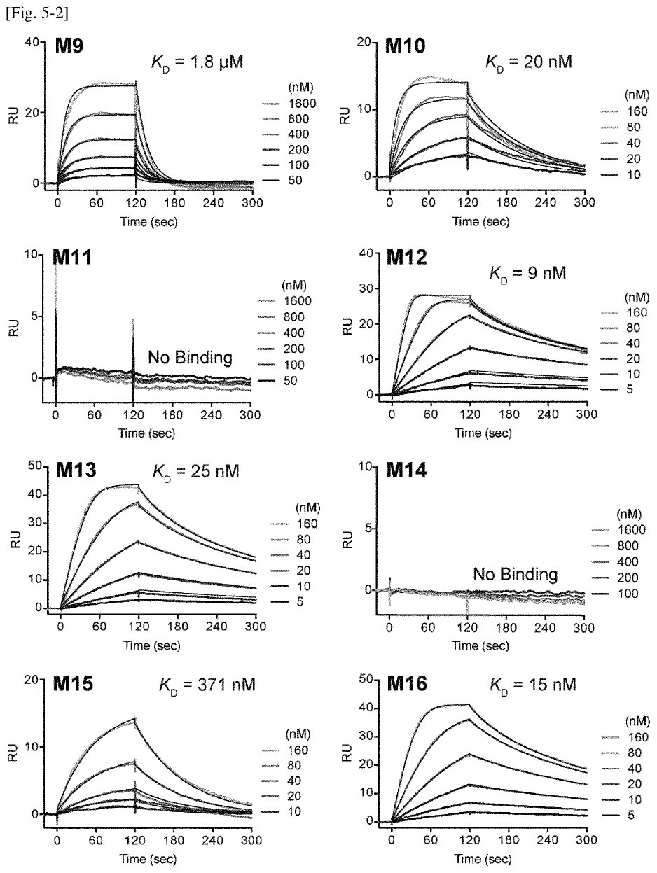

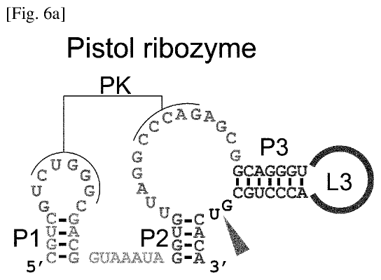

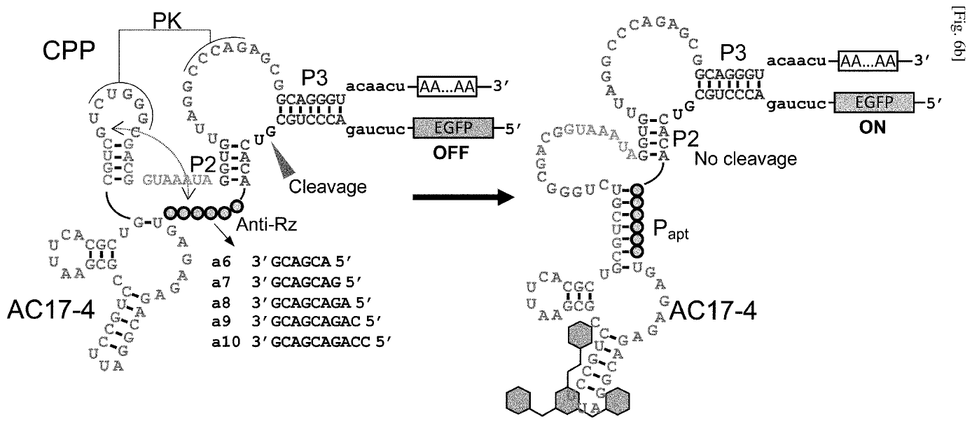

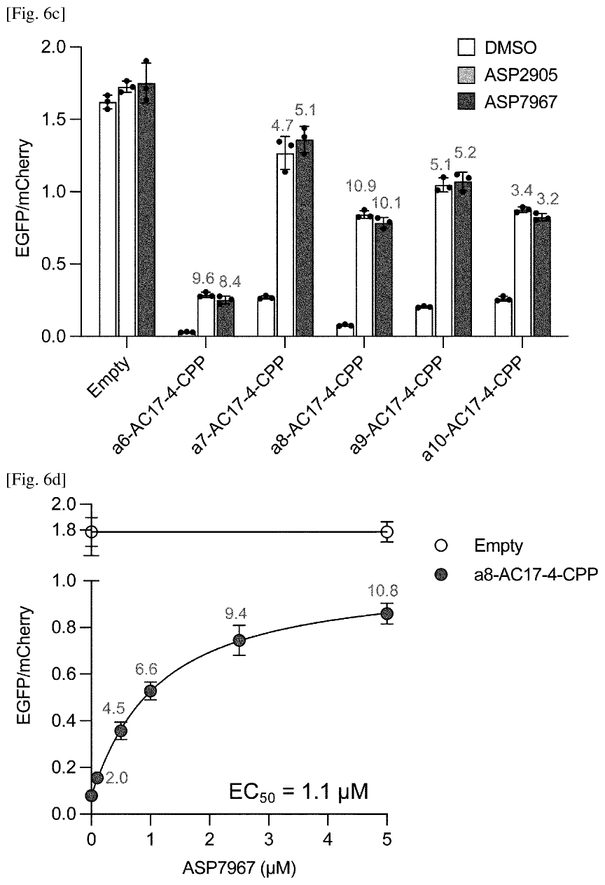

In one embodiment, in the riboswitch, the self-cleaving ribozyme structure is disrupted by a stem structure formed by the terminal side sequences of the aptamer when the aptamer binds to ASP7967 or an analogue thereof (in this case, for example, for the formation of the stem structure, the aptamer utilizes only one side sequence forming a stem of the self-cleaving ribozyme), then the self-cleaving ribozyme is inactivated (see, e.g., FIG. 6b). The inactivated self-cleaving ribozyme doesn't cleave itself inserted between the target sequence and the 3′ UTR, the function of the polyadenylation signal sequence is maintained. Here, the total length of the stem formed by the terminal side sequences of the aptamer as mentioned above is preferably between 6 to 11 base pairs, more preferably between 6 to 10 base pairs, especially preferably between 8 to 10 base pairs.

In the embodiment, examples of the base sequence of the riboswitch of the present invention are shown below.

(SEQ ID NO: 19)
5′-UCUAGACCCUGCGUCACAACGACGUGAGAGAGACGGAUUCCGU
CCGCGAAUUCACGCUGCGUCGUCUGGGCGACGGUAAAUAGGUGUUA
GGCCCAGAGCGGCAGGGUACAACUCCGGA-3′
(SEQ ID NO: 20)
5′-UCUAGACCCUGCGUCACAGACGACGUGAGAGAGACGGAUUCCG
UCCGCGAAUUCACGCUGCGUCGUCUGGGCGACGGUAAAUAGGUGUU
AGGCCCAGAGCGGCAGGGUACAACUCCGGA-3′
(SEQ ID NO: 21)
5′-UCUAGACCCUGCGUCACAAGACGACGUGAGAGAGACGGAUUCC
GUCCGCGAAUUCACGCUGCGUCGUCUGGGCGACGGUAAAUAGGUGU
UAGGCCCAGAGCGGCAGGGUACAACUCCGGA-3′
(SEQ ID NO: 22)
5′-UCUAGACCCUGCGUCACACAGACGACGUGAGAGAGACGGAUUC
CGUCCGCGAAUUCACGCUGCGUCGUCUGGGCGACGGUAAAUAGGUG
UUAGGCCCAGAGCGGCAGGGUACAACUCCGGA-3′
(SEQ ID NO: 23)
5′-UCUAGACCCUGCGUCACACCAGACGACGUGAGAGAGACGGAUU
CCGUCCGCGAAUUCACGCUGCGUCGUCUGGGCGACGGUAAAUAGGU
GUUAGGCCCAGAGCGGCAGGGUACAACU-3′
(SEQ ID NO: 24)
5′-UCUAGACCCUGCGUCACACCCAGACGACGUGAGAGAGACGGAU
UCCGUCCGCGAAUUCACGCUGCGUCGUCUGGGCGACGGUAAAUAGG
UGUUAGGCCCAGAGCGGCAGGGUACAACUCCGGA-3′
(SEQ ID NO: 25)
5′-UCUAGACCCUGCGUCACAAAGAAAAGACGACGUGAGAGAGACG
GAUUCCGUCCGCGAAUUCACGCUGCGUCGUCUGGGCGACGGUAAAU
AGGUGUUAGGCCCAGAGCGGCAGGGUACAACUCCGGA-3′
(SEQ ID NO: 26)
5′-UCUAGACCCUGCGUCACAUGAGAGAGACGGAUUCCGUCCGCGA
AUUCACGCUGUGUGACGCACGUCGUCUGGGCGACGGUAAAUAGGUG
UUAGGCCCAGAGCGGCAAGGUCUAACUCCGGA-3′
(SEQ ID NO: 27)
5′-UCUAGACCCUGCGUCACAUGAGAGAGACGGAUUCCGUCCGCGA
AUUCACGCUGUGUGACGCACGUCGUCUGGGCGACGGUAAAUAGGUG
UUAGGCCCAGAGCGGCAGAGUCUAACUCCGGA-3′

In the present invention, the protein encoded by the target sequence may be any protein, for example, may be a protein to be used for a therapy. The examples of the protein encoded by the target sequence include 4-1BB ligand, 5-helix, human C-C chemokine, human L105 chemokine, human L105 chemokine designated huL105_3., monokine induced by gamma-interferon (MIG), partial CXCR4B protein, platelet basic protein (PBP), α1-antitrypsin, ACRP-30 Homologue; Complement Component Clq C, Adenoid-expressed chemokine (ADEC), aFGF; FGF-1, AGF, AGF Protein, albumin, an etoposide, angiostatin, Anthrax vaccine, Antibodies specific for collapsin, antistasin, Anti-TGF beta family antibodies, antithrombin III, APM-1; ACRP-30; Famoxin, apo-lipoprotein species, Arylsulfatase B, b57 Protein, BCMA, Beta-thromboglobulin protein (beta-TG), bFGF; FGF2, Blood coagulation factors, BMP Processing Enzyme Furin, BMP-10, BMP-12, BMP-15, BMP-17, BMP-18, BMP-2B, BMP-4, BMP-5, BMP-6, BMP-9, Bone Morphogenic Protein-2, calcitonin, Calpain-10a, Calpain-10b, Calpain-10c, Cancer Vaccine, Carboxypeptidase, C-C chemokine, MCP2, CCR5 variant, CCR7, CCR7, CD11a Mab, CD137; 4-1BB Receptor Protein, CD20 Mab, CD27, CD27L, CD30, CD30 ligand, CD33 immunotoxin, CD40, CD40L, CD52 Mab, Cerebus Protein, Chemokine Eotaxin., Chemokine hIL-8, Chemokine hMCP1, Chemokine hMCP1a, Chemokine hMCP1b, Chemokine hMCP2, Chemokine hMCP3, Chemokine hSDF1b, Chemokine MCP-4, chemokine TECK and TECK variant, Chemokine-like protein IL-8M1 Full-Length and Mature, Chemokine-like protein IL-8M10 Full-Length and Mature, Chemokine-like protein IL-8M3, Chemokine-like protein IL-8M8 Full-Length and Mature, Chemokine-like protein IL-8M9 Full-Length and Mature, Chemokine-like protein PF4-414 Full-Length and Mature, Chemokine-like protein PF4-426 Full-Length and Mature, Chemokine-like protein PF4-M2 Full-Length and Mature, Cholera vaccine, Chondromodulin-like protein, c-kit ligand; SCF; Mast cell growth factor; MGF; Fibrosarcoma-derived stem cell factor, CNTF and fragment thereof, coagulation factors in both pre and active forms, collagens, Complement C5 Mab, Connective tissue activating protein-III, CTAA16.88 Mab, CTAP-III, CTLA4-Ig, CTLA-8, CXC3, CXC3, CXCR3; CXC chemokine receptor 3, cyanovirin-N, Darbepoetin, designated exodus, designated huL105_7., DIL-40, Dnase, EDAR, EGF Receptor Mab, ENA-78, Endostatin, Eotaxin, Epithelial neutrophil activating protein-78, EPO receptor; EPOR, erythropoietin (EPO) and EPO mimics, Eutropin, Exodus protein, Factor IX, Factor VII, Factor VIII, Factor X and Factor XIII, FAS Ligand Inhibitory Protein (DcR3), FasL, FasL, FasL, FGF, FGF-12; Fibroblast growth factor homologous factor-1, FGF-15, FGF-16, FGF-18, FGF-3; INT-2, FGF-4; gelonin, HST-1; HBGF-4, FGF-5, FGF-6; Heparin binding secreted transforming factor-2, FGF-8, FGF-9; Glia activating factor, fibrinogen, flt-1, flt-3 ligand, Follicle stimulating hormone Alpha subunit, Follicle stimulating hormone Beta subunit, Follitropin, Fractalkine, fragment. myofibrillar protein Troponin I, FSH, Galactosidase, Galectin-4, G-CSF, GDF-1, Gene therapy, Glioma-derived growth factor, glucagon, glucagon-like peptides, Glucocerebrosidase, glucose oxidase, Glucosidase, Glycodelin-A; Progesterone-associated endometrial protein, GM-CSF, gonadotropin, Granulocyte chemotactic protein-2 (GCP-2), Granulocyte-macrophage colony stimulating factor, growth hormone, Growth related oncogene-alpha (GRO-alpha), Growth related oncogene-beta (GRO-beta), Growth related oncogene-gamma (GRO-gamma), hAPO-4; TROY, hCG, Hepatitus B surface Antigen, Hepatitus B Vaccine, HER2 Receptor Mab, hirudin, HIV gp120, HIV gp41, HIV Inhibitor Peptide, HIV Inhibitor Peptide, HIV Inhibitor Peptide, HIV protease inhibiting peptides, HIV-1 protease inhibitors, HPV vaccine, Human 6CKine protein, Human Act-2 protein, Human adipogenesis inhibitory factor, human B cell stimulating factor-2 receptor, Human beta-chemokine H1305 (MCP-2), Human C-C chemokine DGWCC, Human CC chemokine ELC protein, Human CC type chemokine interleukin C, Human CCC3 protein, Human CCF18 chemokine, Human CC-type chemokine protein designated SLC (secondary lymphoid chemokine), Human chemokine beta-8 short forms, Human chemokine C10, Human chemokine CC-2, Human chemokine CC-3, Human chemokine CCR-2, Human chemokine Ckbeta-7, Human chemokine ENA-78, Human chemokine eotaxin, Human chemokine GRO alpha, Human chemokine GROalpha, Human chemokine GRObeta, Human chemokine HCC-1, Human chemokine HCC-1, Human chemokine 1-309, Human chemokine IP-10, Human chemokine L105_3, Human chemokine L105_7, Human chemokine MIG, Human chemokine MIG-beta protein, Human chemokine MIP-1alpha, Human chemokine MIP1beta, Human chemokine MIP-3alpha, Human chemokine MIP-3beta, Human chemokine PF4, Human chemokine protein 331D5, Human chemokine protein 61164, Human chemokine receptor CXCR3, Human chemokine SDF1alpha, Human chemokine SDF1beta, Human chemokine ZSIG-35, Human Chr19Kine protein, Human CKbeta-9, Human CKbeta-9, Human CX3C 111 amino acid chemokine, Human DNAX interleukin-40, Human DVic-1 C-C chemokine, Human EDIRF I protein sequence, Human EDIRF II protein sequence, Human eosinocyte CC type chemokine eotaxin, Human eosinophil-expressed chemokine (EEC), Human fast twitch skeletal muscle troponin C, Human fast twitch skeletal muscle troponin I, Human fast twitch skeletal muscle Troponin subunit C, Human fast twitch skeletal muscle Troponin subunit I Protein, Human fast twitch skeletal muscle Troponin subunit T, Human fast twitch skeletal muscle troponin T, Human foetal spleen expressed chemokine, FSEC, Human GM-CSF receptor, Human gro-alpha chemokine, Human gro-beta chemokine, Human gro-gamma chemokine, Human IL-16 protein, Human IL-1RD10 protein sequence, Human IL-1RD9, Human IL-5 receptor alpha chain, Human IL-6 receptor, Human IL-8 receptor protein hIL8RA, Human IL-8 receptor protein hIL8RB, Human IL-9 receptor protein, Human IL-9 receptor protein variant #3, Human IL-9 receptor protein variant fragment, Human IL-9 receptor protein variant fragment #3, Human interleukin 1 delta, Human Interleukin 10, Human Interleukin 10, Human interleukin 18, Human interleukin 18 derivatives, Human interleukin-1 beta precursor, Human interleukin-1 beta precursor, Human interleukin-1 receptor accessory protein, Human interleukin-1 receptor antagonist beta, Human interleukin-1 type-3 receptor, Human Interleukin-10 (precursor), Human Interleukin-10 (precursor), Human interleukin-11 receptor, Human interleukin-12 40 kD subunit, Human interleukin-12 beta-1 receptor, Human interleukin-12 beta-2 receptor, Human Interleukin-12 p35 protein, Human Interleukin-12 p40 protein, Human interleukin-12 receptor, Human interleukin-13 alpha receptor, Human interleukin-13 beta receptor, Human interleukin-15, Human interleukin-15 receptor from clone P1, Human interleukin-17 receptor, Human interleukin-18 protein (IL-18), Human interleukin-3, human interleukin-3 receptor, Human interleukin-3 variant, Human interleukin-4 receptor, Human interleukin-5, Human interleukin-6, Human interleukin-7, Human interleukin-7, Human interleukin-8 (IL-8), Human intracellular IL-1 receptor antagonist, Human IP-10 and HIV-1 gp120 hypervariable region fusion protein, Human IP-10 and human Muc-1 core epitope (VNT) fusion protein, human liver and activation regulated chemokine (LARC), Human Lkn-1 Full-Length and Mature protein, Human mammary associated chemokine (MACK) protein Full-Length and Mature, Human mature chemokine Ckbeta-7, Human mature gro-alpha, Human mature gro-gamma polypeptide used to treat sepsis, Human MCP-3 and human Muc-1 core epitope (VNT) fusion protein, Human MI10 protein, Human MILA protein, Human monocyte chemoattractant factor hMCP-1, Human monocyte chemoattractant factor hMCP-3, Human monocyte chemotactic proprotein (MCPP) sequence, Human neurotactin chemokine like domain, Human non-ELR CXC chemokine H174, Human non-ELR CXC chemokine IP10, Human non-ELR CXC chemokine Mig, Human PAI-1 mutants, Human protein with IL-16 activity, Human protein with IL-16 activity, Human secondary lymphoid chemokine (SLC), Human SISD protein, Human STCP-1, Human stromal cell-derived chemokine, SDF-1, Human T cell mixed lymphocyte reaction expressed chemokine (TMEC), Human thymus and activation regulated cytokine (TARC), Human thymus expressed, Human TNF-alpha, Human TNF-alpha, Human TNF-beta (LT-alpha), Human type CC chemokine eotaxin 3 protein sequence, Human type II interleukin-1 receptor, Human wild-type interleukin-4 (hIL-4) protein, Human ZCHEMO-8 protein, Humanized Anti-VEGF Antibodies, and fragments thereof, Humanized Anti-VEGF Antibodies, and fragments thereof, Hyaluronidase, ICE 10 kD subunit, ICE 20 kD subunit, ICE 22 kD subunit, Iduronate-2-sulfatase, Iduronidase, IL-1 alpha, IL-1 beta, IL-1 inhibitor (IL-1i), IL-1 mature, IL-10 receptor, IL-11, IL-11, IL-12 p40 subunit, IL-13, IL-14, IL-15, IL-15 receptor, IL-17, IL-17 receptor, II-17 receptor, II-17 receptor, IL-19, IL-1i fragments, IL1-receptor antagonist, IL-21 (TIF), IL-3 containing fusion protein, IL-3 mutant proteins, IL-3 variants, IL-3 variants, IL-4, IL-4 mutein, IL-4 mutein Y124G, IL-4 mutein Y124X, IL-4 muteins, II-5 receptor, IL-6, II-6 receptor, IL-7 receptor clone, IL-8 receptor, IL-9 mature protein variant (Met117 version), immunoglobulins or immunoglobulin-based molecules or fragment of either (e.g. a Small Modular ImmunoPharmaceutical™ (“SMIP”) or dAb, Fab′ fragments, F(ab′) 2, scAb, scFv or scFv fragment), including but not limited to plasminogen, Influenza Vaccine, Inhibin alpha, Inhibin beta, insulin, insulin-like growth factor, Integrin Mab, inter-alpha trypsin inhibitor, inter-alpha trypsin inhibitor, Interferon gamma-inducible protein (IP-10), interferons (such as interferon alpha species and sub-species, interferon beta species and sub-species, interferon gamma species and sub-species), interferons (such as interferon alpha species and sub-species, interferon beta species and sub-species, interferon gamma species and sub-species), Interleukin 6, Interleukin 8 (IL-8) receptor, Interleukin 8 receptor B, Interleukin-1alpha, Interleukin-2 receptor associated protein p43, interleukin-3, interleukin-4 muteins, Interleukin-8 (IL-8) protein, interleukin-9, Interleukin-9 (IL-9) mature protein (Thr117 version), interleukins (such as IL0, IL11 and IL2), interleukins (such as IL0, IL11 and IL2), Japanese encephalitis vaccine, Kalikrein Inhibitor, Keratinocyte growth factor, Kunitz domain protein (such as aprotinin, amyloid precursor protein and those described in WO 03/066824, with or without albumin fusions), Kunitz domain protein, protinin, amyloid precursor protein with or without albumin fusions, LACI, lactoferrin, Latent TGF-beta binding protein II, leptin, Liver expressed chemokine-1 (LVEC-1), Liver expressed chemokine-2 (LVEC-2), LT-alpha, LT-beta, Luteinization Hormone, Lyme Vaccine, Lymphotactin, Macrophage derived chemokine analogue MDC (n+1), Macrophage derived chemokine analogue MDC-eyfy, Macrophage derived chemokine analogue MDC-yl, Macrophage derived chemokine, MDC, Macrophage-derived chemokine (MDC), Maspin; Protease Inhibitor 5, MCP-1 receptor, MCP-1a, MCP-1b, MCP-3, MCP-4 receptor, M-CSF, Melanoma inhibiting protein, Membrane-bound proteins, Met117 human interleukin 9, MIP-3 alpha, MIP-3 beta, MIP-Gamma, MIRAP, Modified Rantes, monoclonal antibody, MP52, Mutant Interleukin 6 S₁₇₆R, myofibrillar contractile protein Troponin I, Natriuretic Peptide, Nerve Growth Factor-beta, Nerve Growth Factor-beta2, Neuropilin-1, Neuropilin-2, Neurotactin, Neurotrophin-3, Neurotrophin-4, Neurotrophin-4a, Neurotrophin-4b, Neurotrophin-4c, Neurotrophin-4d, Neutrophil activating peptide-2 (NAP-2), NOGO-66 Receptor, NOGO-A, NOGO-B, NOGO-C, Novel beta-chemokine designated PTEC, N-terminal modified chemokine GroHEK/hSDF-1alpha, N-terminal modified chemokine GroHEK/hSDF-1beta, N-terminal modified chemokine met-hSDF-1 alpha, N-terminal modified chemokine met-hSDF-1 beta, OPGL, Osteogenic Protein-1; OP-1; BMP-7, Osteogenic Protein-2, OX40; ACT-4, OX40L, Oxytocin (Neurophysin I), parathyroid hormone, Patched, Patched-2, PDGF-D, Pertussis toxoid, Pituitary expressed chemokine (PGEC), Placental Growth Factor, Placental Growth Factor-2, Plasminogen Activator Inhibitor-1; PAI-1, Plasminogen Activator Inhibitor-2; PAI-2, Plasminogen Activator Inhibitor-2; PAI-2, Platelet derived growth factor, Platelet derived growth factor Bv-sis, Platelet derived growth factor precursor A, Platelet derived growth factor precursor B, Platelet Mab, platelet-derived endothelial cell growth factor (PD-ECGF), Platelet-Derived Growth Factor A chain, Platelet-Derived Growth Factor B chain, polypeptide used to treat sepsis, Preproapolipoprotein “milano” variant, Preproapolipoprotein “paris” variant, pre-thrombin, Primate CC chemokine “ILINCK”, Primate CXC chemokine “IBICK”, proinsulin, Prolactin, Prolactin2, prosaptide, Protease inhibitor peptides, Protein C, Protein S, pro-thrombin, prourokinase, RANTES, RANTES 8-68, RANTES 9-68, RANTES peptide, RANTES receptor, Recombinant interleukin-16, Resistin, restrictocin, Retroviral protease inhibitors, ricin, Rotavirus Vaccine, RSV Mab, saporin, sarcin, Secreted and Transmembrane polypeptides, Secreted and Transmembrane polypeptides, serum cholinesterase, serum protein, blood clotting factor, Soluble BMP Receptor Kinase Protein-3, Soluble VEGF Receptor, Stem Cell Inhibitory Factor, Straphylococcus Vaccine, Stromal Derived Factor-1 alpha, Stromal Derived Factor-1 beta, Substance P (tachykinin), T1249 peptide, T20 peptide, T4 Endonuclease, TACI, Tarc, TGF-beta 1, TGF-beta 2, Thr117 human interleukin 9, thrombin, thrombopoietin, Thrombopoietin derivative1, Thrombopoietin derivative2, Thrombopoietin derivative3, Thrombopoietin derivative4, Thrombopoietin derivative5, Thrombopoietin derivative6, Thrombopoietin derivative7, Thymus expressed chemokine (TECK), Thyroid stimulating Hormone, tick anticoagulant peptide, Tim-1 protein, TNF-alpha precursor, TNF-R, TNF-RII; TNF p75 Receptor; Death Receptor, tPA, transferrin, transforming growth factor beta, Troponin peptides, Truncated monocyte chemotactic protein 2 (6-76), Truncated monocyte chemotactic protein 2 (6-76), Truncated RANTES protein (3-68), tumour necrosis factor, Urate Oxidase, urokinase, Vasopressin (Neurophysin II), VEGF R-3; flt-4, VEGF Receptor; KDR; flk-1, VEGF-110, VEGF-121, VEGF-138, VEGF-145, VEGF-162, VEGF-165, VEGF-182, VEGF-189, VEGF-206, VEGF-D, VEGF-E; VEGF-X, von Willebrand's factor, Wild type monocyte chemotactic protein 2, Wild type monocyte chemotactic protein 2, ZTGF-beta 9, β (T870)-globin, SMN1, chimeric antigen receptors, RPE65, F8, HGF, LPL, p53, apoe2, Arylsulfatase A, NAGLU, SGSH, AADC, GAD, GDNF, NRTN, LCAT, GBA, FGF-1, FGF-2, ADA, CLN2, CLN6, CLN3, IDS, Huntingtin, TRAIL, dystrophin, GALGT2, accA, IDUA, GLB1, FS344, SGCA, DYSF, ABCD1, Gigaxonin and functional fragments thereof.

The polynucleotide of the present invention may be one wherein a sugar residue (e.g., ribose) of each nucleotide has been modified (a modification to RNA will be described here, but description is appropriately read as a description for a modification to DNA). As examples of the modification in a sugar residue, substitution of hydroxyl group at the 2′-position, the 3′-position and/or 4′-position of the sugar residue with another atom, and the like can be mentioned. As the kind of the modification, fluorination, alkoxylation (e.g., methoxylation, ethoxylation), O-arylation, S-alkylation (e.g., S-methylation, S-ethylation), S-arylation, and amination (e.g., —NH₂) can be mentioned. Such alterations in the sugar residue can be performed by a method known per se (see, for example, Sproat et al., (1991) Nucl. Acid. Res. 19, 733-738; Cotton et al., (1991) Nucl. Acid. Res. 19, 2629-2635; Hobbs et al., (1973) Biochemistry 12, 5138-5145).

The sugar residue may also be BNA: Bridged nucleic acid (LNA: Linked nucleic acid), wherein a crosslinking structure is formed at the 2′-position and the 4′-position. Such alteration of the sugar residue can also be performed by a method known per se (e.g., Tetrahedron Lett., 38, 8735-8738 (1997); Tetrahedron, 59, 5123-5128 (2003), Rahman S. M. A., Seki S., Obika S., Yoshikawa H., Miyashita K., Imanishi T., J. Am. Chem. Soc., 130, 4886-4896 (2008) and the like).

The polynucleotide of the present invention may also have a nucleic acid base (e.g., purine or pyrimidine) altered (e.g., chemical substitution). As examples of such alterations, pyrimidine alteration at 5-position, purine alteration at 6- and/or 8-position(s), alteration with an extracyclic amine, substitution with 4-thiouridine, and substitution with 5-bromo or 5-iodo-uracil can be mentioned.

In addition, the phosphate group contained in the polynucleotide of the present invention may be altered to confer resistance to nuclease and hydrolysis. For example, the P(O)O group as a phosphoric acid group may be substituted by P(O)S (thioate), P(S)S (dithioate), P(O)NR₂ (amidate), P(O)R, R(O)OR′, CO or CH₂ (formacetal) or 3′-amine (—NH—CH₂—CH₂—) [wherein each unit of R or R′ is independently H or a substituted or unsubstituted alkyl (e.g., methyl, ethyl)].

### <Vector>

The present invention is also related to an RNA or DNA vector comprising the RNA aptamer of the present invention or a DNA sequence that is capable of being transcribed into the RNA aptamer of the present invention. An RNA vector comprises, for example, the RNA aptamer of the present invention. A DNA comprises, for example, a DNA sequence that is capable of being transcribed into the RNA aptamer of the present invention.

The present invention is also related to an RNA or DNA vector comprising the riboswitch of the present invention or a DNA sequence that is capable of being transcribed into the riboswitch of the present invention. An RNA vector comprises, for example, the riboswitch of the present invention. A DNA vector comprises, for example, a DNA sequence that is capable of being transcribed into the riboswitch of the present invention.

The present invention is also related to an RNA or DNA vector comprising the riboswitch of the present invention or a DNA sequence that is capable of being transcribed into the riboswitch of the present invention and a target sequence operably liked to the riboswitch or the DNA sequence. An RNA vector comprises, for example, the riboswitch of the present invention and a target sequence operably liked to the riboswitch. A DNA vector comprises, for example, a DNA sequence that is capable of being transcribed into the riboswitch of the present invention and a target sequence operably liked to the DNA sequence.

The present invention is also related to an RNA or DNA vector comprising the polynucleotide of the present invention. An RNA vector comprises, for example, the polynucleotide comprising: a riboswitch comprising an RNA aptamer capable of binding to ASP7967 or an analogue thereof and a target sequence encoding a protein, wherein the riboswitch is operably linked to the target sequence such that expression of the protein is upregulated or downregulated in response to ASP7967 or the analogue thereof. A DNA vector comprises, for example, a DNA sequence that is capable of being transcribed into the polynucleotide comprising: a riboswitch comprising an RNA aptamer capable of binding to ASP7967 or an analogue thereof and a target sequence encoding a protein, wherein the riboswitch is operably linked to the target sequence such that expression of the protein is upregulated or downregulated in response to ASP7967 or the analogue thereof.

In the present invention, examples of vectors include, but are not limited to, plasmids, viral vectors, cosmids, artificial chromosomes, and phagemids. The vector may be one which is able to replicate in a host cell, and which may be further characterized by one or more endonuclease restriction sites at which the vector can be cut and into which a desired nucleic acid sequence may be inserted. The vectors may comprise one or more marker sequences suitable for use in the identification and/or selection of cells which have or have not been transformed or genomically modified with the vector.

In the present invention, the vector may further comprise additional nucleic acid elements including nucleic acid regions or segments that provide for the replication of the vector in a cell and expression of the aptamer, the riboswitch, or the polynucleotide of the present invention in that cell at appropriate levels. The ordinarily skilled artisan appreciates that expression control sequences (promoters, enhancers, and the like) are selected based on their ability to promote expression of them in the cell.

In the present invention, viral vectors can be used preferably. Examples of viral vectors include, but are not limited to, adenoviral (AV) vectors, adeno-associated virus (AAV) vectors, retroviral and lentiviral vectors, Herpes simplex type 1 (HSV1) vectors, and Vesicular stomatitis virus (VSV) vectors.

In the present invention, polynucleotide and vector can be introduced into a cell by a viral vector system or nonviral vector system. In the nonviral vector system, for example, cationic lipids, polymers, or both as carriers can be used. Conjugated polyL-lysine (PLL) polymer and polyethylenimine (PEI) polymer systems can also be used to deliver the polynucleotide or vector to cells. Other methods for delivering the polynucleotide or vector to cells includes hydrodynamic injection and electroporation and use of ultrasound. For a review of viral and non-viral delivery systems for gene delivery see Nayerossadat, N. et al. (Adv Biomed Res. 2012; 1:27; incorporated herein by reference).

### <Kit>

The kit for regulating expression of a protein of the present invention comprises ASP7967 or an analogue thereof, and the polynucleotide of the present invention or a vector comprising the polynucleotide of the present invention. The polynucleotide of the present invention can be used for regulating (upregulating or downregulating) the expression of the specific protein in response to ASP7967 or the analogue thereof, therefore, the kit of the present invention can be used for regulating the expression of the specific protein which is encoded by the target sequence on the polynucleotide of the present invention.

As mentioned above, ASP2905 (ASP7967 analogue) is a potent and selective inhibitor of potassium channel Kv12.2 encoded by the Kcnh3/BEC1 gene, and can cross the blood-brain barrier and has antipsychotic activities. Therefore, the kit of the present invention may be used for treating, for example, the diseases, preferably a central nervous system disease, a cognitive disorder, or a KCNH3-related disease, more preferably ADHD, Parkinson's disease, Alzheimer's disease, or schizophrenia.

In the present invention, the protein encoded by the target sequence may be a protein to be used for a therapy. In this case, the kit of the present invention can be used for treating any diseases which the protein encoded by the target sequence would a therapeutic effect on. Examples of such a protein encoded by the target sequence are mentioned above in the section of the polynucleotide of the present invention.

The kit of the present invention may further comprise any components other than ASP7967 or an analogue thereof, and the polynucleotide of the present invention and a vector comprising the polynucleotide of the present invention, if necessary.

### <Method for Regulating Expression of a Protein in Vivo>

The method for regulating expression of a protein in vivo of the present invention comprises (1) introducing the polynucleotide of the present invention or a vector comprising the polynucleotide of the present invention into a cell, and (2) bringing ASP7967 or an analogue thereof into contact with the polynucleotide or the vector. The present method can regulate the expression of the protein encoded by the target sequence on the polynucleotide of the present invention in a cell such that the expression of the protein are upregulated or downregulated only when the polynucleotide or the vector is contacted with ASP7967 or an analogue thereof.

### <Method for Treating or Preventing a Disease>

The method for treating or preventing a disease of the present invention comprises (1) introducing the polynucleotide of the present invention or a vector comprising the polynucleotide of the present invention into a subject, and (2) administering ASP7967 or an analogue thereof into the subject. The present method can treat or prevent a disease due to the properties of ASP7967 or an analogue thereof and/or the protein encoded by the target sequence on the polynucleotide of the present invention. For example, the disease to be treated or prevented by the present method includes a central nervous system disease, a cognitive disorder, or a KCNH3-related disease, and is preferably ADHD, Parkinson's disease, Alzheimer's disease, or schizophrenia. Here, “KCNH3-related disease” refers to diseases caused by increased or decreased expression of KCNH3 (BEC1) or changes in its function. KCNH3 is a member of the K⁺ channel superfamily with a preferential forebrain distribution and a restricted expression distribution in the brain. Its expression is prominent in the hippocampus and cerebral cortex. In addition, hippocampus and cerebral cortex have been strongly suggested to be associated with memory and learning. Therefore, “KCNH3-related disease” includes diseases associated with cognitive decline, including decline in memory and learning.

In the method for treating or preventing a disease of the present invention, the polynucleotide of the present invention or a vector comprising the polynucleotide of the present invention are introduced into a subject, for example, into a cell of specific tissues or organs of the subject by using a known method in the art, for example, by using a known viral vector system or a nonviral vector system. The known introduction methods of polynucleotides or vectors can be used for the present method. The cell specificity may be controlled, for example, by promoter or other elements within a vector.

Due to the properties of the polynucleotide of the present invention, the expression of the protein encoded by the target sequence on the polynucleotide of the present invention can be regulated by the administration of ASP7967 or an analogue thereof into the subject. Here, examples of the protein encoded by the target sequence are mentioned above in the section of the polynucleotide of the present invention.

The delivery of the polynucleotide or vector comprising the target sequence and the delivery of the ligand, i.e., ASP7967 or an analogue thereof, generally are separated in time. The delivery of the ligand will control when the target gene is expressed, as well as the level of protein expression. The ligand may be delivered by a number of routes including, but not limited to, oral, intramuscular (IM), intravenous (IV), intraocular, or topically.

The timing of delivery of the ligand will depend on the requirement for activation or inactivation of the protein encoded by the target sequence. For example, if the protein encoded by the target sequence is required constantly for a therapy, the ligand may be delivered daily, or multiple times a day, to ensure continual activation or inactivation of that protein. If the protein has a long acting effect, the ligand may be delivered less frequently.

<Method for Treating a Disease in a Subject Who have Received a Gene Therapy>

The above method for treating or preventing a disease may be applied to a subject who have received a gene therapy. Accordingly, the present invention is related to the method for treating a disease, which comprises (2′) administering ASP7967 or an analogue thereof to a subject who have received a gene therapy with a vector comprising the polynucleotide of the present invention. Since the present method is applied to a subject who have received a gene therapy with a vector comprising the polynucleotide of the present invention, the step of (1) introducing the polynucleotide of the present invention or a vector comprising the polynucleotide of the present invention into a subject is optional.

In the present method, the subject have received a gene therapy with a vector comprising the polynucleotide of the present invention, e.g., using known gene therapy techniques. Here, “gene therapy” refers generally to the transfer of heterologous nucleic acids to a subject with a disorder or conditions for which such therapy is sought, for example, to a cell of specific tissues or organs of the subject. In the present invention, the vector comprising the polynucleotide of the present invention is introduced into the cells in a manner such that the polypeptide of the present invention is constantly or transiently expressed. Here, examples of the protein encoded by the target sequence in the polynucleotide of the present invention are mentioned above in the section of the polynucleotide of the present invention.

### EXAMPLES

### <Materials and Methods>

### 1. Aptamer Selection (SELEX)

### 1.1. Ligand-Coupled Sepharose Matrix

### 1.1.1. General

All chemicals and solvents (including dry DMF and dry dioxane) were purchased from commercial sources and were used without further purification. The intermediate compound (2) shown in Scheme 1 was obtained from BioFine. Thin-layer chromatography (TLC) was performed on silica gel plates pre-coated with fluorescence indicator and visualized by UV light (254 nm). Silica gel (45-75 μm) was used for column chromatography. ¹H NMR spectra were recorded on 400 MHz Bruker or 600 MHz JEOL NMR instrument. The chemical shifts (8) in parts per million were referenced to the residual proton signal (2.50 ppm) and carbon signal (39.5 ppm) of DMSO-d₆ for ¹H NMR and ¹³C NMR spectra respectively. Multiplicities of ¹H NMR spin couplings are reported as s (singlet), br s (broad singlet), d (doublet). Values for apparent coupling constants (J) are reported in Hz. High-resolution mass spectrometry (HRMS) data was obtained using the positive ion electrospray ionization (ESI) mode.

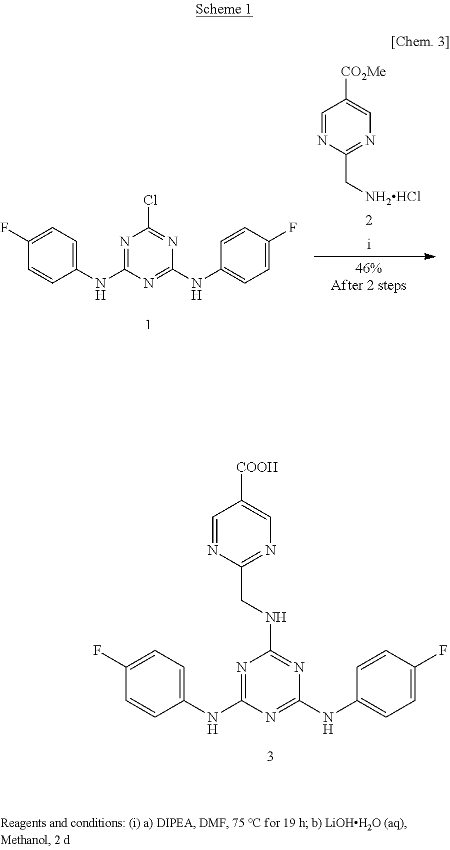

### 1.1.2. Synthesis of Compound 3

To a stirring solution of compound 1¹ (80 mg, 0.24 mmol) and compound 2 (61 mg, 0.30 mmol) in dry DMF (0.8 ml), DIPEA (209 μl, 1.2 mmol) was added under N₂ atmosphere. The resulting reaction mixture was heated to 75° C. for 19 h. Then, the reaction mixture was cooled to room temperature and subjected to column chromatography purification (35-80% ethyl acetate in hexane) to isolate the newly formed product (78 mg, 70%). The resulting compound (50 mg, 0.107 mmol) was dissolved in methanol (3 ml) to which a solution of LiOH·H₂O (55 mg, 1.31 mmol in 0.6 ml water) was added and stirred for 2 days. The volatiles were removed and water (2 ml) was added to the resulting compound. The pH was adjusted to ~7-8 with 2 N HCl, and the formed precipitate was filtered and the residue was washed with water followed by acetone and diethyl ether, which furnished the desired compound 3 (32 mg, 66%).

¹H NMR (400 MHz, DMSO-d₆): ¹H NMR (400 MHz, DMSO-d₆) δ 9.17 (s, 2H), 9.11 (br s, 2H), 7.79-7.55 (m, 5H), 6.99-7.12 (m, 4H), 4.76 (d, J=5.2 Hz, 2H); ¹³C NMR (150 MHz, DMSO-d₆): δ 170.7, 165.8, 165.0, 163.9, 158.0, 157.3 (J=238.5 Hz), 136.4, 121.5, 114.8 (J=21.0 Hz), 114.5, 46.3; ¹⁹F NMR (376 MHz, DMSO-d₆) δ −121.7; HRMS (m/z): [M+H]⁺ calcd for C₂₁H₁₇F₂NgO₂⁺451.1437, found 451.1435.

### 1.1.3. Immobilization of Compound 3 to Sepharose Beads

EAH Sepharose 4B (Cytiva) containing free amino groups was coupled with compound 3 using PyBOP (benzotriazole-1-yl-oxy-tris-pyrrolidino-phosphonium hexafluorophosphate). Sepharose 4B (6 ml) was transferred to a 10 ml column (PD-10 Cytiva) and the ethanol solution was drained off. The matrix was washed twice with DMF (6 ml each). Subsequently, compound 3 (6.2 mg, 13.76 μmol in 500 μl dry DMF), PyBOP (9.36 mg, 17.98 μmol in 500 μl DMF), and DIPEA (5 μl, 28.7 μmol) were added. The column containing the reaction mixture was placed in a shaker for 3 h at 25° C. Then, the unreacted compounds were drained off from the matrix. The matrix was washed with DMF (2×1 ml) and acetonitrile (2×6 ml). The unreacted free amino groups of the matrix were blocked by acetyl groups by treating it with Cap A solution (THF:Lutidine:Acetic anhydride [8:1:1]; 6 ml) for 30 min at 25° C. Then, the solution was drained off from the matrix, which was washed with acetonitrile (3×6 ml). Finally, the matrix was stored in 50% methanol in water (6 ml) at 4° C. Similarly, acetylated matrix was prepared by directly treating Sepharose 4B (6 ml) with Cap A solution for negative selection.

### 1.2. Oligonucleotides, Molecular Biology, and Buffer Reagents

OneTaq 2× Master Mix with Standard Buffer (NEB) and Q5 High-Fidelity 2× Master Mix (NEB) were used for PCR in SELEX rounds and NGS sequencing library preparation, respectively. Reverse transcription reactions were performed using Superscript III Reverse Transcriptase (Thermo Scientific) with Rev-primer (Table 1). HiScribe T7 Quick High Yield RNA Synthesis Kit (NEB) was used for in vitro transcription of the initial RNA pool (700 μl) as well as for the subsequent SELEX rounds (20 μl scale).

TABLE 1
List of oligonucleotides
used for SELEX experiments

| Code | Sequence (5′-3′) | Length |
|---|---|---|
| N40-T7Fwdª | TTCAGGTAATACGACTCACTA | 44 nt |
| | TAGGGAAGAGAAGGACATATG | |
| | AT (SEQ ID NO: 28) | |
| N40-Rev-Lib^{b} | TCAAGTGGTCATGTACTAGTC | 86 nt |
| | AANNNNNNNNNNNNNNNNNNN | |
| | NNNNNNNNNNNNNNNNNNNNN | |
| | ATCATATGTCCTTCTCTTCCC | |
| | TA (SEQ ID NO: 29) | |
| Rev-Primer^{c} | TCAAGTGGTCATGTACTAGTC | 23 nt |
| | AA (SEQ ID NO: 30) | |

^{a,c}Used for PCR during SELEX rounds.
^{b}N indicates mixture of A, T, G and C (25% each).
^{c}Used for reverse transcription.

SELEX buffer composed of and 10 mM HEPES-KOH (pH 7.4), 140 mM KCl, 10 mM NaCl, 1 mM MgCl₂, 5% (v/v) DMSO, and 0.01% (v/v) Tween 20 was used for refolding, washing, and elution. Only in the case of elution, the buffer was supplemented with 1 mM ASP7967.

### 1.3. Aptamer Selection (SELEX)

Overlap extension of N₄₀-T7Fwd (1 nmol) and N₄₀-Rev-Lib (1 nmol; ~6×10¹⁵ unique sequences) (Table 1) was performed in 1.0 ml volume that consists of 1× standard (Mg-free) reaction buffer, 2 mM MgCl₂, 0.2 mM dNTPs, and Taq DNA polymerase (25 U, NEB) for 2.5 min at 94° C., 30 s at 49° C., and 5.5 min at 68° C. Subsequently, the dsDNA was recovered by ethanol precipitation. The dsDNA containing a T7 promoter (38 μg, ~0.57 nmol) was in vitro transcribed in 700 μl volume for 6 h at 37° C. using HiScribe T7 Quick High Yield RNA Synthesis Kit (NEB). The solution was treated with 80 μl of 10× DNase I buffer and 20 μl of DNase I (40 U, NEB) for 45 min at 37° C. The RNA pool was recovered by phenolchloroform extraction followed by ethanol precipitation to be used as the initial RNA pool for SELEX.

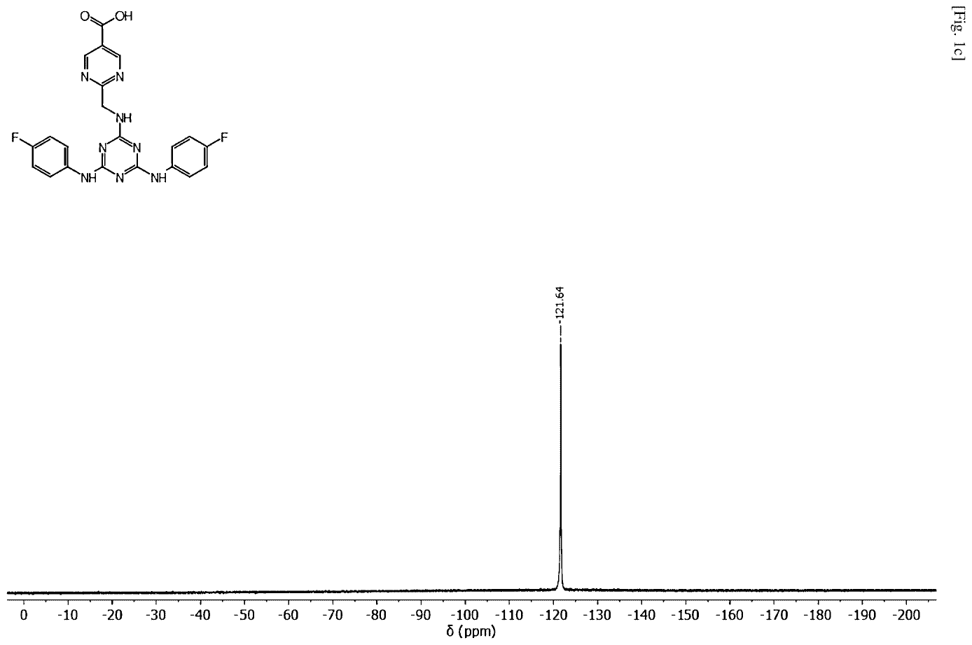

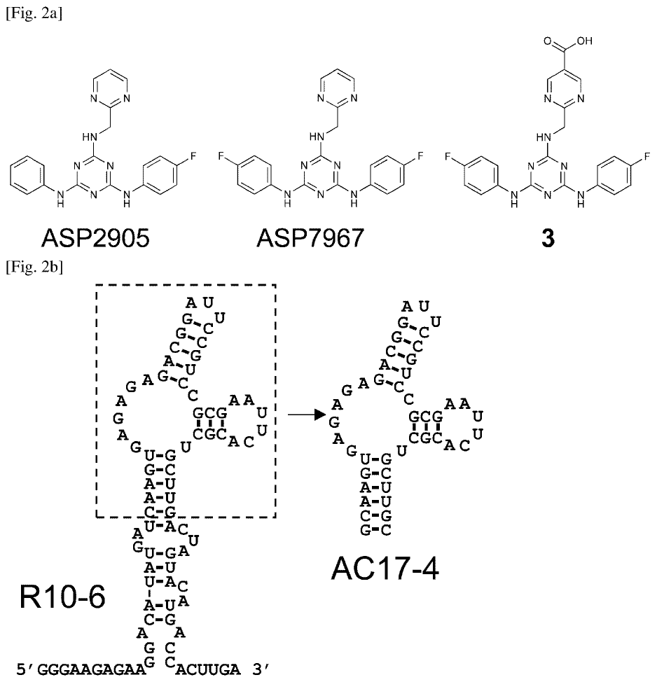

The initial RNA pool (115.8 μg, ~4.2 nmol) was folded in 1.2 ml SELEX buffer (10 mM HEPES-KOH, pH 7.4, 140 mM KCl, 10 mM NaCl, 1 mM MgCl₂, 5% (v/v) DMSO, 0.01% (v/v) Tween 20) by incubating at 80° C. for 3 min followed by cooling on ice. The annealed RNA library pool was incubated with 200 μl of the ligand-coupled Sepharose matrix in a column (PD-10, Cytiva) for 45 min at 25° C. while shaking (Table 2). Subsequently, the unbound RNAs were removed from the column by gravity. The matrix was washed with SELEX buffer twice (1+0.4 ml), and the bound RNA sequences were recovered by adding 0.2 ml SELEX buffer supplemented with ASP7967 (1 mM) after shaking for 30 min at 25° C. The elution was repeated one more time. The eluted RNAs were ethanol precipitated using Quick-Precip Plus Solution (EdgeBio). The progress of SELEX experiments was monitored by measuring absorbance (260 nm) of the recovered RNAs (Table 3). The RNA pool then was then reverse transcribed with Rev-Primer (Table 1) using SuperScript III Reverse Tran-scriptase (Thermo Fisher Scientific) and amplified by PCR with N40-T7Fwd and Rev-Primer (Table 1) using OneTaq 2× Master Mix with Standard Buffer (NEB). The PCR product was used as a template for in vitro transcription, treated with DNase I, and ethanol precipitated to produce the RNA pool for the next round of SELEX. In the subsequent rounds of SELEX, various parameters such as the amount of input RNA pool, temperature, volume of washing buffer, elution time, etc. were altered to adjust the stringency of selection (Table 2). From the 4th round, negative selection was performed by incubating the input RNA pool with acetyl-capped matrix (150-1000 μl) prior to incubation with the ligand-immobilized matrix to select against sequences that have affinity to the gel matrix (Table 2). The RNA pools generated after the rounds 7, 8, 9 and 10 were used to prepare libraries for deep sequencing analysis. The RNAs from each round were reverse transcribed (with a unique barcode sequence to identify the round) and PCR (with adapter sequences) amplified individually. The resulting sequencing libraries were pooled together and sequenced using MiSeq Reagent Kit v3 (Illumina). Eleven sequences were chosen for ITC measurement (data not shown) based on the enrichment efficiency in the last three rounds of SELEX. R10-6 (5′-GGGAAGAGAAGGACAUAUGAUCAAGUGAGAGAGACGGAUUCCGUCC GCGAAUUCACGCUGCUUGACUAGUACAUGACCACUUGA-3′ (SEQ ID NO: 31)) (FIG. 2b) was selected for further analysis due to its strong affinity observed by ITC (data not shown).

TABLE 2
Experimental conditions used in SELEX

| | Amount of | Volume of acetyl-capped | Volume of ligand- | | | Volume of ligand (1 |
|---|---|---|---|---|---|---|
| | input RNA | beads and incubation time | Immobilized beads and | Temper- | Volume of wash buffer | mM) elution solution |
| Round | pool in 1.2 ml | for negative selection | incubation time (min) | ature | and incubation time | and incubation time |
| 1 | 4.20 nmol | No | 200 μl | 25 °C | 1.4 ml (1 + 0.4 ml) | 0.4 ml (0.2 ml × 2) |
| | | | (45 min) | | 10 min each | 30 min each |
| 2 | 2.89 nmol | No | 200 μl | 25 °C | 1.6 ml (1 + 0.6 ml) | 0.4 ml (0.2 ml × 2) |
| | | | (30 min) | | 10 min each | 30 min each |
| 3 | 2.75 nmol | No | 200 μl | 25 °C | 1.8 ml (1.2 + 0.6 ml) | 0.4 ml (0.2 ml × 2) |
| | | | (30 min) | | 10 min each | 30 min each |
| 4 | 2.51 nmol | 150 μl | 150 μl | 25 °C | 2.2 ml (1.2 + 1.0 ml) | 0.4 ml (0.2 ml × 2) |
| | | (30 min) | (15 min) | | 10 min each | 15 min each |
| 5 | 2.22 nmol | 200 μl | 150 μl | 25 °C | 2.6 ml (1.2 + 1.4 ml) | 0.4 ml (0.2 ml × 2) |
| | | (30 min) | (15 min) | | 10 min each | 15 min each |
| 6 | 1.75 nmol | 200 μl | 150 μl | 25 °C | 3 ml (1.2 + 1.2 + 0.6 ml) | 0.4 ml (0.2 ml × 2) |
| | | (30 min) | (10 min) | | 10 min each | 10 min each |
| 7 | 1.29 nmol | 200 μl | 125 μl | 25 °C | 3.4 ml (1.2 + 1.2 + 1 ml) | 0.4 ml (0.2 ml × 2) |
| | | (30 min) | (10 min) | | 20 min each | 10 min each |
| 8 | 1.12 nmol | 250 μl | 125 μl | 37 °C | 3.6 ml (1.2 ml × 3) | 0.4 ml (0.2 ml × 2) |
| | | (30 min) | (10 min) | | 20 min each | 10 min each |
| 9 | 0.61 nmol | 600 μl | 125 μl | 37 °C | 4.8 ml (1.2 ml × 4) | 0.4 ml (0.2 ml × 2) |
| | | (30 min) | (10 min) | | 20 min each | 10 min each |
| 10 | 0.27 nmol | 1000 μl | 60 μl | 37 °C | 7.2 ml (1.2 ml × 6) | 0.4 ml (0.2 ml × 2) |
| | | (60 min) | (7 min) | | 25 min each | 7 min each |

TABLE 3
Amount of RNA pool eluted after each SELEX round

| Round | Percentage of input RNA eluted |
|---|---|
| 1 | 0.53 |
| 2 | 0.7 |
| 3 | 1.02 |
| 4 | 0.88 |
| 5 | 0.67 |
| 6 | 1.2 |
| 7 | 2.53 |
| 8 | 2.71 |
| 9 | 10.4 |
| 10 | 4.1 |

### 2. Affinity Measurement by SPR

Affinity measurements of the aptamers with the ligands by SPR were performed as described by Chang et al.⁵³ with some modifications. Experiments were performed on Biacore T200 (Cytiva) at 25° C. For immobilization of the capture DNA (5′-/5AmMC6/TTTTTTTTTTTTTTTTTTTTTTTT-3′ (SEQ ID NO: 32),/5AmMC6/: 5′ amino modifier C6, IDT), 1×HBS-N (10 mM HEPES, pH 7.4, 150 mM NaCl) was used as the running buffer. CM5 chip surface was washed by two injections of NaOH/NaCl solution (25 mM NaOH, 1 M NaCl) at 20 μl min⁻¹ for 30 s. Next, a solution containing 200 mM 1-ethyl-3-(3-dimethylaminopropyl) carbodiimide hydrochloride (EDC) and 50 mM N-hydroxysuccinimide (NHS) was injected into all flow cells at 10 μl min⁻¹ for 7 min to activate the surface carboxylic acid groups. Then, the capture DNA (20 μM) in 10 mM HEPES-KOH buffer (pH 7.5) supplemented with 0.6 mM cetyltrimethylammonium bromide was injected at 5 μl min⁻¹ for 10 min. The unreacted activated carboxylic acid groups were quenched by an injection of 1 M ethanolamine-HCl (pH 8.5) for 7 min at 10 μl min⁻¹. To remove the noncovalently adsorbed DNA on the chip surface, NaOH/NaCl solution was injected two times at 20 μl min⁻¹ for 30 s. The immobilized DNA levels were 2846±284 RU.

The dsDNA templates for in vitro transcription of the aptamers were prepared by primer extension of two oligo DNAs using Q5 High-Fidelity DNA Polymerase (NEB). AC17-4 RNA aptamer and its mutants containing 3′ poly(A) tail (Table 4) were synthesized by in vitro transcription using ScriptMAX Thermo T7 Transcription Kit (TOYOBO) according to the manufacturer's instructions. The reaction products were treated with 2 U TURBO DNase (Thermo Fisher Scientific) for 30 min at 37° C. and purified with RNA Clean & Concentrator-25 Kit (Zymo Research). RNA concentrations were determined by absorbance at 260 nm according to OligoCalc⁵⁴. RNA solutions (~3.6 μM) were prepared in water and denatured by heating at 80° C. for 3 min. After cooling at room temperature for few minutes, the RNA solutions were diluted with an equal volume of high salt buffer (10 mM Tris-HCl, pH 7.5, 1 M NaCl, 1 mM EDTA). Stock solutions of ASP2905 and ASP7967 in DMSO (20 mM, fumaric acid adduct, Astellas Pharma, Inc.) were prepared based on the weight. Various concentrations of the ligand solutions were prepared in SPR running buffer (10 mM HEPES, pH 7.4, 150 mM NaCl, 0.05% (v/v) surfactant P-20, 1 mM MgCl₂, 2% (v/v) DMSO). An RNA solution was injected at 10 μl min⁻¹ for 30 s followed by an injection of the SPR running buffer for 20 s. An aptamer ligand was injected at 30 μl min⁻¹ for 120 s to monitor association, and then the dissociation kinetics was monitored for 180 s in the SPR running buffer. The sensor surface was regenerated with a 10 μl injection of 25 mM NaOH at 30 μl min⁻¹ followed by the SPR running buffer for 30 s. The raw data were analysed by Biacore T200 Evaluation Software 1.0 using 1:1 Langmuir interaction model. Background signal from the reference flow cell was subtracted from that of the sample flow cell, and no-ligand sample (SPR running buffer only) was injected in each experiment (double referencing). Dissociation constant (K_{D}) was determined from the ratio of the association and the dissociation rate constants (K_{D}=k_{off}/kₒₙ) or by equilibrium analysis. The figures were generated using GraphPad Prism 9. The measurements were repeated at least twice to ensure reproducibility.

TABLE 4
Sequences of AC17-4 aptamer
and its mutants analyzed by SPR

| Aptamer | Sequence (5′-3′) |
|---|---|
| AC17-4 | GCAAGUGAGAGAGACGGAUUCCGUCCGCGAAU |
| (WT) | UCACGCUGCUUGCAAAAAAAAAAAAAAAAAAA |
| | AAAAA (SEQ ID NO: 33) |
| M1 | GCAAGUCAGAGAGACGGAUUCCGUCCGCGAAU |
| | UCACGCUGCUUGCAAAAAAAAAAAAAAAAAAA |
| | AAAAA (SEQ ID NO: 34) |
| M2 | GCAAGUGUGAGAGACGGAUUCCGUCCGCGAAU |
| | UCACGCUGCUUGCAAAAAAAAAAAAAAAAAAA |
| | AAAAA (SEQ ID NO: 35) |
| M3 | GCAAGUGACAGAGACGGAUUCCGUCCGCGAAU |
| | UCACGCUGCUUGCAAAAAAAAAAAAAAAAAAA |
| | AAAAA (SEQ ID NO: 36) |
| M4 | GCAAGUGAGUGAGACGGAUUCCGUCCGCGAAU |
| | UCACGCUGCUUGCAAAAAAAAAAAAAAAAAAA |
| | AAAAA (SEQ ID NO: 37) |
| M5 | GCAAGUGAGACAGACGGAUUCCGUCCGCGAAU |
| | UCACGCUGCUUGCAAAAAAAAAAAAAAAAAAA |
| | AAAAA (SEQ ID NO: 38) |
| M6 | GCAAGUGAGAGUGACGGAUUCCGUCCGCGAAU |
| | UCACGCUGCUUGCAAAAAAAAAAAAAAAAAAA |
| | AAAAA (SEQ ID NO: 39) |
| M7 | GCAAGUGAGAGAGACGGAUUCCGUCAGCGAAU |
| | UCACGCUGCUUGCAAAAAAAAAAAAAAAAAAA |
| | AAAAA (SEQ ID NO: 40) |
| M8 | GCAAGUGAGAGAGACGGAUUCCGUCCGCGAAU |
| | UCACGCGGCUUGCAAAAAAAAAAAAAAAAAAA |
| | AAAAA (SEQ ID NO: 41) |
| M9 | GCAAGGGAGAGAGACGGAUUCCGUCCGCGAAU |
| | UCACGCUUCUUGCAAAAAAAAAAAAAAAAAAA |
| | AAAAA (SEQ ID NO: 42) |
| M10 | GCAAGUGAGAGACACGGAUUCCGUGCGCGAAU |
| | UCACGCUGCUUGCAAAAAAAAAAAAAAAAAAA |
| | AAAAA (SEQ ID NO: 43) |
| M11 | GCAAGUGAGAGAGACGGAUUCCGUCCCCGAAU |
| | UCACGGUGCUUGCAAAAAAAAAAAAAAAAAAA |
| | AAAAA (SEQ ID NO: 44) |
| M12 | GCAAGUGAGAGAGACGGUUCGCCGUCCGCGAA |
| | UUCACGCUGCUUGCAAAAAAAAAAAAAAAAAA |
| | AAAAAA (SEQ ID NO: 45) |
| M13 | GCAAGUGAGAGAGACGGAUUCCGUCCGCGUUU |
| | CGACGCUGCUUGCAAAAAAAAAAAAAAAAAAA |
| | AAAAA (SEQ ID NO: 46) |
| M14 | GCAAGUGAGAGAGACGGAUUCCGUCCGCGAAU |
| | UCACGCCGCUUGCAAAAAAAAAAAAAAAAAAA |
| | AAAAA (SEQ ID NO: 47) |
| M15 | GCAAGUGAGAGAGACGGAUUCCGUCCGCGAAU |
| | UCUCGCUGCUUGCAAAAAAAAAAAAAAAAAAA |
| | AAAAA (SEQ ID NO: 48) |
| M16 | GCAAGUGAGAGAGACGGAUUCCGUCCGCGUAU |
| | UCACGCUGCUUGCAAAAAAAAAAAAAAAAAAA |
| | AAAAA (SEQ ID NO: 49) |

Mutated sequence is underlined.

### 3. Affinity Measurement by ITC

ITC experiments were carried out as described previously²⁶ with some modifications. Template DNA for in vitro transcription was prepared by primer extension using Q5 High-Fidelity DNA Polymerase. AC17-4 RNA aptamer (5′-GCAAGUGAGAGAGACGGAUUCCGUCCGCGAAUUCACGCUGCUUGC-3′ (SEQ ID NO: 50)) was prepared using HiScribe T7 High Yield RNA Synthesis Kit (NEB) according to the manufacturer's instructions. The transcription product (100 μl) was treated with 2 U TURBO DNase at 37° C. for 30 min. The RNA was precipitated with ammonium acetate and ethanol and dissolved in water. The solution was further extracted with phenol-chloroform and ethanol precipitated. The RNA was purified by denaturing polyacrylamide gel electrophoresis (PAGE) and the main band was extracted from the gel with TE buffer (10 mM Tris-HCl, pH 7.0, 0.1 mM EDTA). The purified RNA was concentrated and the buffer was exchanged to nuclease-free water using an ultrafiltration device (Amicon Ultra 0.5 ml, 3 kDa, Merck-Millipore). RNA concentration was determined by absorbance at 260 nm according to OligoCalc. RNA was mixed with 15 μl of DMSO and diluted to 270 μl with nuclease-free water, denatured at 80° C. for 3 min, and incubated at room temperature for 5 min. After addition of 30 μl of 10×ITC buffer (0.2 M HEPES-KOH, pH 7.5, 1.4 M KCl, 0.1 M NaCl, 10 mM MgCl₂), the 7.5 μM RNA solution (300 μl) was incubated at room temperature for 30 min before measurement. ASP2905 solution (75 μM) was prepared in 5% (v/v) DMSO-1×ITC buffer just before the measurement.

Titration was performed at 37° C. using MicroCal PEAQ-ITC (Malvern). Injection parameters were as follows: initial 300 s delay, single 0.4 μl injection, and 24 serial injections of 1.5 μl at intervals of 120 s. Stirring speed and reference power were set to 750 rpm and 5 μcal s⁻¹, respectively. Raw data were analysed by MicroCal PEAQ-ITC analysis software ver. 1.0.0.1259 using one-site binding model. Measurements from the control titrations (ASP2905 into buffer, buffer into AC17-4 RNA aptamer, buffer into buffer) were subtracted from the sample measurements. The measurements were repeated twice to ensure reproducibility.

### 4. Riboswitch Plasmid Design and Construction

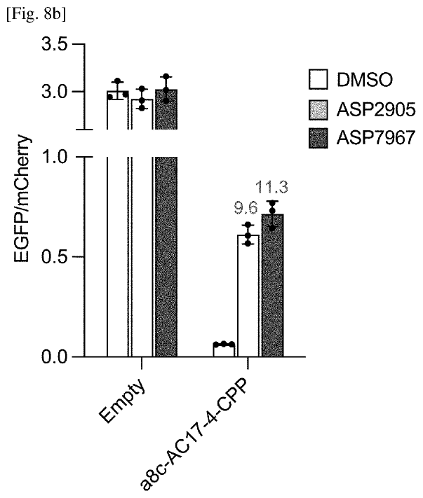

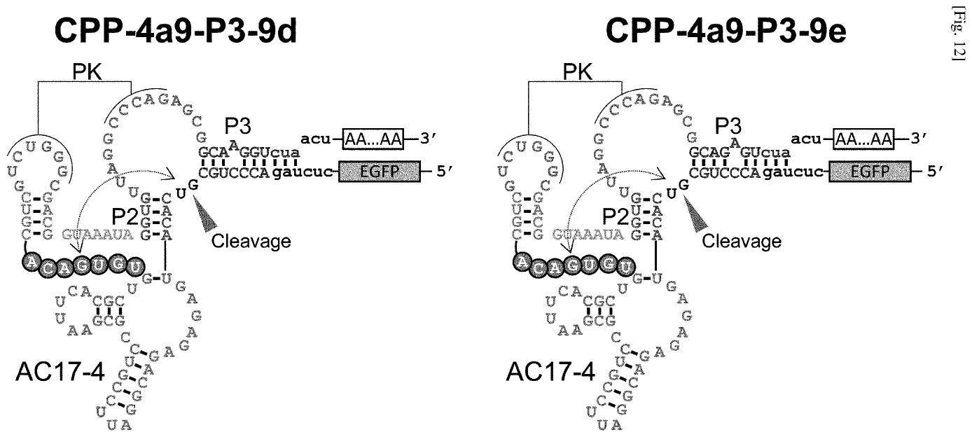

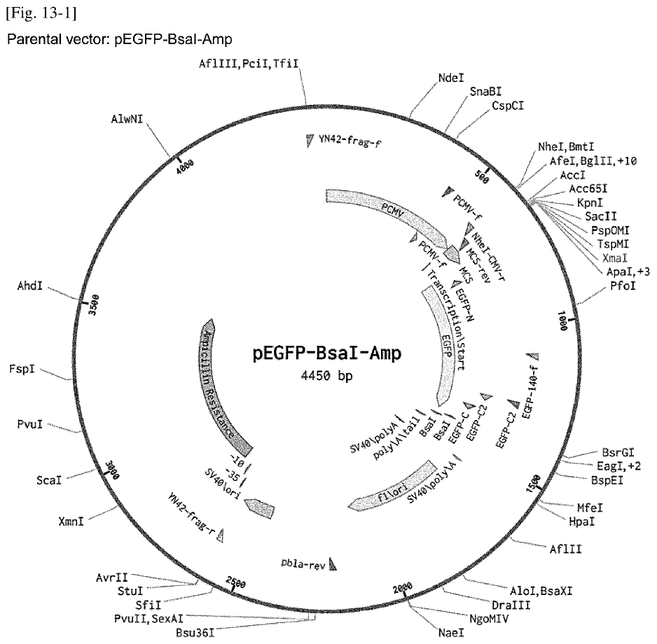

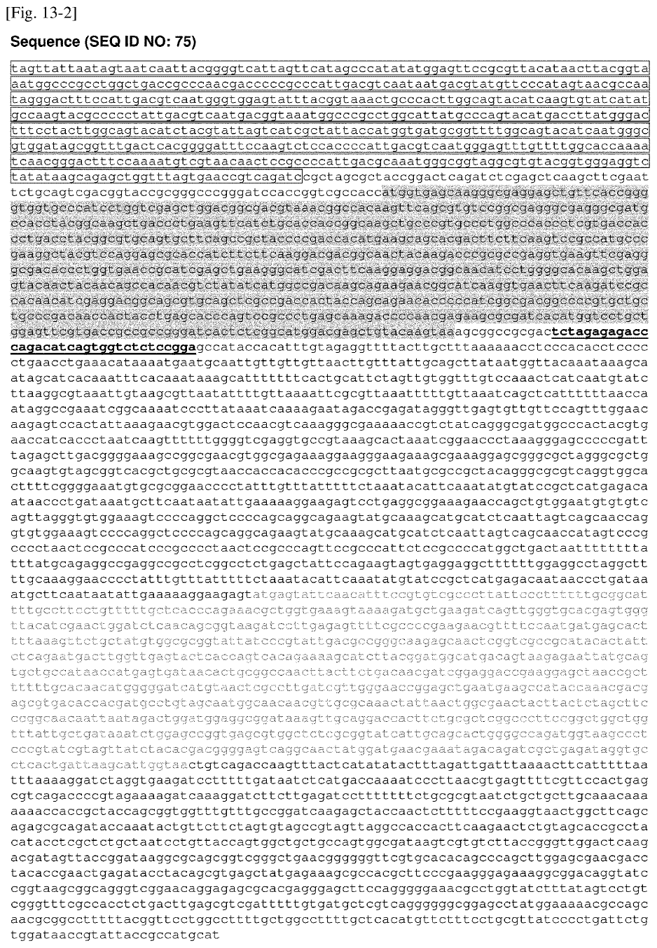

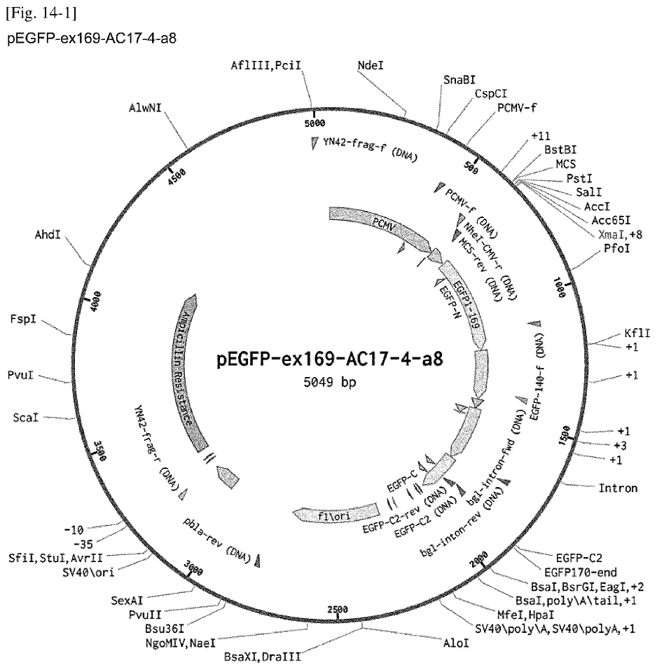

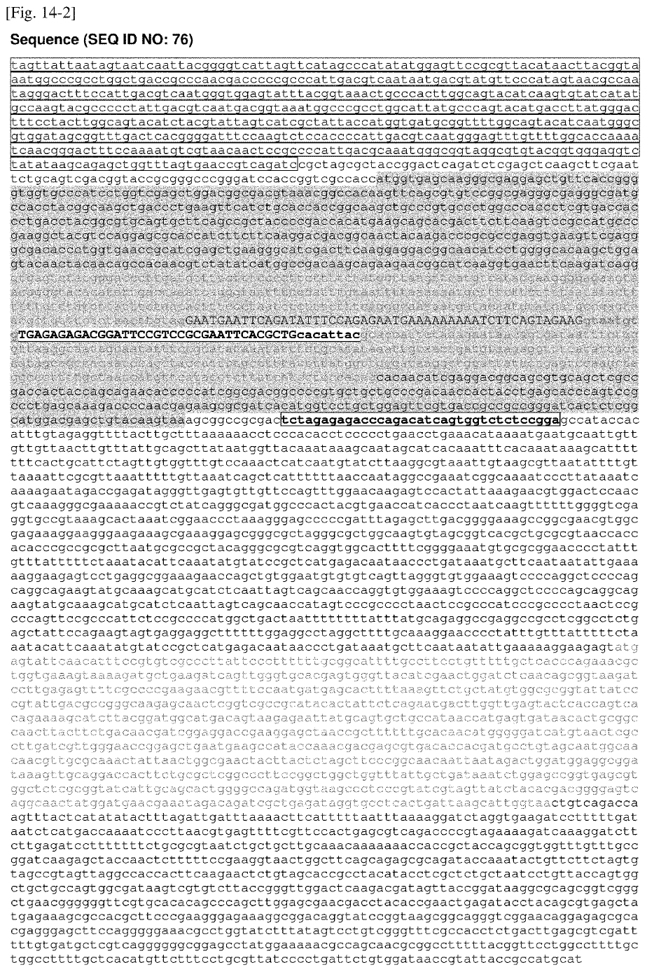

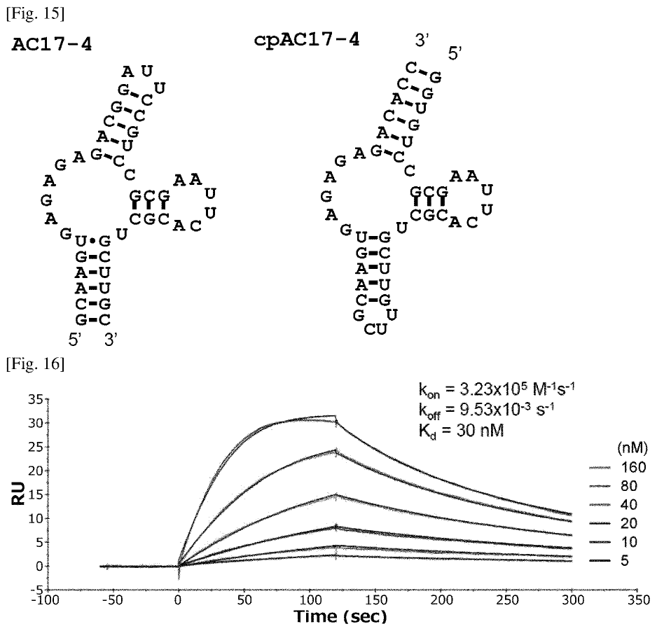

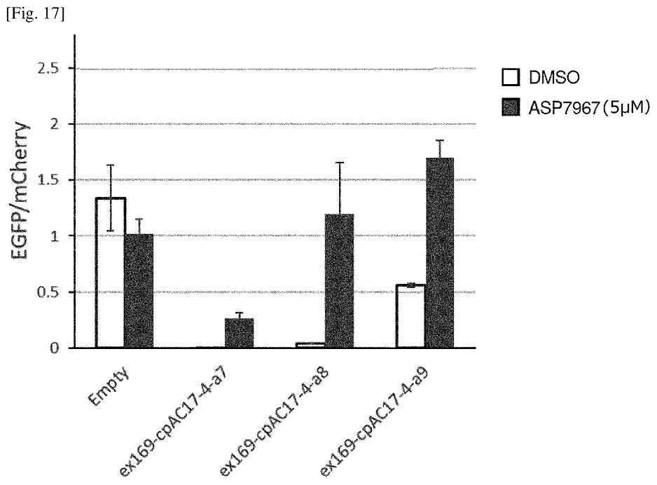

Aptazyme sequences shown in FIG. 6b, FIG. 8a, and FIG. 12 were cloned into the 3′ UTR of the EGFP mRNA encoded in pEGFP-BsaI-Amp (FIG. 13). The sequences are presented in Table 5. An exon-skipping riboswitch cassette was inserted between the 169th and 170th codons in the EGFP coding sequence in pEGFP-BsaI-Amp. The full plasmid sequence for ex169-AC17-4-a8 (pEGFP-ex-169-AC17-4-a8) is provided in FIG. 14. Other variant sequences are listed in Table 6. pEGFP-BsaI-Amp was used as an “empty vector” control in the transfection experiments.

TABLE 5
Table 5. Riboswitch sequences (aptazyme)

| Riboswitch | Sequence (5′-3′)¹ |
|---|---|
| a6-AC17-4-CPP | 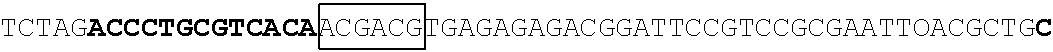 |
| | GTCGTCTGGGCGACGGTAAATAGGTGTTAGGCCCAGAGCGGCAGGGTACAACTCCGGA |
| | (SEQ ID NO: 51) |
| a7-AC17-4-CPP | 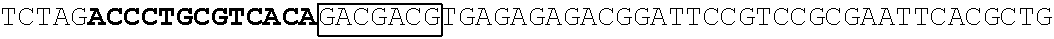 |
| | CGTCGTCTGGGCGACGGTAAATAGGTGTTAGGCCCAGAGCGGCAGGGTACAACTCCGGA |
| | (SEQ ID NO: 52) |
| a8-AC17-4-CPP | 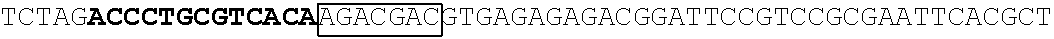 |
| | 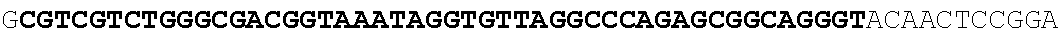 |
| | (SEQ ID NO: 53) |
| a9-AC17-4-CPP | 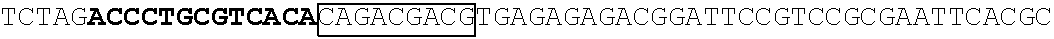 |
| | 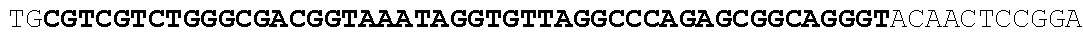 |
| | (SEQ ID NO: 54) |
| a10-AC17-4-CPP | 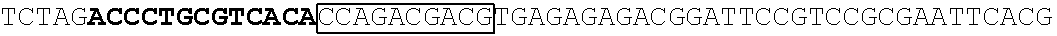 |
| | 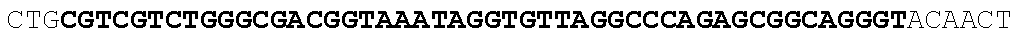 |
| | (SEQ ID NO: 55) |
| a11-AC17-4-CPP | 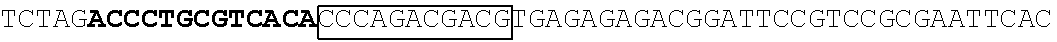 |
| | 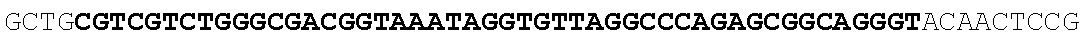 |
| | GA (SEQ ID NO: 56) |
| a8c-AC17-4- | 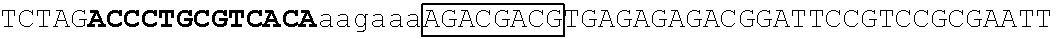 |
| CPP² | 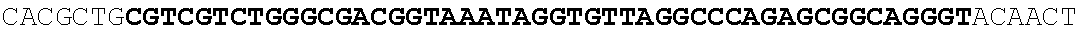 |
| | CCGGA (SEQ ID NO: 57) |
| CPP-AC17-4-a9- | 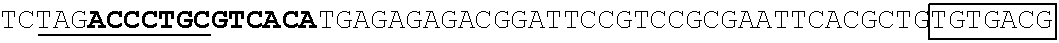 |
| P3-9d³ | 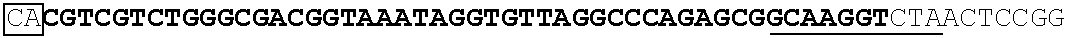 |
| | A (SEQ ID NO: 58) |
| CPP-AC17-4-a9- | 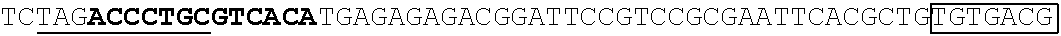 |
| P3-9e³ | 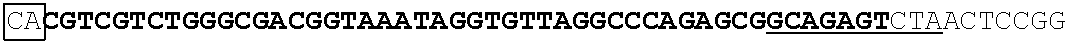 |
| | A (SEQ ID NO: 59) |

¹Sequences are shown in DNA. CPP ribozyme sequence is shown in bold, anti-Rz is shown with box, and AC17-4 aptamer is shown with shading.
²The 6-nt spacer upstream of the anti-Rz sequence in a8c-AC17-4-CPP is depicted in lowercase letters.
³Extended P3 stem sequences are underlined. These constructs contain an anti-Rz sequence downstream of the aptamer that interfere with P2 and P3 stems.

TABLE 6
Table 6. Riboswitch sequences (exon-skipping)

| Riboswitch | Sequence (5′-3′)¹ |
|---|---|
| ex169-AC17-4-a7 | 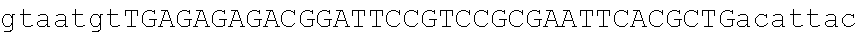 |
| | (SEQ ID NO: 60) |
| ex169-AC17-4-a8 | 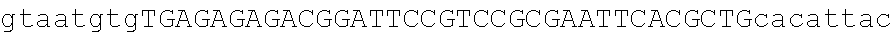 |
| | (SEQ ID NO: 61) |
| ex169-AC17-4-a9 | 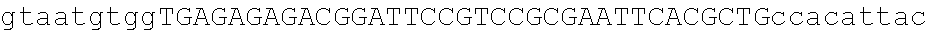 |
| | (SEQ ID NO: 62) |
| ex169-AC17-4- | 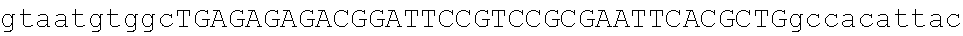 |
| a10 | (SEQ ID NO: 63) |
| ex169-AC17-4- | 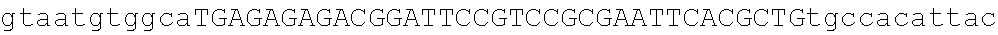 |
| a11 | (SEQ ID NO: 64) |
| ex169-AC17-4- | 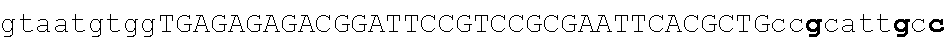 |
| a9+g2g7 | (SEQ ID NO: 65) |

¹Sequences are shown in DNA. AC17-4 aptamer is shown with shading.

### 5. Riboswitch Assay in HEK293 Cells

HEK293 cells were cultured in Dulbecco's modified Eagle's medium (DMEM) supplemented with 10% heat-inactivated FBS (Gibco) containing 2 mM L-glutamine and 100 units ml⁻¹ of penicillin-streptomycin (DMEM-FBS). Cells were kept in a 37° C. incubator with 5% CO₂ and passaged regularly upon reaching 90% confluency. Approximately 20 h prior to transfection, the cells were trypsinized, diluted to ~2.7×10⁵ cells ml⁻¹ and 100 μl per well were seeded onto a 96-well plate. Cells in each well were cotransfected with 100 ng of the EGFP-aptazyme plasmid and 20 ng of pCMV-mCherry²⁷ (transfection control) using 0.3 μl of TransIT-293 Transfection Reagent (Mirus) according to the manufacturer's instructions. Five hours after transfection, the medium in each well was replaced with fresh medium with (up to 5 μM) or without ASP2905 or ASP7967. The aptamer ligands were dissolved at 1000× concentration in DMSO. Forty-eight hours after transfection, the medium in each well was replaced with 100 μl of phosphate buffered saline (PBS), and fluorescence intensity was measured by Infinite M1000 PRO microplate reader (Tecan). Fluorescence intensity was measured at 484 nm excitation/510 nm emission/5 nm bandwidth for EGFP, and at 587 nm excitation/610 nm emission/10 nm bandwidth for mCherry. Background fluorescence measured using untransfected cells was subtracted from the EGFP and mCherry fluorescence values. Then, EGFP fluorescence was normalized by mCherry fluorescence to account for variations in transfection efficiency and cell counts. All reported values are averages of three replicate wells.

### 6. MTT Assay

MTT assay was performed using MTT Cell Count Kit (Nacalai Tesque). HEK293 cells were trypsinized, diluted to 2.4×10⁵ cells ml⁻¹ and 100 μl per well were seeded onto a 96-well plate. The cells were cultured for 24 h at 37° C. under 5% CO₂. The medium was replaced with fresh medium (DMEM-FBS with 0.1% (v/v) DMSO) with 0, 2, 5, or 10 μM of ASP2905 or ASP7967. The cells were cultured for additional 18 h at 37° C. under 5% CO₂. Subsequently, 10 μl of MTT solution was added to each well, and the cells were incubated for 3 h at 37° C. under 5% CO₂. Next, 100 μl of the Solubilization Solution was added to each well. To dissolve the precipitated formazan, the plate was incubated for 2 h at 37° C. Absorbance at 570 nm (reference wavelength: 700 nm) was measured by Infinite M1000 PRO microplate reader (Tecan). The reported values are averages of four replicate wells. The assay was repeated three times to ensure reproducibility.

7. In Vitro hEPO Assay

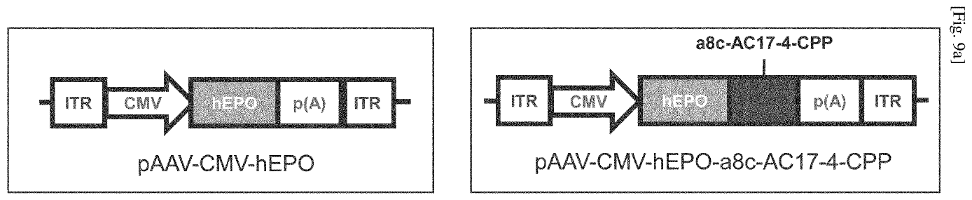

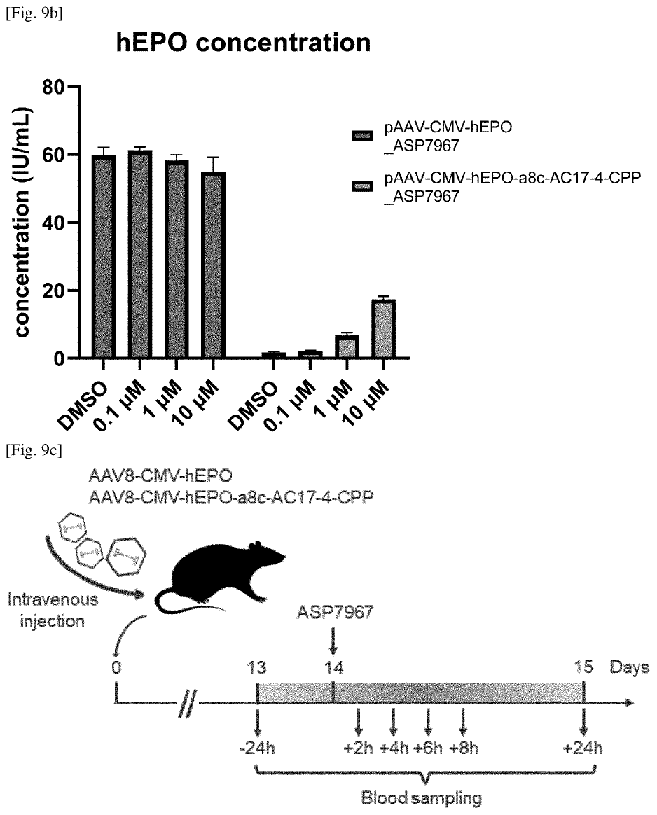

For the hEPO ELISA assay in FIG. 9b, HEK293 cells were trypsinized and diluted to 2.0×10⁵ cells ml⁻¹ and 100 μl per well were seeded onto a 96-well collagen plate. Cells in each well were transfected with 100 ng of the pAAV-CMV-hEPO-p3-d pAAV-CMV-hEPO-control, pAAV-MCS-based plasmids encoding human EPO with or without CPP-a8c-AC17-4 riboswitch using 0.3 μl of TransIT-293 Transfection Reagent according to the manufacturer's instructions. The medium in each well was replaced with fresh medium with (up to 10 μM) or without ASP7967 5 h after transfection. At 24 h after transfection, the medium in each well was collected and stored at −20° C. To measure hEPO concentrations, the medium was diluted 50-fold by PBS and further diluted 50-fold by the specimen diluent buffer included in the ELISA kit. The diluted samples were measured using Human Erythropoietin/EPO Quantikine ELISA Kit (R&D systems, Inc.) and Infinite M200 PRO microplate reader (TECAN).

8. In Vivo hEPO Assay

For AAV production, 293T cells seeded in cellstack5 (Corning) were cotransfected using PEI MAX (Polysciences Inc) with 227.9 μg of the plasmid encoding AAV8 replication and capsid proteins, 455.8 μg of the plasmid encoding helper proteins and 227.9 μg of the pAAV-CMV-hEPO-a8c or -control. At 6 days after transfection, cells and supernatants were harvested, then filtered and concentrated by KrosFlo Research Ili (Spectrum Labs, Inc) for AAV purification by affinity chromatography using AKTA avant 25 (GE healthcare). Then, the virus solutions were ultracentrifuged and dialyzed. Titers of the AAV solutions were determined by quantitative PCR using the AAVpro Titration Kit (for Real-Time PCR) Ver.2 (Takara).

For in vivo animal experiments, 7-week-old male BALB/c cAJc1 mice (CLEA Japan Inc.) were intravenously injected with 200 μl of PBS (as the control group) or purified AAV8 particles (3.0×10¹⁰ virus genome (vg) per mouse) carrying hEPO gene with or without the CPP-a8c-AC17-4 riboswitch. After 13 days from AAV injection, mice were subjected to 24 μl blood collection from tail vein and the blood samples were immediately diluted with 96 μl. At 24 h after the blood collection, the mice were orally administrated 100 mg kg⁻¹ of ASP7967 in 0.5% methyl cellulose (FUJIFILM Wako Pure Chemical Corp) or 0.5% methyl cellulose as the vehicle solution. At 2, 4, 6, 8 and 24 h after the ASP7967 administration, blood samples were collected as described above. The blood samples were incubated on ice, then centrifuged at 1,200×g for 15 min at 4° C. to collect serum samples. The serum samples were stored at −80° C. and used for measurement of serum hEPO concentration by ELISA.

All animal experimental procedures were approved by the Institutional Animal Care and Use Committee of Astellas Pharma Inc. Furthermore, Astellas Pharma Inc., Tsukuba Research Center was awarded Accreditation Status by the Association for Assessment and Accreditation of Laboratory Animal Care International (AAALAC International).

### <Results>

### 1. Aptamer Selection (SELEX) and Characterization

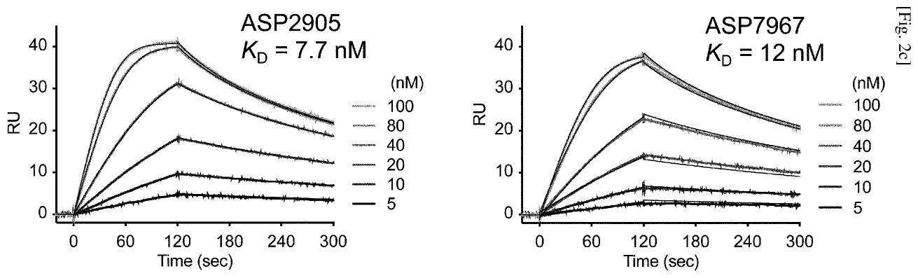

Conventional SELEX requires immobilization of the target molecule on solid phase on which an RNA pool containing randomized sequences are applied. We synthesized a carboxylate derivative of ASP7967 (3) which was coupled to agarose beads containing amino groups (EAH Sepharose 4B). Starting with a random RNA pool containing 40-nucleotide degenerate bases with approximately 6×10¹⁵ unique sequences³⁷, we executed ten rounds of affinity selection with increasing stringency (Table 2). The sequences enriched during the SELEX experiment were analyzed by high-throughput (Illumina) sequencing. After extensive screening of the individual sequences that were enriched after SELEX (data not shown), we identified the sixth most abundant sequence in the final pool (R10-6) as a promising lead aptamer with a putative binding motif that includes the randomized region (FIG. 2b). The minimal binding motif AC17-4 (FIG. 2b) was confirmed to bind both ASP2905 and ASP7967 with comparable affinity by surface plasmon resonance (SPR) (FIG. 2c). AC17-4 bound ASP2905 and ASP7967 with a K_{D} of 7.7 nM and 12 nM for ASP2905 and ASP7967, respectively, at 25° C. Binding of AC17-4 and ASP2905 at 37° C. was also measured by isothermal titration calorimetry (ITC) yielding a K_{D} of 48 nM (FIG. 3).

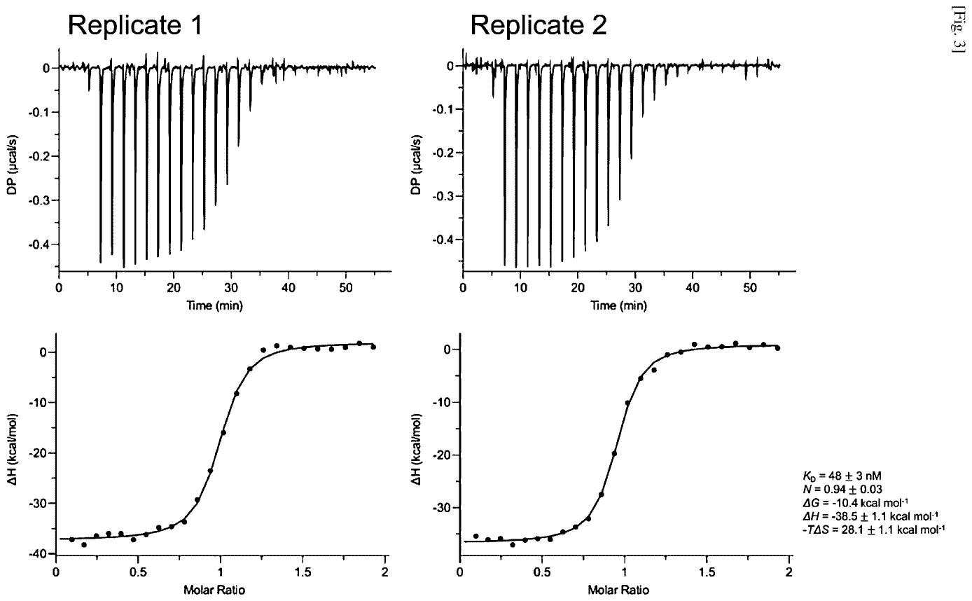

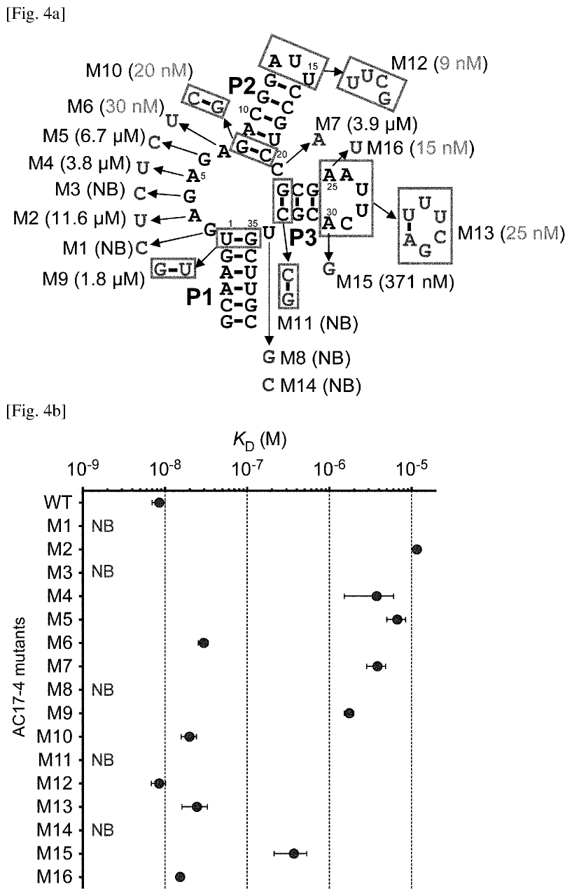

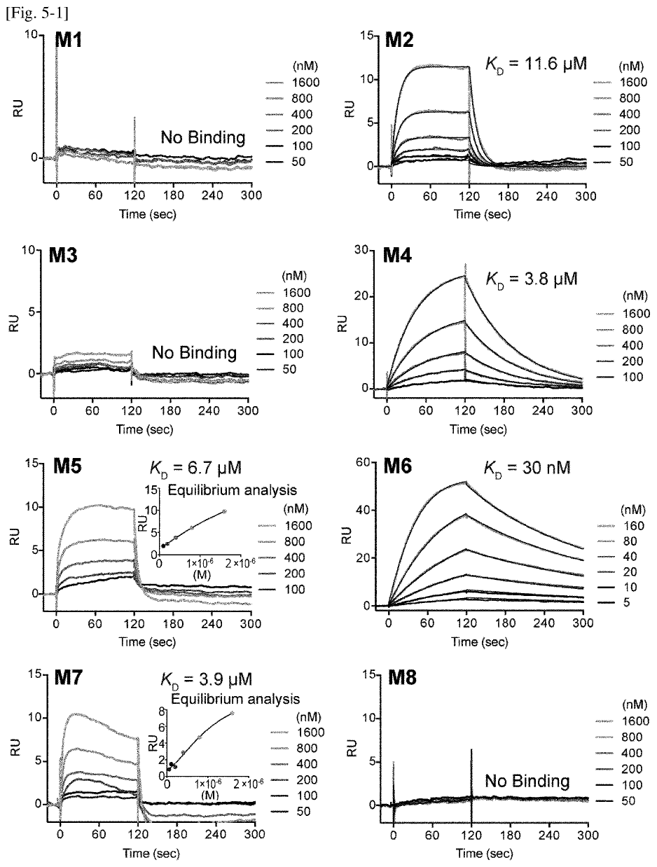

Mutational analysis of the AC17-4 aptamer was performed using SPR (FIG. 4 and FIG. 5). The affinities of the mutants generally confirm the secondary structure depicted in FIG. 4a. Single mutations in G2C, A3U, G4C, A5U, G6C, C20A, A30G, U34C, and U34G abolished or significantly compromised binding, suggesting that these nucleotides play critical roles in aptamer structure or binding. The loops L₂ and L₄ could be substituted with a canonical UUCG tetraloop (M12, M13) without loss of affinity, therefore, are not likely to be involved in aptamer-ligand or tertiary interactions. Putative base-pair substitutions M9 and M11 negatively affected binding, which raise some questions about these interactions. Further biochemical and structural investigations of the aptamer-ligand interaction are ongoing to improve affinity and riboswitch design strategy.

### 2. Mammalian Riboswitches

Next, we sought to regulate gene expression in mammalian cells using AC17-4 and the small molecule ligands. One of the most widely adopted strategies for regulating gene expression in mammalian cells based on aptamer-ligand interaction is to insert one or more allosteric self-cleaving ribozymes (aptazymes) in the untranslated regions (UTRs) of the mRNA encoding the gene of interest^{5,6}. Ribozyme self-cleavage (activated or inhibited by the aptamer-ligand interaction) results in mRNA degradation and repression of protein expression³⁹.

We recently developed the circularly-permuted pistol (CPP) ribozyme as a scaffold to engineer aptazymes and riboswitches that function in mammalian cells²⁷. CPP was designed by linking the native 5′ and 3′ termini of the natural pistol ribozyme structure 40, and by creating the new termini by breaking the native L₃ loop (FIG. 6a,b). The base sequence of the pistol ribozyme (FIG. 6a) is as follows: 5′-CGUCGUCUGGGCGACGGUAAAUAGGUGUUAGGCCCAGAGCGGCAGGG UUCUUCCCUGCGUCACA-3′ (SEQ ID NO: 66). We showed that this scaffold can accommodate an RNA aptamer at multiple positions to engineer riboswitches that function in mammalian cells. Here, we inserted the AC17-4 aptamer in the linker between P2 and P1 stems along with an anti-ribozyme (anti-Rz) sequence that is complementary to the ribozyme sequence downstream of the aptamer (FIG. 6b). The rationale of the aptazyme design is that the ribozyme is active in the absence of the ligand (gene expression OFF), but aptamer-ligand binding stabilizes an alternative structure in which the anti-Rz forms the base-stem (Papt) of the aptamer. This ligand-bound structure disrupts the ribozyme folding thereby inhibiting self-cleavage (gene expression ON) (FIG. 6b). We have shown that the performance of the switch can be tuned by adjusting the size of anti-Rz.

AC17-4 was inserted in the CPP scaffold as shown in FIG. 6b. The anti-Rz sequence was varied from 6 to 10 nucleotides, and the aptazyme was inserted in the 3′ UTR of the EGFP transcript. The base sequences as shown in FIG. 6b are as follows:

(SEQ ID NO: 67)
5′-CUCUAGACCCUGCGUCACAGCAGCAUGAGAGAGACGGAUUCC
GUCCGCGAAUUCACGCUGCGUCGUCUGGGCGACGGUAAAUAGGUG
UUAGGCCCAGAGCGGCAGGGUACAACU-3′
(SEQ ID NO: 68)
5′-CUCUAGACCCUGCGUCACAGCAGCAGUGAGAGAGACGGAUUC
CGUCCGCGAAUUCACGCUGCGUCGUCUGGGCGACGGUAAAUAGGU
GUUAGGCCCAGAGCGGCAGGGUACAACU-3′
(SEQ ID NO: 69)
5′-CUCUAGACCCUGCGUCACAGCAGCAGAUGAGAGAGACGGAUU
CCGUCCGCGAAUUCACGCUGCGUCGUCUGGGCGACGGUAAAUAGG
UGUUAGGCCCAGAGCGGCAGGGUACAACU-3′
(SEQ ID NO: 70)
5′-CUCUAGACCCUGCGUCACAGCAGCAGACUGAGAGAGACGGAU
UCCGUCCGCGAAUUCACGCUGCGUCGUCUGGGCGACGGUAAAUAG
GUGUUAGGCCCAGAGCGGCAGGGUACAACU-3′
(SEQ ID NO: 71)
5′-CUCUAGACCCUGCGUCACAGCAGCAGACCUGAGAGAGACGGA
UUCCGUCCGCGAAUUCACGCUGCGUCGUCUGGGCGACGGUAAAUA
GGUGUUAGGCCCAGAGCGGCAGGGUACAACU-3′

Here, in each base sequence, EGFP sequence is linked to the 5′ end, and a poly-A tail is linked to the 3′ end.

The EGFP-riboswitch plasmid and an mCherry-expressing plasmid (transfection control) were cotransfected into HEK293 cells in the absence or presence (5 μM) of ASP2905 or ASP7967. EGFP and mCherry fluorescence was measured two days after transfection, and EGFP fluorescence was normalized by mCherry fluorescence to account for variability in transfection efficiency. As expected, weak anti-Rz resulted in low EGFP levels and strong anti-Rz resulted in elevated EGFP levels regardless of the presence or absence of the ligand. The optimum switch response was observed with 8-nucleotide anti-Rz (a8-AC17-4-CPP) which activated EGFP expression by approximately 10-fold in the presence of ASP2905 or ASP7967 (FIG. 6c). A notable deviation from the trend is the 7-nucleotide anti-Rz (a7-AC17-4-CPP) that showed higher ON and OFF expression levels. We attribute this to the coincidental complementarity of the nucleotides preceding anti-Rz (CA) to the ribozyme sequence, making the effective anti-Rz sequence length longer than intended. Nonetheless, semi-rational tuning of the anti-Rz length again proved to be an effective strategy for optimization of the CPP aptazymes.

The riboswitch a8-AC17-4-CPP was further analyzed for the dose dependent response to ASP7967 (FIG. 6d). The riboswitch response was mostly saturated at 5 μM ASP7967 with an EC₅₀ of approximately 1.1 μM. The fully induced ON level of a8-AC17-4-CPP was approximately 50% of that of the empty vector. As seen in FIG. 6c and our previous work²⁷, the ON and OFF levels as well as the ON/OFF ratio can be fine-tuned to some extent through engineering the aptazyme sequence design.

Proliferation of HEK293 cells was not significantly affected by ASP2905 or ASP7967 up to 10 μM added to the culture medium (FIG. 7). Consequently, we proceeded to explore the possibility of using the riboswitches to chemically regulate gene expression in mice.

3. Regulation of hEPO Expression in Mice

To demonstrate riboswitch function in vivo, we sought to regulate hEPO expression in mice using an adeno-associated virus (AAV) vector. hEPO is commonly used for the treatment of anemia associated with chronic kidney disease⁴¹, and it represents a class of genes that may benefit from chemically-regulated expression from a gene therapy vector. After a preliminary screening of additional riboswitch variants (data not shown), we decided to use a8c-AC17-4-CPP for the in vivo study due to the low basal expression (OFF) level of the riboswitch variant in HEK293 cells (FIG. 8). The base sequences as shown in FIG. 8a are as follows: 5′-CUCUAGACCCUGCGUCA-CAAAGAAAAGACGACGUGAGAGAGACGGAUUCCGUCCGCGAAUUCACGC UGCGUCGUCUGGGCGACGGUAAAUAGGUGUUAGGCCCAGAGCGGCAGGG UACAACU-3′ (SEQ ID NO: 72) Here, EGFP sequence is linked to the 5′ end, and a poly-A tail is linked to the 3′ end.

Then, a8c-AC17-4-CPP was inserted into the 3′ UTR region of hEPO gene expressed from the CMV promoter in a plasmid containing AAV2 inverted terminal repeats (ITR) (pAAV-CMV-hEPO-a8c-AC17-4-CPP) (FIG. 9a). The plasmid pAAV-CMV-hEPO-a8c-AC17-4-CPP was transfected into HEK293 cells in the absence or presence (0.1~10 μM) of ASP7967. Two days after transfection, hEPO concentrations in the culture medium were measured by ELISA. In the absence of the ligand, hEPO secretion was attenuated to 2.8% compared to the control plasmid that lacks the riboswitch (pAAV-CMV-hEPO-control). As expected, in the presence of 10 μM of the ligand, the hEPO level in the medium reached 29% relative to that of the control, corresponding to a 10-fold induction by ASP7967 (FIG. 9b). These results confirm that the riboswitch functions in the context of the AAV vector to regulate hEPO expression.

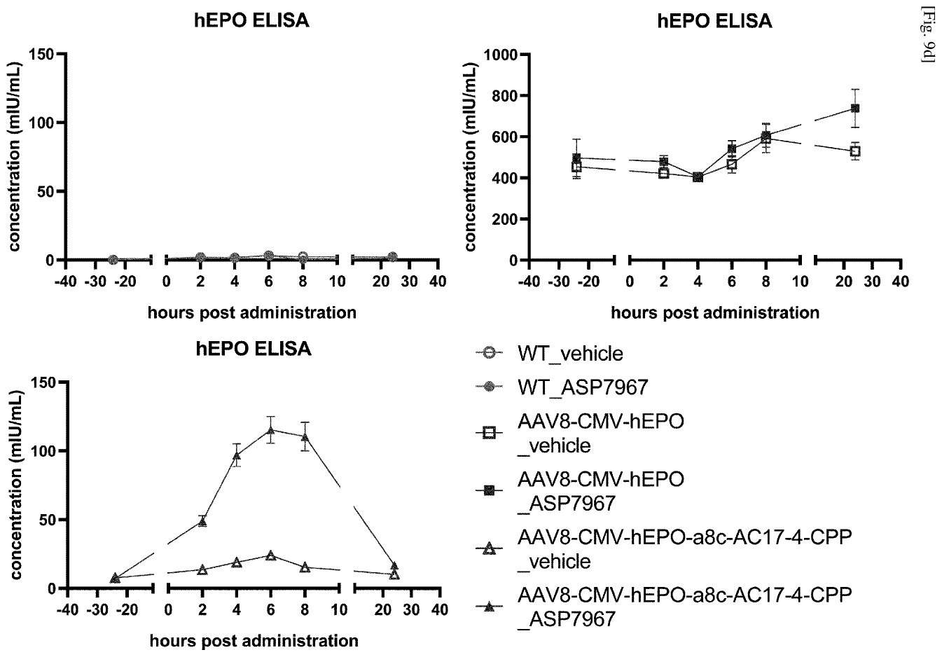

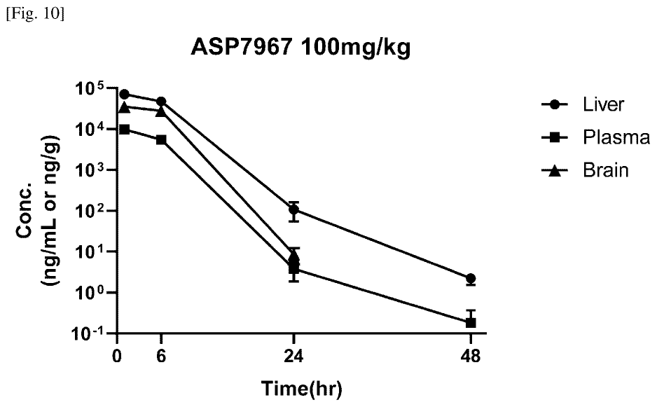

Next, we studied the riboswitch potency and functionality in vivo. Because of the high concentration of ASP7967 in the liver after oral administration (FIG. 10), we selected the liver as a target tissue and selected AAV8 due to its strong liver tropism. Mice were intravenously injected with viral vectors AAV8-CMV-hEPO-control, AAV8-CMV-hEPO-a8c-AC17-4-CPP, or saline (WT). Two weeks after AAV injection, 100 mg kg⁻¹ dose of ASP7967 was orally administered to induce expression from the riboswitch-regulated vector. Blood was drawn before and at multiple time points after the oral administration (FIG. 9c). Secreted hEPO levels in the serum of the mice injected with the control AAV (AAV8-CMV-hEPO-control) were around 550 mIU ml⁻¹, and administration of 100 mg kg⁻¹ dose of the ligand had no effect during the observed time period (FIG. 9d, upper right). On the other hand, upon administration of 100 mg kg⁻¹ dose of the ligand, hEPO secretion levels were induced by riboswitch-regulated vector (AAV8-CMV-hEPO-a8c-AC17-4-CPP) reaching approximately 115 mIU mL-1 at 6-8 h postdosing, corresponding to a 7.2-fold increase over the mice dosed with the vehicle (FIG. 9d, lower left). The serum hEPO level subsequently decreased in accordance with the corresponding decrease of the ligand concentration in the liver (FIG. 10).

Moderate ON/OFF ratio of ~10 exhibited by our riboswitches is typical of mammalian riboswitches^{5,6}. Tighter regulation of gene expression is desirable for many applications. A recently disclosed patent⁴² describes a riboswitch mechanism based on exon skipping induced by aptamer-ligand interaction. In this strategy, a suicide exon containing an internal stop codon flanked by two introns is inserted within the gene whose expression is regulated (FIG. 11a). An aptamer is placed immediately downstream of the 5′-splice site (5′-ss) of the second intron. In the absence of the ligand, the suicide exon is included in the spliced transcript resulting in an OFF state. Aptamer-ligand interaction induces a stable stem masking the 5′-ss which causes exon skipping, resulting in expression of the desired protein (FIG. 11a). The key variable is the stability of the base aptamer stem (P1); if it is too unstable, the suicide exon is constitutively incorporated, while the exon is always skipped if the P1 stem is too stable.

We inserted the exon-skipping switch module between the 169th and 170th codons of EGFP. Varying the size of the P1 stem revealed that an 8-bp stem (ex169-AC17-4-a8) resulted in robust activation of EGFP expression with an ON/OFF ratio of 114 (FIG. 11b). To search for riboswitches with higher ON levels, we screened variants with different P1 sequences/stabilities (data not shown). One of the variants ex169-AC17-4-a9+g2g7 displayed ~75% EGFP expression in the presence of ASP7967 relative to the control with a lower ON/OFF ratio of 58. To further improve its ON/OFF ratio, CPP-based aptazyme variants CPP-AC17-4-a9-P3-9d and CPP-AC17-4-a9-P3-9e (FIG. 12) were inserted into the 3′ UTR. The base sequences as shown in FIG. 12 are as follows:

(SEQ ID NO: 73)
5′-CUCUAGACCCUGCGUCACAUGAGAGAGACGGAUUCCGUCCGC
GAAUUCACGCUGUGUGACACGUCGUCUGGGCGACGGUAAAUAGGU
GUUAGGCCCAGAGCGGCAAGGUCUAACU-3′
(SEQ ID NO: 74)
5′-CUCUAGACCCUGCGUCACAUGAGAGAGACGGAUUCCGUCCGC
GAAUUCACGCUGUGUGACACGUCGUCUGGGCGACGGUAAAUAGGU
GUUAGGCCCAGAGCGGCAGAGUCUAACU-3′

Here, in each base sequence, EGFP sequence is linked to the 5′ end, and a poly-A tail is linked to the 3′ end.

The dual exon-skipping/aptazyme constructs a9+g2g7/CPP-4a9-P3-9d and a9+g2g7/CPP-4a9-P3-9e showed excellent ON/OFF ratios of 177 and 296 with the ON levels of 64% and 52% relative to the control, respectively.

### Discussion

Although synthetic riboswitches promise a number of advantages for mammalian applications such as low risk of immunogenic complications and small genetic size of the constructs⁶, there are some remaining challenges for broader adoption by researchers. Here, we sought to expand the limited repertoire of aptamer-ligand pairs that can be used to construct mammalian riboswitches. ASP2905 was originally developed as an inhibitor of potassium voltage-gated channel sub-family H member 3 (KCNH3) to study its effects on cognitive performance of animals³⁵. The small molecule has been used in vitro in CHO cells stably expressing KCNH3 and cultured rat hippocampal neurons. Oral administration ASP2905 up to 10 mg kg⁻¹ in rats has been reported³⁴. Although no results have been disclosed, ASP2905 has undergone a phase I clinical trial targeting Alzheimer's disease and schizophrenia (Annual Report 2010, Astellas, Inc., https://www.astellas.com/system/files/annual2010_en_0.pdf). ASP2905 is commercially available from several sources.

We performed SELEX against ASP7967 which contains an extra fluorine in the pendant phenyl group of ASP2905 due to the better synthetic accessibility of 3 which was immobilized on agarose beads. Extensive screening of the aptamer candidates enriched after ten rounds of SELEX led us to AC17-4 with a compact core motif of 35 nt (FIG. 2b). AC17-4 bound ASP2905 and ASP7967 with similar affinity (FIG. 2c). Although the mode of binding of these compounds to the aptamer is not clear at this moment, mutational analysis (FIG. 4b) and structural studies may allow further improvement of the aptamer-ligand interaction. However, a notable advantage of RNA-based genetic devices such as riboswitches is that detailed structural information is not always necessary to engineer such devices. Here, we hypothesized that the putative P1 stem of AC17-4 (FIG. 4a) would be stabilized upon binding the ligand as have been observed with other aptamers and inserted it into the CPP ribozyme scaffold which we previously used to construct guanine- and tetracycline-responsive riboswitches²⁷.

We discovered a8-AC17-4-CPP that functions as an ON-switch in response to ASP2905 and ASP7967. Gene expression in cultured mammalian cells was upregulated by ~10-fold in the presence of 5 μM ligand (FIG. 6c). Notably, most other aptamer ligands used for mammalian riboswitches require concentrations 100 μM or higher in the culture medium to fully activate or repress gene expression^{16,20,33}. The observed ON/OFF ratios are also among the best for aptazyme-based mammalian ON-switches reported to date^{12,16,18,20,21,23,26,27,30,43}. However, the moderate baseline expression (OFF level) of these riboswitches often precludes application that require tighter gene regulation. A recent patent disclosed by Meira GTX⁴² claims outstanding ON/OFF ratios of riboswitches based on an exon-skipping mechanism (FIG. 11a). We adapted the riboswitch architecture to our AC17-4 aptamer and observed excellent switching characteristics, in particular, low baseline expression levels (FIG. 11b,c). Combination of an exon-skipping switch and a CPP aptazyme further improved the ON/OFF ratio to 296. This level of switch performance should significantly expand potential applications.

We have demonstrated that our riboswitch can regulate transgene expression in mice in response to oral administration of ASP7967. Oral administration of 100 mg kg⁻¹ ASP7967 upregulated the serum hEPO concentration by ~7.2-fold over the vehicle control (FIG. 9d). Since transgene expression levels in vivo depend on the pharmacokinetics of the ligand in the target tissue, the pharmacokinetics of ASP7967 was evaluated to verify the relationship between the target tissue concentration and the efficacy. It was found that the transgene expression diminished as the ligand concentration in the target tissue declined (FIG. 9d, FIG. 10). Moreover, readministration of the ligand resulted in reactivation of hEPO (data not shown). Our riboswitch can stimulate transgene expression when the ligand is administered as needed, and the transgene expression ceases as the ligand is cleared from the plasma and target tissues. Therefore, riboswitch-regulated gene therapy vectors may be able to reduce the side effects caused by overexpression of transgenes such as MeCP^{244,45}, insulin^{46,47}, and erythropoietin⁴⁸ by appropriate administration of the ligand.

In addition to the riboswitches based on small molecule-aptamer binding, several chemically regulated riboswitches in mammalian cells and in animal models have been recently reported. Monteys et al. used LMI070, a small molecule drug in a clinical trial for treatment of spinal muscular atrophy (SMA), as a trigger to induce inclusion of a synthetic exon49. The molecule works by stabilizing U1 small nuclear RNA interactions near the splice site. While the engineered switch was optimized to function at low LMI070 concentrations where most endogenous splicing events are not affected, prolonged exposure to the compound may result in adverse effects. The riboswitch modules are also somewhat large, requiring 1.16 kbp or 560 bp for the full size and the minimized modules, respectively. It may also be more challenging to develop orthogonal switches that respond to additional molecules based on this strategy. Moreover, as opposed to aptamers that have been harnessed to regulate gene expression through multiple distinct mechanisms⁶, the use of LMI070 will likely be restricted to systems based on regulation of pre-mRNA splicing. Alternatively, morpholino oligonucleotides have been used to interfere with ribozyme cleavage to achieve high ON/OFF ratios⁵⁰. One major challenge, however, is delivery of the oligonucleotide effectors for in vivo applications⁵¹.

Lack of small molecules and their aptamers that function efficiently in vivo or in clinical settings remains a major challenge for biomedical applications of synthetic riboswitches⁵². However, development of new small molecule-aptamer pairs for applications in mammalian cells and animals has been rare. This work represents such an effort resulting in new mammalian riboswitches with improved characteristics. Further improvement of the riboswitch performance (sensitivity to the inducer molecule, ON/OFF ratio, etc.) may be possible by optimization of the aptamer sequence and/or chemical modification of ASP2905/ASP7967.

<Circularly-Permuted AC17-4 (cpAC17-4)>

Circularly-permuted AC17-4 (cpAC17-4) was designed based on AC17-4 aptamer (SEQ ID NO: 50). The base sequence of cpAC17-4 is shown below.

(SEQ ID NO: 77)
5′-GGUGUCCGCGAAUUCACGCUGCUUGUUCGCAAGUGAGAGAGA
CACC-3′

Affinity measurement of the cpAC17-4 aptamer with the ligands by SPR was performed as described above. For this measurement, cpAC17-4 RNA aptamer containing 3′ poly(A) tail was synthesized and used.

(SEQ ID NO: 82)
5′-GGUGUCCGCGAAUUCACGCUGCUUGUUCGCAAGUGAGAGAGA
CACCAAAAAAAAAAAAAAAAAAAAAAAA-3′

The affinity measurement determined that cpAC17-4 bounds ASP2905 with a K_{D} of 30 nM.

Exon-skipping riboswitch cassettes containing a cpAC17-4 aptamer (ex169-cpAC17-4-a7, ex169-cpAC17-4-a8, ex169-cpAC17-4-a9) were prepared by replacing the AC17-4 aptamer with it (core sequence; SEQ ID NO: 83) in the exon-skipping riboswitch cassettes (ex169-AC17-4-a7, ex169-AC17-4-a8, 169-AC17-4-a9; Table 6). The exon-skipping riboswitch cassette was inserted between the 169th and 170th codons in the EGFP coding sequence in pEGFP-BsaI-Amp.

(SEQ ID NO: 83)
5′-CCGCGAATTCACGCTGCTTGTTCGCAAGTGAGAGAG-3′

TABLE 7
Table 7. Riboswitch sequences (exon-skipping)

| Riboswitch | Sequence (5′-3′)¹ |
|---|---|
| ex169-cpAC17-4- | 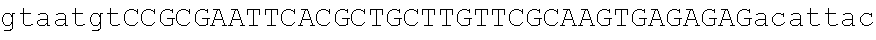 |
| a7 | (SEQ ID NO: 84) |
| ex169-cpAC17-4- | 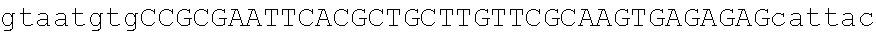 |
| a8 | (SEQ ID NO: 85) |
| ex169-cpAC17-4- | 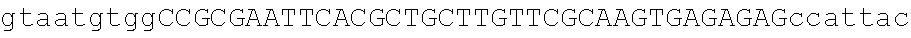 |
| a9 | (SEQ ID NO: 86) |

¹Sequences are shown in DNA. cpAC17-4 aptamer is highlighted in shading.

Riboswitch assay for the exon-skipping riboswitch with the cassette containing cpAC17-4 aptamer was performed in the same manner as for ex169-AC17-4-a7 etc. as described above. The assay confirmed that cpAC17-4 functions as a part of a riboswitch in HEK293 cells like AC17-4.

### REFERENCES

1. Buskirk, A. R. & Liu, D. R. Creating small-molecule-dependent switches to modulate biological functions. Chem. Biol. 12, 151-61 (2005).
2. Xie, M. & Fussenegger, M. Designing cell function: assembly of synthetic gene circuits for cell biology applications. Nat. Rev. Mol. Cell Biol. 19, 507-525 (2018).
3. Gossen, M. & Bujard, H. Tight control of gene expression in mammalian cells by tetracycline-responsive promoters. Proc. Natl. Acad. Sci. U.S.A 89, 5547-51 (1992).
4. Gossen, M. et al. Transcriptional activation by tetracyclines in mammalian cells. Science 268, 1766-9 (1995).
5. Auslander, S. & Fussenegger, M. Synthetic RNA-based switches for mammalian gene expression control. Curr. Opin. Biotechnol. 48, 54-60 (2017).
6. Yokobayashi, Y. Aptamer-based and aptazyme-based riboswitches in mammalian cells. Curr. Opin. Chem. Biol. 52, 72-78 (2019).
7. Panchapakesan, S. S. S. & Breaker, R. R. The case of the missing allosteric ribozymes. Nat. Chem. Biol. 17, 375-382 (2021).
8. Garst, A. D., Edwards, A. L. & Batey, R. T. Riboswitches: structures and mechanisms. Cold Spring Harb. Perspect. Biol. 3 (2011).
9. Dunn, M. R., Jimenez, R. M. & Chaput, J. C. Analysis of aptamer discovery and technology. Nat. Rev. Chem. 1, 0076 (2017).
10. Berens, C., Groher, F. & Suess, B. RNA aptamers as genetic control devices: the potential of riboswitches as synthetic elements for regulating gene expression. Biotechnol. J. 10, 246-57 (2015).
11. Auslander, S., Ketzer, P. & Hartig, J. S. A ligand-dependent hammerhead ribozyme switch for controlling mammalian gene expression. Mol. Biosyst. 6, 807-14 (2010).
12. Chen, Y. Y., Jensen, M. C. & Smolke, C. D. Genetic control of mammalian T-cell proliferation with synthetic RNA regulatory systems. Proc. Natl. Acad. Sci. U.S.A 107, 8531-6 (2010).
13. Ketzer, P., Haas, S. F., Engelhardt, S., Hartig, J. S. & Nettelbeck, D. M. Synthetic riboswitches for external regulation of genes transferred by replication-deficient and oncolytic adenoviruses. Nucleic Acids Res. 40, e167 (2012).
14. Nomura, Y., Zhou, L., Miu, A. & Yokobayashi, Y. Controlling mammalian gene expression by allosteric hepatitis delta virus ribozymes. ACS Synth. Biol. 2, 684-9 (2013).
15. Bell, C. L. et al. Control of alphavirus-based gene expression using engineered riboswitches. Virology 483, 302-11 (2015).
16. Xiang, J. S. et al. Massively parallel RNA device engineering in mammalian cells with RNA-Seq. Nat. Commun. 10, 4327 (2019).
17. Pu, Q. et al. Intracellular Selection of Theophylline-Sensitive Hammerhead Aptazyme. Mol. Ther. Nucleic Acids 20, 400-408 (2020).
18. Beilstein, K., Wittmann, A., Grez, M. & Suess, B. Conditional control of mammalian gene expression by tetracycline-dependent hammerhead ribozymes. ACS Synth. Biol. 4, 526-34 (2015).
19. Zhong, G., Wang, H., Bailey, C. C., Gao, G. & Farzan, M. Rational design of aptazyme riboswitches for efficient control of gene expression in mammalian cells. Elife 5 (2016).
20. Strobel, B. et al. High-throughput identification of synthetic riboswitches by barcode-free amplicon-sequencing in human cells. Nat. Commun. 11, 714 (2020).
21. Strobel, B. et al. A Small-Molecule-Responsive Riboswitch Enables Conditional Induction of Viral Vector-Mediated Gene Expression in Mice. ACS Synth. Biol. 9, 1292-1305 (2020).
22. Lin, Y. H. & Chang, K. Y. Rational design of a synthetic mammalian riboswitch as a ligand-responsive-1 ribosomal frame-shifting stimulator. Nucleic Acids Res. 44, 9005-9015 (2016).
23. Dhamodharan, V., Nomura, Y., Dwidar, M. & Yokobayashi, Y. Optochemical control of gene expression by photocaged guanine and riboswitches. Chem. Commun. (Camb.) 54, 6181-6183 (2018).
24. Kim, N. & Yokobayashi, Y. Novel RNA Viral Vectors for Chemically Regulated Gene Expression in Embryonic Stem Cells. ACS Synth. Biol. 10, 2959-2967 (2021).
25. Kobori, S., Nomura, Y., Miu, A. & Yokobayashi, Y. High-throughput assay and engineering of self-cleaving ribozymes by sequencing. Nucleic Acids Res. 43, e85 (2015).
26. Mustafina, K., Fukunaga, K. & Yokobayashi, Y. Design of Mammalian ON-Riboswitches Based on Tandemly Fused Aptamer and Ribozyme. ACS Synth. Biol. 9, 19-25 (2020).
27. Mustafina, K., Nomura, Y., Rotrattanadumrong, R. & Yokobayashi, Y.

Circularly-Permuted Pistol Ribozyme: A Synthetic Ribozyme Scaffold for Mammalian Riboswitches. ACS Synth. Biol. 10, 2040-2048 (2021).
28. Nomura, Y., Kumar, D. & Yokobayashi, Y. Synthetic mammalian riboswitches based on guanine aptazyme. Chem. Commun. (Camb.) 48, 7215-7 (2012).
29. Sporing, M., Boneberg, R. & Hartig, J. S. Aptamer-Mediated Control of Polyadenylation for Gene Expression Regulation in Mammalian Cells. ACS Synth. Biol. 9, 3008-3018 (2020).
30. Stifel, J., Sporing, M. & Hartig, J. S. Expanding the toolbox of synthetic riboswitches with guanine-dependent aptazymes. Synth. Biol. (Oxf.) 4, ysy022 (2019).
31. Strobel, B. et al. Riboswitch-mediated Attenuation of Transgene Cytotoxicity Increases Adeno-associated Virus Vector Yields in HEK-293 Cells. Mol. Ther. 23, 1582-91 (2015).
32. Takahashi, K. & Yokobayashi, Y. Reversible Gene Regulation in Mammalian Cells Using Riboswitch-Engineered Vesicular Stomatitis Virus Vector. ACS Synth. Biol. 8, 1976-1982 (2019).
33. Groher, F. et al. Riboswitching with ciprofloxacin-development and characterization of a novel RNA regulator. Nucleic Acids Res. 46, 2121-2132 (2018).
34. Takahashi, S., Ohmiya, M., Honda, S. & Ni, K. The KCNH3 inhibitor ASP2905 shows potential in the treatment of attention deficit/hyperactivity disorder. PLOS One 13, e0207750 (2018).
35. Takahashi, S. et al. Neurochemical and neuropharmacological characterization of ASP2905, a novel potent selective inhibitor of the potassium channel KCNH3. Eur. J. Pharmacol. 810, 26-35 (2017).
36. Takahashi, S., Okamura, A., Yamazaki, M. & Ni, K. ASP2905, a specific inhibitor of the potassium channel Kv12.2 encoded by the Kenh3 gene, is psychoactive in mice. Behav. Brain Res. 378, 112315 (2020).
37. Dwidar, M. et al. Programmable Artificial Cells Using Histamine-Responsive Synthetic Riboswitch. J. Am. Chem. Soc. 141, 11103-11114 (2019).
38. Zuker, M. Mfold web server for nucleic acid folding and hybridization prediction. Nucleic Acids Res. 31, 3406-15 (2003).
39. Yen, L. et al. Exogenous control of mammalian gene expression through modulation of RNA self-cleavage. Nature 431, 471-6 (2004).
40. Weinberg, Z. et al. New classes of self-cleaving ribozymes revealed by comparative genomics analysis. Nat. Chem. Biol. 11, 606-10 (2015).
41. Hayat, A., Haria, D. & Salifu, M. O. Erythropoietin stimulating agents in the management of anemia of chronic kidney disease. Patient Prefer Adherence 2, 195-200 (2008).
42. Boyne, A. R., Danos, F. O., Volles, J. M. & Guo, X. Regulation of gene expression by aptamer-mediated modulation of alternative splicing. WO/2016/126747 (2016).
43. Reid, C. A., Nettesheim, E. R., Connor, T. B. & Lipinski, D. M. Development of an inducible anti-VEGF rAAV gene therapy strategy for the treatment of wet AMD. Sci. Rep. 8, 11763 (2018).
44. Collins, A. L. et al. Mild overexpression of MeCP2 causes a progressive neurological disorder in mice. Hum. Mol. Genet. 13, 2679-89 (2004).
45. Gadalla, K. K. E. et al. Development of a Novel AAV Gene Therapy Cassette with Improved Safety Features and Efficacy in a Mouse Model of Rett Syndrome. Mol. Ther. Methods Clin. Dev. 5, 180-190 (2017).
46. Gan, S. U. et al. Correction of Murine Diabetic Hyperglycemia With A Single Systemic Administration of An AAV2/8 Vector Containing A Novel Codon Optimized Human Insulin Gene. Curr. Gene Ther. 16, 65-72 (2016).
47. Thule, P. M. et al. Long-term glycemic control with hepatic insulin gene therapy in streptozotocin-diabetic mice. J. Gene Med. 17, 141-52 (2015).
48. Zhang, M. et al. Erythropoietin promotes abdominal aortic aneurysms in mice through angiogenesis and inflammatory infiltration. Sci. Transl. Med. 13, eaaz4959 (2021).
49. Monteys, A. M. et al. Regulated control of gene therapies by drug-induced splicing. Nature 596, 291-295 (2021).
50. Zhong, G. et al. A reversible RNA on-switch that controls gene expression of AAV-delivered therapeutics in vivo. Nat. Biotechnol. 38, 169-175 (2020).
51. Roberts, T. C., Langer, R. & Wood, M. J. A. Advances in oligonucleotide drug delivery. Nat. Rev. Drug Discov. 19, 673-694 (2020).
52. Tickner, Z. J. & Farzan, M. Riboswitches for Controlled Expression of Therapeutic Transgenes Delivered by Adeno-Associated Viral Vectors. Pharmaceuticals (Basel) 14, 554 (2021).
53. Chang, A. L., McKeague, M., Liang, J. C. & Smolke, C. D. Kinetic and equilibrium binding characterization of aptamers to small molecules using a label-free, sensitive, and scalable platform. Anal. Chem. 86, 3273-8 (2014).
54. Kibbe, W. A. OligoCalc: an online oligonucleotide properties calculator. Nucleic Acids Res. 35, W43-6 (2007).
55. Salado, I. G. et al. Optimization of the pharmacokinetic properties of potent antitrypanosomal triazine derivatives. Eur. J. Med. Chem. 151, 18-26 (2018).
56. Dwidar, M. et al. Programmable Artificial Cells Using Histamine-Responsive Synthetic Riboswitch. J. Am. Chem. Soc. 141, 11103-11114 (2019).

1. An RNA aptamer that binds to ASP7967 or an analogue thereof, the aptamer comprising a sequence: 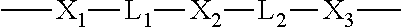
wherein
X₁ has the sequence Y₁GY₂GY₃Y₄Y₅,
L₁ is a first stem loop nucleotide sequence comprising a first stem region, a first loop region and a second stem region, wherein the first stem region and the second stem region are 2 or more base pairs long and are substantially complementary to each other;
X₂ is A, G, C or U,
L₂ is a second stem loop nucleotide sequence comprising a third stem region, a second loop region and a fourth stem region, wherein the third stem region and the fourth stem region are 2 or more base pairs long and are substantially complementary to each other; and wherein the first base in the third stem region is G and the last base in the fourth stem region is C;
X₃ has the sequence UY₆; and
Y₁, Y₂, Y₃, Y₄, Y₅, and Y₆ are, each independently, A, G, C or U; or
a sequence: 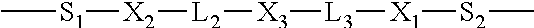
wherein
S₁ and S₂ are, each independently, A, G, C or U, and S₁ and S₂ are capable of forming a base pair or a wobble base pair with each other;
L₃ is a third stem loop nucleotide sequence comprising a fifth stem region, a third loop region and a sixth stem region, wherein the fifth stem region and the sixth stem region are 1 or more base pairs long and are substantially complementary to each other; and
X₁, X₂, X₃, and L₂ are as defined above; or
a sequence: 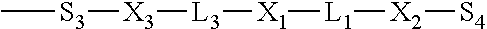
wherein
S₃ is C and S₄ is G; and
X₁, X₂, X₃, L₁, and L₃ are as defined above.

2. The RNA aptamer according to claim 1,
wherein
Y₂ is selected from A or U;
Y₃ is selected from A or U; and/or
Y₄ is selected from G or C.

3. (canceled)

4. The RNA aptamer according to claim 1,
wherein
Y₁ and Y₆ are capable of forming a base pair or a wobble base pair with each other.

5. (canceled)

6. The RNA aptamer according to claim 1,
wherein
the first stem region and the second stem region are 3 to 7 base pair long and are substantially complementary to each other
the first loop region has 3 to 7 bases;
the third stem region and the fourth stem region have 1 to 5 base pairs and are substantially complementary to each other;
the second loop region has 3 to 7 bases;
the fifth stem region and the sixth stem region are 3 to 7 base pairs long and are substantially complementary to each other; and/or
the third loop region has 3 to 7 bases.

7. (canceled)

8. The RNA aptamer according to claim 1,
wherein
the first stem region has the sequence GACGG and the second stem region has the sequence CCGUC.

9. (canceled)

10. The RNA aptamer according to claim 1,
wherein
the first loop region has the sequence AUU or UUCG.

11. (canceled)

12. (canceled)

13. The RNA aptamer according to claim 1,
wherein
the third stem region has the sequence GCG and the fourth stem region has the sequence CGC; or
the third stem region has the sequence GCGU and the fourth stem region has the sequence ACGC.

14. (canceled)

15. The RNA aptamer according to claim 1,
wherein
the second loop region has the sequence AAUUCA or UUCG.

16. The RNA aptamer according to claim 1, wherein the RNA aptamer comprises the sequence: -X₁-L₁-X₂-L₂-X₃-, and further comprises a fifth stem region adjacent to the 5′ terminus of X₁ and a sixth stem region adjacent to the 3′ terminus of X₃, wherein the fifth stem region and the sixth stem region have 1 to 15 base pairs and are substantially complementary to each other, and wherein the fifth stem region and the sixth stem region form a double-stranded stem.

17. (canceled)

18. (canceled)

19. The RNA aptamer according to claim 1,
wherein
the fifth stem region has the sequence CUUG and the sixth stem region has the sequence CAAG.

20. (canceled)

21. The RNA aptamer according to claim 1,
wherein
the third loop region has the sequence UUCG.

22. The RNA aptamer according to claim 1,
wherein
S₁ is C and S₂ is G; or
S₁ is G and S₂ is C.

23. The RNA aptamer according to claim 1, wherein the RNA aptamer comprises the sequence: -S₁-X₂-L₂-X₃-L₃-X₁-S₂-, and further comprises a seventh stem region adjacent to the 5′ terminus of S₁ and a eighth stem region adjacent to the 3′ terminus of S₂, wherein the seventh stem region and the eighth stem region have 1 to 15 base pairs and are substantially complementary to each other, and wherein the seventh stem region and the eighth stem region form a double-stranded stem.

24. The RNA aptamer according to claim 1, wherein the RNA aptamer comprises the sequence: -S₃-X₃-L₃-X₁-L₁-X₂-S₄—, and further comprises a ninth stem region adjacent to the 5′ terminus of S₅ and a tenth stem region adjacent to the 3′ terminus of S₄, wherein the ninth stem region and the tenth stem region have 1 to 15 base pairs and are substantially complementary to each other, and wherein the ninth stem region and the tenth stem region form a double-stranded stem.

25. The RNA aptamer according to claim 1,
wherein the RNA aptamer is circularly permuted.

26. (canceled)

27. A polynucleotide comprising the RNA aptamer according to claim 1, a DNA sequence that is capable of being transcribed into the RNA aptamer according to claim 1, a riboswitch comprising the RNA aptamer according to claim 1, or a DNA sequence that is capable of being transcribed into the riboswitch.

28. (canceled)

29. (canceled)

30. The polynucleotide according to claim 1, further comprising a target sequence, wherein the target sequence encodes a protein; or wherein the target sequence is either an siRNA, pre-miRNA, pri-miRNA, sgRNA, lncRNA, RNA aptamer, ribozyme, tRNA, or rRNA; or a DNA sequence that is capable of being transcribed into an siRNA, pre-miRNA, primiRNA, sgRNA, lncRNA, RNA aptamer, ribozyme, tRNA, or rRNA.

31. An isolated polynucleotide comprising:
a riboswitch comprising an RNA aptamer capable of binding to ASP7967 or an analogue thereof, or a DNA sequence that is capable of being transcribed into the riboswitch and
a target sequence encoding a protein
wherein the riboswitch is operably linked to the target sequence such that expression of the protein is upregulated or downregulated in response to ASP7967 or the analogue thereof.

32. (canceled)

33. (canceled)

34. (canceled)

35. The polynucleotide according to claim 31, wherein
the riboswitch further comprises a self-cleaving ribozyme.

36. The polynucleotide according to claim 35, wherein the self-cleaving ribozyme is activated when the aptamer binds to ASP7967 or an analogue thereof or wherein the self-cleaving ribozyme is inactivated when the aptamer binds to ASP7967 or an analogue thereof.

37. (canceled)

38. (canceled)

39. (canceled)

40. (canceled)

41. A method for regulating expression of a protein in vivo, comprising
introducing the polynucleotide according to claim 31 or a vector comprising the polynucleotide according to claim 31 into a cell, and
bringing ASP7967 or an analogue thereof into contact with the polynucleotide or the vector.

42. A method for treating or preventing a disease, comprising
introducing the polynucleotide according to claim 31 or a vector comprising the polynucleotide according to claim 31 into a subject, and
administering ASP7967 or an analogue thereof into the subject.

43. (canceled)

44. (canceled)

45. (canceled)

46. (canceled)
